(19) United States
(12) Patent Application Publication
(10) Pub. No.: US 2023/0134083 A1
(43) Pub. Date: May 4, 2023

(54) SINGLE-ARM ACTRIIA AND ACTRIIB HETEROMULTIMERS AND METHODS FOR TREATING RENAL DISEASES OR CONDITIONS

(71) Applicant: Acceleron Pharma Inc., Cambridge, MA (US)

(72) Inventors: Gang Li, Cambridge, MA (US); Ravindra Kumar, Cambridge, MA (US)

(21) Appl. No.: 17/911,347

(22) PCT Filed: Mar. 11, 2021
(86) PCT No.: PCT/US2021/021991

§ 371 Date: Sep. 13, 2022

Related U.S. Application Data
(60) Provisional application No. 62/989,037, filed on Mar. 13, 2020.

(51) Int. Cl. C07K 19/00; A61K 38/13; A61K 38/17; A61K 47/68; C07K 14/71

(52) U.S. Cl. CPC C07K 19/00; A61K 38/13; A61K 38/1774; A61K 38/1796; A61K 47/68; C07K 14/71; C07K 2317/52; C07K 2317/526; C07K 2319/30; C07K 2319/32

(57) ABSTRACT

In some aspects, the disclosure relates to single-arm AetRIIA heteromultimers and sing-arm ActRIIB heteromultimers and methods of using such heteromultimers to treat, prevent, or reduce tire progression rate and/or severity of renal diseases or conditions, particularly treating, preventing or reducing the progression rate and/or severity of one or more renal-associated complications. The disclosure also provides methods of using a single-arm ActRIIA heteromultimer or single-arm ActRIIB heteromultimer to treat, prevent, or reduce the progression rate and/or severity of a variety of conditions including, but not limited to, Alport syndrome, focal segmental glomerulosclerosis (FSGS), polycystic kidney disease, and/or chronic kidney disease.

### CROSS-REFERENCE TO RELATED APPLICATION

This application claims the benefit of and priority to U.S. Provisional Application No. 62/989,037, filed Mar. 13, 2020. The foregoing application is incorporated herein by reference in its entirety.

### BACKGROUND OF THE INVENTION

Renal diseases include a range of conditions that can lead to loss of kidney function. and, in some cases, can be fatal. Normally-functioning kidneys filter wastes and excess fluids from the blood, which are then excreted in urine. For example, when chronic kidney disease reaches an advanced stage, dangerous levels of fluid, electrolytes and wastes can build up in the bloodstream. If left untreated, renal disease can progress to end-stage renal disease (e.g., end-stage kidney failure), which is fatal without artificial filtering (dialysis) or a kidney transplant. Thus, there is a high, unmet need for effective therapies for treating renal diseases or conditions (e.g., Alport syndrome, focal segmental glomerulosclerosis (FSGS), polycystic kidney disease, chronic kidney disease).

### SUMMARY OF THE INVENTION

In part, the disclosure provides single-arm heteromultimeric complexes comprising a single ActRIIA or a single ActRIIB polypeptide, including fragments and variants thereof. These constructs may be referred to herein as single-arm heteromultimers, a single-arm ActRIIA heteromultimer or heterodimer, and single-arm ActRIIB heteromultimer or heterodimer. Optionally, single-arm polypeptide heteromultimers disclosed herein (e.g., a single-arm ActRIIB heteromultimer, such as a single-arm ActRIIB heterodimer Fc fusion) have different ligand-binding specificities/profiles compared to a corresponding heteromultimer (e.g., an ActRIIB homodimer Fc fusion). Novel properties are exhibited by heteromultimers comprising a single domain of an ActRIIA or a single domain of an ActRIIB polypeptide, as shown by Examples herein.

Heteromultimeric structures include, for example, heterodimers, heterotrimers, and higher order complexes. Preferably, ActRIIA or ActRIIB polypeptides as described herein comprise a ligand-binding domain of the receptor, for example, an extracellular domain of an ActRIIA or ActRIIB receptor. Accordingly, in certain aspects, heteromultimers described herein comprise an extracellular domain of an ActRIIA or ActRIIB polypeptide, as well as truncations and variants thereof. Preferably, ActRIIA or ActRIIB polypeptides as described herein, as well as heteromultimers comprising the same, are soluble. In certain aspects, heteromultimers of the disclosure bind to one or more ActRIIA or ActRIIB ligands (e.g., activin A, activin B, GDF11, GDF8, GDF3, BMP5, BMP6, and BMP10. Optionally, protein complexes of the disclosure bind to one or more of these ligands with a K_{D} of less than or equal to 10⁻⁸, 10⁻⁹, 10⁻¹⁰, 10⁻¹¹, or 10⁻¹². In general, single-arm heteromultimers of the disclosure antagonize (inhibit) one or more activities of at least one ActRIIA or ActRIIB ligand, and such alterations in activity may be measured using various assays known in the art, including, for example, a cell-based assay as described herein. Preferably, single-arm heteromultimers of the disclosure exhibit a serum half-life of at least 4, 6, 12, 24, 36, 48, or 72 hours in a mammal (e.g., a mouse or a human). Optionally, single-arm heteromultimers of the disclosure may exhibit a serum half-life of at least 6, 8, 10, 12, 14, 20, 25, or 30 days in a mammal (e.g., a mouse or a human).

In part, the disclosure provides ActRIIA or ActRIIB single-arm heteromultimers that can be used to treat renal diseases or conditions (e.g., Alport syndrome, focal segmental glomerulosclerosis (FSGS), polycystic kidney disease, chronic kidney disease). Positive effects were observed for a single-arm ActRIIB heteromultimer in the UUO and Col4a3 (−/−) Alport syndrome models. The disclosure establishes that antagonists of the ActRII (e.g., ActRIIA and ActRIIB) signaling pathways may be used to reduce the severity of a renal disease or condition (e.g., Alport syndrome, focal segmental glomerulosclerosis (FSGS), polycystic kidney disease, chronic kidney disease), and that desirable therapeutic agents may be selected on the basis of ActRII signaling antagonist activity. Therefore, in some embodiments, the disclosure provides methods for using various single-arm ActRIIA heteromultimers or single-arm ActRIIB heteromultimers for treating renal diseases or conditions, including but not limited to Alport syndrome, focal segmental glomerulosclerosis (FSGS), polycystic kidney disease, and chronic kidney disease, including, for example, single-arm heteromultimers that inhibit one or more ActRIIA or ActRIIB ligands [e.g., activin A, activin B, GDF11, GDF8, GDF3, BMP6, BMP5, and BMP10].

In some embodiments, the present disclosure provides methods of treating renal diseases or conditions, comprising administering a single-arm ActRIIB heteromultimer to a subject in need thereof. In some embodiments, the present disclosure provides methods of treating renal diseases or conditions comprising administering a single-arm ActRIIB heteromultimer to a subject in need thereof, the heteromultimer comprising a first polypeptide covalently or non-covalently associated with a second polypeptide, wherein the first polypeptide comprises an amino acid sequence of a first member of an interaction pair and an amino acid sequence of ActRIIB; and the second polypeptide comprises an amino acid sequence of a second member of the interaction pair, and wherein the second polypeptide does not comprise ActRIIB.

In some embodiments, the ActRIIB polypeptide comprises, consists, or consists essentially of an amino acid sequence that is: at least 70%, 80%, 85%, 90%, 91%, 92%, 93%, 94% 95%, 96%, 97%, 98%, 99% or 100% identical to the sequence of any of SEQ ID Nos: 1, 2, 3, 4, 5, and 6; or at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119,120, 121, 122, 123, 124, 125, 126, 127, 128, 129, 130, 131, 132, 133, or 134 of SEQ ID NO: 1.

In some embodiments, an ActRIIB polypeptide may comprise an amino acid sequence that is at least 70%, 75%, 80%, 85%, 90%, 91%, 92%, 93%, 94%, 95%, 96%, 97%, 98%, 99%, or 100% identical to the amino acid sequence of SEQ ID NO: 79. In some embodiments, an ActRIIB polypeptide may comprise an amino acid sequence that is at least 70%, 75%, 80%, 85%, 90%, 91%, 92%, 93%, 94%, 95%, 96%, 97%, 98%, 99%, or 100% identical to the amino acid sequence of SEQ ID NO: 79, wherein the ActRIIB polypeptide comprises an acidic amino acid at position 79 with respect to SEQ ID NO: 1. In certain embodiments, ActRIIB polypeptides to be used in accordance with the methods and uses described herein do not comprise an acidic amino acid at the position corresponding to L79 of SEQ ID NO: 1. In certain embodiments, ActRIIB polypeptides to be used in accordance with the methods and uses described herein do not comprise an aspartic acid (D) at the position corresponding to L79 of SEQ ID NO: 1.

In some embodiments, the present disclosure provides methods of treating renal diseases or conditions, comprising administering a single-arm ActRIIA heteromultimer to a subject in need thereof. In some embodiments, the present disclosure provides methods of treating renal diseases or conditions comprising administering a single-arm ActRIIA heteromultimer to a subject in need thereof, the heteromultimer comprising a first polypeptide covalently or non-covalently associated with a second polypeptide, wherein the first polypeptide comprises an amino acid sequence of a first member of an interaction pair and an amino acid sequence of ActRIIA; and the second polypeptide comprises an amino acid sequence of a second member of the interaction pair, and wherein the second polypeptide does not comprise ActRIIA.

In some embodiments, the ActRIIA polypeptide comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID Nos: 9, 10, and 11; or at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments of the present disclosure, the heteromultimer is a heterodimer.

In some embodiments of the present disclosure, the first member of an interaction pair comprises a first constant region from an IgG heavy chain. In some embodiments, the first constant region from an IgG heavy chain is a first immunoglobulin Fc domain. In some embodiments, the first constant region from an IgG heavy chain comprises an amino acid sequence that is at least 70%, 80%, 85%, 900, 91%, 92%, 93%, 94% 95%, 96%, 97%, 98%, 99% or 100% identical to a sequence selected from any one of SEQ ID NOs: 14-28.

In some embodiments of the present disclosure, the second member of an interaction pair comprises a second constant region from an IgG heavy chain. In some embodiments, the second constant region from an IgG heavy chain is a first immunoglobulin Fc domain. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments of the present disclosure, the first polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 46, 48, 55, 57, 58, 59, 60, 61, 84, 86, 88, 89, 90, and 91. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 49, 51, 62, 63, 85, and 87.

In some embodiments of the present disclosure, a single-arm ActRIIB heteromultimer comprises a linker domain positioned between the ActRIIB polypeptide and the first member of an interaction pair. In some embodiments. the linker domain comprises an amino acid sequence selected from any one of SEQ ID NOs: 29-44.

In some embodiments of the present disclosure, a single-arm ActRIIA heteromultimer comprises a linker domain positioned between the ActRIIA polypeptide and the first member of an interaction pair. In some embodiments, the linker domain comprises an amino acid sequence selected from any one of SEQ ID NOs: 29-44.

In some embodiments of the present disclosure, the first polypeptide and/or second polypeptide comprises one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, the first polypeptide and/or second polypeptide is glycosylated and has a glycosylation pattern obtainable from expression of the first polypeptide and/or second polypeptide in a CHO cell.

In some embodiments, the heteromultimer (e.g., heterodimer Fc fusion) binds to one or more of ActRIIA or ActRIIB ligands selected from the group consisting of activin A, activin B, GDF11, GDF8, GDF3, BMP5, BMP6, and BMP10. In some embodiments, a single-arm ActRIIB heterodimer Fc fusion has greater ligand selectivity than an ActRIIB homodimer Fc fusion. In some embodiments, an ActRIIB homodimer Fc fusion binds strongly to activin A, activin B, GDF11, GDF8, and BMP10. In some embodiments, a single-arm ActRIIB heterodimer Fc fusion binds strongly to activin B and GDF11 and binds intermediately to GDF8 and activin A. In some embodiments, a single-arm ActRIIB heterodimer Fc fusion displays weak, minimal, or undetectable binding to BMP10. In some embodiments, a single-arm ActRIIB heterodimer Fc fusion antagonizes activin A, activin B, GDF8, and GDF11 and minimally antagonizes one or more of BMP9, BMP10, BMP6, and GDF3. In some embodiments, single-arm ActRIIA heterodimer Fc fusion exhibits preferential binding to activin A over activin B combined with greatly enhanced selectivity for activin A over GDF11. In some embodiments, a single-arm ActRIIA heterodimer Fc fusion largely retains intermediate binding to GDF8 and BMP10 as observed with an ActRIIA homodimer Fc fusion. In some embodiments, a single-arm ActRIIA heterodimer Fc fusion is utilized in therapeutic applications where it is desirable to antagonize activin A preferentially over activin B while minimizing antagonism of GDF11. In some embodiments of the present disclosure, a single-arm ActRIIA heterodimer Fc fusion or a single-arm ActRIIB heterodimer Fc fusion inhibits the activity of one or more ActRIIA or ActRIIB ligands in a cell-based assay.

In some embodiments of the present disclosure, the renal disease or condition is Alport syndrome. In some embodiments, the renal disease or condition is focal segmental glomerulosclerosis (FSGS). In some embodiments, the FSGS is primary FSGS. In some embodiments, the FSGS is secondary FSGS. In some embodiments, the FSGS is genetic FSGS. In some embodiments the renal disease or condition is autosomal dominant polycystic kidney disease (ADPKD). In some embodiments, the renal disease or condition is autosomal recessive polycystic kidney disease (ARPKD). In some embodiments, the renal disease or condition is chronic kidney disease (CKD).

In some embodiments, methods of the present disclosure comprise further administering to the subject an additional active agent and/or supportive therapy for treating a renal disease or condition. In some embodiments, the additional active agent and/or supportive therapy for treating a renal disease or condition is selected from the group consisting of: an angiotensin receptor blocker (ARB) (e.g., losartan, irbesartan, olmesartan, candesartan, valsartan, fimasartan, azilsartan, salprisartan, and telmisartan), an angiotensin-converting enzyme (ACE) inhibitor (e.g., benazepril, captopril, enalapril, lisinopril, perindopril, ramipril, trandolapril, and zofenopril), a glucocorticoid (e.g., beclomethasone, betamethasone, budesonide, cortisone, dexamethasone, hydrocortisone, methylprednisolone, prednisolone, methylprednisone, prednisone, and triamcinolone), a calcineurin inhibitor (e.g., cyclosporine, tacrolimus), cyclophosphamide, chlorambucil, a janus kinase inhibitor (e.g., tofacitinib), an mTOR inhibitor (e.g., sirolimus, everolimus), an IMDH inhibitor (e.g., azathioprine, leflunomide, mycophenolate), a biologic (e.g., abatacept, adalimumab, anakinra, basiliximab, certolizumab, daclizumab, etanercept, fresolimumab, golimumab, infliximab, ixekizumab, natalizumab, rituximab, secukinumab, tocilizumab, ustekinumab, vedolizumab), a statin (e.g., benazepril, valsartan, fluvastatin, pravastatin), lademirsen (anti-miRNA-21), bardoxolone methyl. Achtar gel, tolvaptan, abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, VX-147, kidney dialysis, kidney transplant, mesenchymal stem cell therapy, bone marrow stem cells, lipoprotein removal, a Liposorber LA-15 device, plasmapheresis, plasma exchange, and a change in diet (e.g., dietary sodium intake). In some embodiments, an additional active agent and/or supportive therapy for treating a renal disease or condition is an angiotensin receptor blocker (ARB) selected from the group consisting of losartan, irbesartan, olmesartan, candesartan, valsartan, fimasartan, azilsartan, salprisartan, and telmisartan. In some embodiments, an additional active agent and/or supportive therapy for treating a renal disease or condition is an angiotensin-converting enzyme (ACE) inhibitor selected from the group consisting of benazepril, captopril, enalapril, lisinopril, perindopril, ramipril, trandolapril, and zofenopril. In some embodiments, an additional active agent and/or supportive therapy for treating a renal disease or condition is a combination of an ARB and an ACE inhibitor.

In some embodiments. methods of the present disclosure reduce severity, occurrence and/or duration of one or more of albuminuria, proteinuria, microalbuminuria, and macroalbuminuria in a subject in need thereof. In some embodiments of the present disclosure, the subject has proteinuria. In some embodiments, the subject has albuminuria. In some embodiments, the subject has moderate albuminuria. In some embodiments, the subject has severe albuminuria. In some embodiments, the subject has an albumin-creatinine ratio (ACR) of between about 30 and about 300 mg albumin per 24 hours of urine collection. In some embodiments, the subject has an ACR of between about 30 and about 300 mg albumin/g of creatinine. In some embodiments, the subject has an albumin-creatinine ratio (ACR) of above about 300 mg albumin/24 hours. In some embodiments, the subject has an ACR of above about 300 mg albumin/g of creatinine. In some embodiments, the subject has Stage A1 albuminuria. In some embodiments, the subject has Stage A2 albuminuria. In some embodiments, the subject has Stage A3 albuminuria. In some embodiments, the present disclosure provides methods of reducing severity, occurrence and/or duration of Stage A 1 albuminuria. In some embodiments, the present disclosure provides methods of reducing severity, occurrence and/or duration of Stage A2 albuminuria. In some embodiments, the present disclosure provides methods of reducing severity, occurrence and/or duration of Stage A3 albuminuria. In some embodiments, methods of the present disclosure delay or prevent a subject with Stage A1 albuminuria from progressing to Stage A2 albuminuria. In some embodiments, methods of the present disclosure delay or prevent a subject with Stage A2 from progressing to Stage A3 albuminuria. In some embodiments, methods of the present disclosure delay or prevent worsening of albuminuria stage progression in a subject in need thereof. In some embodiments, methods of the present disclosure improve albuminuria classification in a subject by one or more stages.

In some embodiments, methods of the present disclosure reduce an ACR of the subject. In some embodiments, the method reduces the subject's ACR by between about 0.1 and about 100.0 mg albumin/g creatinine (e.g., by between about 0.1 and about 2.5 mg albumin/g, between about 2.5 and about 3.5 mg albumin/g creatinine, between about 3.5 and about 5.0 mg albumin/g creatinine, between about 5.0 and about 7.5 mg albumin/g creatinine, between about 7.5 and about 10.0 mg albumin/g creatinine, between about 10.0 and about 15.0 mg albumin/g creatinine, between about 15.0 and about 20.0 mg albumin/g creatinine, between about 20.0 and about 25.0 mg albumin/g creatinine, between about 30.0 and about 35.0 mg albumin/g creatinine, between about 40.0 and about 45.0 mg albumin/g creatinine, between about 45.0 and about 50.0 mg albumin/g creatinine, between about 50.0 and about 60.0 mg albumin/g creatinine, between about 60.0 and about 70.0 mg albumin/g creatinine, between about 70.0 and about 80.0 mg albumin/g creatinine, between about 80.0 and about 90.0 mg albumin/g creatinine, between about 90.0 and about 100.0 mg albumin/g creatinine). In some embodiments, the method reduces the subject's ACR by at least 2.5% (e.g., 5%, 10%, 15%, 20%, 25%, 30%, 40%, 50%, 60%, 70%, 80%, 90%, 95%, or 99%) compared to a baseline measurement.

In some embodiments, methods of the present disclosure reduce a urinary protein-creatinine ratio (UPCR) of the subject. In some embodiments, the method reduces the subject's UPCR by between about 0.1 and about 100.0 mg urinary protein/mg creatinine (e.g., by between about 0.1 and about 2.5 mg urinary protein/mg creatinine, between about 2.5 and about 3.5 mg urinary protein/mg creatinine, between about 3.5 and about 5.0 mg urinary protein/mg creatinine, between about 5.0 and about 7.5 mg urinary protein/mg creatinine, between about 7.5 and about 10.0 mg urinary protein/mg creatinine, between about 10.0 and about 15.0 mg urinary protein/mg creatinine, between about 15.0 and about 20.0 mg urinary protein/mg creatinine, between about 20.0 and about 25.0 mg urinary protein/mg creatinine, between about 30.0 and about 35.0 mg urinary protein/mg creatinine, between about 40.0 and about 45.0 mg urinary protein/mg creatinine, between about 45.0 and about 50.0 mg urinary protein/mg creatinine, between about 50.0 and about 60.0 mg urinary protein/mg creatinine, between about 60.0 and about 70.0 mg urinary protein/mg creatinine, between about 70.0 and about 80.0 mg urinary protein/mg creatinine, between about 80.0 and about 90.0 mg urinary protein/mg creatinine, between about 90.0 and about 100.0 mg urinary protein/mg creatinine).

In some embodiments, methods of the present disclosure reduce a urinary protein-creatinine ratio (UPCR) of the subject. In some embodiments, the method reduces the subject's UPCR by between about 0.1 and about 100.0 g urinary protein/g creatinine (e.g., by between about 0.1 and about 2.5 g urinary protein/g creatinine, between about 2.5 and about 3.5 g urinary protein/g creatinine, between about 3.5 and about 5.0 g urinary protein/g creatinine, between about 5.0 and about 7.5 g urinary protein/g creatinine, between about 7.5 and about 10.0 g urinary protein/g creatinine, between about 10.0 and about 15.0 g urinary protein/g creatinine, between about 15.0 and about 20.0 g urinary protein/g creatinine, between about 20.0 and about 25.0 g urinary protein/g creatinine, between about 30.0 and about 35.0 g urinary protein/g creatinine, between about 40.0 and about 45.0 g urinary protein/g creatinine, between about 45.0 and about 50.0 g urinary protein/g creatinine, between about 50.0 and about 60.0 g urinary protein/g creatinine, between about 60.0 and about 70.0 g urinary protein/g creatinine, between about 70.0 and about 80.0 g urinary protein/g creatinine, between about 80.0 and about 90.0 g urinary protein/g creatinine, between about 90.0 and about 100.0 g urinary protein/g creatinine). In some embodiments, the method reduces the subject's absolute UPCR by greater than or equal to 0.5 g urinary protein/g creatinine compared to a baseline measurement. In some embodiments, the method reduces the subject's UPCR to less than 0.5 g urinary protein/g creatinine compared to a baseline measurement. In some embodiments, the method reduces the subject's UPCR to less than 0.3 g urinary protein/g creatinine compared to a baseline measurement.

In some embodiments, the method reduces the subject's UPCR by at least 2.5% (e.g., 5%, 10%, 15%, 20%, 25%, 30%, 40%, 50%, 60%, 70%, 80%, 90%, 95%, or 99%) compared to a baseline measurement. In some embodiments, the method reduces the subject's UPCR by greater than or equal to 30% compared to a baseline measurement. In some embodiments. the method reduces the subject's UPCR by greater than or equal to 40% compared to a baseline measurement. In some embodiments, the method reduces the subject's UPCR by greater than or equal to 50% compared to a baseline measurement.

In some embodiments, methods of the present disclosure increase the subject's estimated glomerular filtration rate (eGFR) and/or glomerular filtration rate (GFR). In some embodiments, the eGFR is measured using serum creatinine, age, ethnicity, and gender variables. In some embodiments, the eGFR is measured using one or more of Cockcroft-Gault formula, Modification of Diet in Renal Disease (MDRD) formula, CKD-EPI formula, Mayo quadratic formula, and Schwartz formula. In some embodiments, the eGFR and/or GFR is increased by at least 2.5% (e.g., 5%, 10%, 15%, 20%, 25%, 30%, 40%, 50%, 60%, 70%, 80%, 90%, 95%, or 99%) compared to a baseline measurement. In some embodiments, the eGFR and/or GFR is increased by greater than or equal to 30% compared to a baseline measurement. In some embodiments, the eGFR and/or GFR is increased by greater than or equal to 40% compared to a baseline measurement.

In some embodiments, the eGFR and/or GFR is increased by about 1 mL/min/1.73 m² (e.g., 3, 5, 7, 9, 10, 15, 20, 25, 30, 35, 40, 45, 50, 55, 60, 65, 70, 75, 80, 85, 90, 95, or 100 mL/min/1.73 m²) compared to a baseline measurement. In some embodiments, the eGFR and/or GFR is increased by about 1 mL/min/year (e.g., 2, 3, 5, 7, 9, 10, 15, 20, 25, 30, 35, 40, 45, 50, 55, 60, 65, 70, 75, 80, 85, 90, 95, or 100 mL/min/year) compared to a baseline measurement. In some embodiments, the eGFR and/or GFR is increased by greater than or equal to 1 mL/min/year compared to a baseline measurement. In some embodiments, the eGFR and/or GFR is increased by greater than or equal to 3 mL/min/year compared to a baseline measurement.

In some embodiments of the present disclosure, the renal disease or condition of a subject is evaluated in stages of chronic kidney disease (CKD). In some embodiments, the subject has stage one chronic kidney disease (CKD). In some embodiments, the subject has stage two chronic kidney disease (CKD). In some embodiments, the subject has stage three chronic kidney disease (CKD). In some embodiments, the subject has stage four chronic kidney disease (CKD). In some embodiments, the subject has stage five chronic kidney disease (CKD). In some embodiments, methods of the present disclosure reduce severity. occurrence and/or duration of Stage 1 CKD. In some embodiments, methods of the present disclosure reduce severity, occurrence and/or duration of Stage 2 CKD. In some embodiments, methods of the present disclosure reduce severity, occurrence and/or duration of Stage 3 CKD. In some embodiments, methods of the present disclosure reduce severity, occurrence and/or duration of Stage 3a CKD. In some embodiments, methods of the present disclosure reduce severity, occurrence and/or duration of Stage 3b CKD. In some embodiments, methods of the present disclosure reduce severity, occurrence and/or duration of Stage 4 CKD. In some embodiments, methods of the present disclosure reduce severity. occurrence and/or duration of Stage 5 CKD. In some embodiments, methods of the present disclosure prevent or delay a subject with Stage 1 CKD from progressing to Stage 2 CKD. In some embodiments, methods of the present disclosure prevent or delay a subject with Stage 2 CKD from progressing to Stage 3 CKD. In some embodiments, methods of the present disclosure prevent or delay a subject with Stage 2 CKD from progressing to Stage 3a CKD. In some embodiments, methods of the present disclosure prevent or delay a subject with Stage 3a CKD from progressing to Stage 3b CKD. In some embodiments, methods of the present disclosure prevent or delay a subject with Stage 3 CKD from progressing to Stage 4 CKD. In some embodiments, methods of the present disclosure prevent or delay a subject with Stage 3b CKD rom progressing to Stage 4 CKD. In some embodiments, methods of the present disclosure prevent or delay a subject with Stage 4 CKD rom progressing to Stage 5 CKD. In some embodiments, methods of the present disclosure prevent or delay worsening of CKD stage progression in a subject in need thereof. In some embodiments, methods of the present disclosure improve renal damage CKD classification in a subject by one or more stages.

In some embodiments, methods of the present disclosure reduce total kidney volume in a subject. In some embodiments, the total kidney volume is reduced by at least 2.5% (e.g., 5%, 10%, 15%, 20%, 25%, 30%, 40%, 50%, 60%, 70%, 80%, 90%, 95/a, or 99%) compared to a baseline measurement.

In some embodiments, methods of the present disclosure reduce the subject's blood urea nitrogen (BUN). In some embodiments, the BUN is reduced by at least 2.5% (e.g., 5%, 10%, 15%, 20%, 25%, 30%, 40%, 50%, 60%, 70%, 80%, 90%, 95%, or 99%) compared to a baseline measurement.

In some embodiments, methods of the present disclosure reduce urine Neutrophil Gelatinase-Associated Lipocalin (uNGAL) concentration in a subject. In some embodiments, the uNGAL is reduced by at least 2.5% (e.g., 5%, 10%, 15%, 20%, 25%, 30%, 40%, 50%, 60%, 70%, 80%, 90%, 95%, or 99%) compared to a baseline measurement. In some embodiments, the subject has a uNGAL measurement of <50 ng/mL, is an indication of low risk of acute kidney injury. In some embodiments, the subject has a uNGAL measurement of between about 50 and about 149 ng/mL, an indication of equivocal risk of acute kidney injury. In some embodiments, the subject has a uNGAL measurement of between about 150 and about 300 ng/mL, an indication of moderate risk of acute kidney injury. In some embodiments, the subject has a uNGAL measurement of >300 ng/mL, an indication of high risk of acute kidney injury. In some embodiments of the present disclosure, the method reduces the subject's uNGAL by between about 0.1 and about 300.0 ng/mL (e.g., by between about 0.1 and about 50 ng/mL, by between about 0.1 and about 100.0 ng/mL, by between about 0.1 and about 150.0 ng/mL, by between about 0.1 and about 200.0 ng/mL, by between about 0.1 and about 250.0 ng/mL, by between about 0.1 and about 300.0 ng/mL, by between about 0.1 and about 25 ng/mL, by between about 25 and about 50 ng/mL, by between about 50 and about 100 ng/mL, by between about 100 and about 150 ng/mL, by between about 150 and about 200 ng/mL, by between about 200 and about 250 ng/mL, by between about 250 and about 300 ng/mL, by more than 300 ng/mL).

In some embodiments, methods of the present disclosure prevent or delay clinical worsening of a renal disease or condition (e.g., Alport syndrome, focal segmental glomerulosclerosis (FSGS), polycystic kidney disease, chronic kidney disease). In some embodiments, methods of the present disclosure reduce risk of hospitalization for one or more complications associated with a renal disease or condition (e.g., Alport syndrome, focal segmental glomerulosclerosis (FSGS), polycystic kidney disease, chronic kidney disease).

In some embodiments, the single-arm ActRIIB heteromultimer or single-arm ActRIIA heteromultimer of the present disclosure is administered subcutaneously. In some embodiments, the single-arm ActRIIB heteromultimer or single-arm ActRIIA heteromultimer is administered once every two weeks. In some embodiments, the single-arm ActRIIB heteromultimer or single-arm ActRIIA heteromultimer is administered once every three weeks. In some embodiments, the single-arm ActRIIB heteromultimer or single-arm ActRIIA heteromultimer is administered once every four weeks.

### BRIEF DESCRIPTION OF THE DRAWINGS

FIG. 1 shows an alignment of extracellular domains of human ActRIIA (SEQ ID NO: 66) and human ActRIIB (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB precursor proteins without their intracellular domains (SEQ ID NOs: 67, 68, 69, 70, 71, 72, respectively) human ActRIIA precursor protein without its intracellular domain (SEQ ID NO: 73), and a consensus ActRII precursor protein (SEQ ID NO: 74).

FIG. 3 shows a multiple sequence alignment of various vertebrate ActRIIA proteins and human ActRIIA (SEQ ID NOs: 75-82).

FIG. 4 shows multiple sequence alignment of Fc domains from human IgG isotypes using Clustal 2.1. Hinge regions are indicated by dotted underline. Double underline indicates examples of positions engineered in IgG1 Fc (SEQ ID NO: 22) to promote asymmetric chain pairing and the corresponding positions with respect to other isotypes IgG2 (SEQ ID NO: 23), IgG3 (SEQ ID NO: 24) and IgG4 (SEQ ID NO: 26).

FIG. 5 shows ligand binding data for a single-arm ActRIIB heterodimer Fc fusion compared to an ActRIIB homodimer Fc fusion. For each protein heteromultimer, ligands are ranked by off-rate (k_{off} or k_{d}), a kinetic constant that correlates well with ligand signaling inhibition, and listed in descending order of binding affinity (ligands bound most tightly are listed at the top). At left, yellow, red, green, and blue lines indicate magnitude of the off-rate constant. Ligands of particular interest are highlighted in bold while others are represented in gray, and solid black lines indicate ligands whose binding to heterodimer is enhanced or unchanged compared with homodimer, whereas dashed lines indicate substantially reduced binding compared with homodimer. As shown, ActRIIB homodimer Fc fusion binds to each of five high affinity ligands with similarly high affinity, whereas single-arm ActRIIB heterodimer Fc fusion discriminates more readily among these ligands. Thus, single-arm ActRIIB heterodimer Fc fusion binds strongly to activin B and GDF11 and with intermediate strength to GDF8 and activin A. In further contrast to ActRIIB homodimer Fc fusion, single-arm ActRIIB heterodimer Fc fusion displays only weak binding to BMP10 and no binding to BMP9. These data indicate that single-arm ActRIIB heterodimer Fc fusion has greater ligand selectivity than homodimeric ActRIIB Fc fusion.

FIG. 6 shows ligand binding data for a single-arm ActRIIA heterodimer Fc fusion compared to ActRIIA homodimer Fc fusion. Format is the same as for FIG. 5. As shown, ActRIIA homodimer Fc fusion exhibits preferential binding to activin B combined with strong binding to activin A and GDF11, whereas single-arm ActRIIA heterodimer Fc fusion has a reversed preference for activin A over activin B combined with greatly enhanced selectivity for activin A over GDF11 (weak binder). These data indicate that single-arm ActRIIA heterodimer Fc fusion has substantially different ligand selectivity than homodimeric ActRIIA Fc fusion.

FIG. 7 shows therapeutic effect of single-arm ActRIIB heterodimer Fc fusion (“sa-IIB-hd”) in a UUO model. Sixteen mice underwent left unilateral ureteral ligation twice at the level of the lower pole of kidney, and after 3 days, they were randomized into two groups: i) “UUO/PBS” (eight mice were injected subcutaneously with vehicle control, phosphate buffered saline (PBS), at days 3, 7, 10, and 14 after surgery) and ii) “UUO/sa-IIB-hd” (eight mice were injected subcutaneously with single-arm ActRIIB heterodimer Fc fusion at a dose of 10 mg/kg at days 3, 7, 10, and 14 after surgery). The “Control” is the contralateral kidney that did not undergo the unilateral ureteral obstruction procedure. FIGS. 7A-7F show gene expression analysis of fibrotic gene markers (Fibronectin, PAI-I, CTGF, Col-I, Col-III, a-SMA, respectively), FIGS. 7G-7H show gene expression analysis of inflammatory gene markers (MCP-1, TNFa, respectively), FIG. 71 shows gene expression analysis of Thrombospondin 1 (Thbs1), FIG. 7J shows gene expression analysis of a kidney injury marker (NGAL), and Figures K-N show gene expression analysis of TGFβ ligands (Tgfb1, Tgfb2, Tgfb3, Activin A, respectively). Relative to “UUO/PBS” treated mice, “UUO/sa-IIB-hd” treated mice demonstrated significantly lower expression of fibrotic and inflammatory genes, reduced upregulation of TGFβ 1/2/3, activin A, and Thbs1, and reduced kidney injury gene expression. Statistical significance (p value) is depicted as * p<0.05, **p<0.01, ***p<0.001, and ****p<0.0001 for comparison between “Control” and sample “UUO/PBS”. Statistical significance (p value) is depicted as #p<0.05, ##p<0.01, ###p<0.001, and ####p<0.0001 for comparison between “Control” and sample “UUO/sa-IIB-hd”. Statistical significance (p value) is depicted as @ p<0.05, @@p<0.01, @@@>p<0.001, and @@@@p<0.0001 for comparison between sample “UUO/PBS” and sample “UUO/sa-IIB-hd”. “B.D.L.” means that the measurement value was below the limit of detection, and no statistics were calculated for a value in comparison to a “B.D.L.” value.

FIG. 8 shows therapeutic effect of single-arm ActRIIB heterodimer Fc fusion protein (“sa-IIB-hd”) in a Col4a3 (−/−) Alport syndrome model. Thirteen Col4a3−/− mice were randomized into two groups: i) “Col4a3 Vehicle” (seven mice injected subcutaneously with vehicle control, phosphate buffered saline (PBS), twice a week) and ii) “Col4a3 sa-IIB-hd (30 mpk)” (six mice injected subcutaneously with single-arm ActRIIB heterodimer Fc fusion at a dose of 30 mg/kg twice a week. Six “WT” mice, which are non-treated Col4a3+/+ mice, were also analyzed at 7.5 weeks. Relative to Col4a3 Vehicle mice, treatment of mice with single-arm ActRIIB heterodimer Fc fusion protein (Col4a3 sa-IIB-hd mice) significantly reduced albuminuria (depicted as an albumin-creatinine ratio (ACR)) by about 49.9% (p<0.0l) (FIG. 8A), which was associated with decreased blood urea nitrogen (BUN) (FIG. 8B) in Col4a3 sa-IIB-hd mice. Statistical significance (p value) is depicted as * p<0.05, **p<0.01, ***p<0.001, and ****p<0.0001.

FIG. 9 shows therapeutic effect of single-arm ActRIIB heterodimer Fc fusion protein (“sa-IIB-hd”) in a Col4a3 (−/−) Alport syndrome model. Fifty-eight Col4a3−/− mice 6 weeks of age were treated with ramipril (ACEi, 10 mg/kg/day) in drinking water and randomized into three groups i) “Col4a3 Vehicle” (twenty-seven mice injected subcutaneously with vehicle control, phosphate buffered saline (PBS), twice a week); ii) “Col4a3 sa-IIB-hd (10 mpk)” (eleven mice injected subcutaneously with single-arm ActRIIB heterodimer Fc fusion protein at a dose of 10 mg/kg twice a week; and iii) “Col4a3 sa-IIB-hd (30 mpk)” (twenty mice injected subcutaneously with single-arm ActRIIB heterodimer Fc fusion protein at a dose of 30 mg/kg twice a week. “WT” mice are Col4a3+/+ mice with no treatment. Relative to Col4a3 Vehicle mice, treatment of mice with single-arm ActRIIB heterodimer Fc fusion protein (Col4a3 sa-IIB-hd mice) both at 10 mg/pk and 30 mg/kg significantly reduced albuminuria in the presence of ACEi (FIG. 9A). In addition, 30 mg/kg treatment significantly decreased urinary NGAL (e.g., uNAGL) levels in the presence of ACEi (FIG. 9B). In the presence of ACEi, “Col4a3 Vehicle” mice had a median survival of 76 days (FIG. 9C). Statistical significance (p value) is depicted as *p<0.05 30 mpk vs Vehicle, ***:p<0.001 30 mpk vs Vehicle, ****p<0.0001 30 mpk vs Vehicle, ##: p<0.01 10 mpk vs Vehicle, and ###p<0.001 10 mpk vs Vehicle.

### DETAILED DESCRIPTION OF THE INVENTION

### 1. Overview

In part, the present disclosure relates to single-arm heteromultimers comprising an extracellular domain of ActRIIA or an extracellular domain of ActRIIB, methods of making such single-arm heteromultimers, and uses thereof. As described herein, single-arm heteromultimers may comprise an extracellular domain of ActRIIA or ActRIIB. In certain preferred embodiments, heteromultimers of the disclosure have an altered profile of binding to ActRIIA or ActRIIB ligands relative to a corresponding homomultimer complex (e.g., an ActRIIB heterodimer Fc fusion compared to an ActRIIB homodimer Fc fusion).

The TGF-β superfamily is comprised of over 30 secreted factors including TGF-betas, activins, nodals, bone morphogenetic proteins (BMPs), growth and differentiation factors (GDFs), and anti-Mullerian hormone (AMH) [Weiss et al (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-beta superfamily (e.g., ligands binding to ActRIIA or ActRIIB) share the same dimeric structure in which the central 3½ turn helix of one monomer packs against the concave surface formed by the beta-strands of the other monomer. The majority of TGF-beta family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGF-beta superfamily (e.g., ActRIIA or ActRIIB) signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massague (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

Activins are members of the TGF-beta superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84: and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a PAB heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the a subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGF-beta superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461: Kambadur et al. Genome Res. (1997) 7:910-915; and Schuelke et al. (2004) N Engl J Med. 350:2682-8]. Studies have also shown that muscle wasting associated with HIV-infection in humans is accompanied by increases in GDF8 protein expression [Gonzalez-Cadavid et al., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263: 6407-6415: Wakefield et al. (1988) J. Biol. Chem., 263; 7646-7654; and Brown et al. (1990) Growth Factors, 3: 35-43]. Other proteins which bind to GDF8 or structurally related proteins and inhibit their biological activity include follistatin, and potentially, follistatin-related proteins [Gamer et al. (1999) Dev. Biol., 208: 222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22: 260-264; and Nakashima et al. (1999) Mech. Dev., 80: 185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, GDF11 may have in vitro and in vivo applications in the treatment of diseases such as muscle diseases and neurodegenerative diseases (e.g., amyotrophic lateral sclerosis).

As used herein ActRII refers to the family of type II activin receptors. This family includes both the activin receptor type IIA (ActRIIA), encoded by the ACVR2A gene, and the activin receptor type JIB (ActRIIB), encoded by the ACVR2B gene. ActRII receptors are TGF-beta superfamily type II receptors that bind a variety of TGF-beta superfamily ligands including activins, GDF8 (myostatin), GDF11, and a subset of BMPs, notably BMP6 and BMP7. ActRII receptors are implicated in a variety of biological disorders including muscle and neuromuscular disorders (e.g., muscular dystrophy, amyotrophic lateral sclerosis (ALS), and muscle atrophy), undesired bone/cartilage growth, adipose tissue disorders (e.g., obesity), metabolic disorders (e.g., type 2 diabetes), and neurodegenerative disorders. See, e.g., Tsuchida et al., (2008) Endocrine Journal 55(1):11-21, Knopf et al., U.S. Pat. No. 8,252,900, and OMIM entries 102581 and 602730.

In certain aspects, the present disclosure relates to the use of single-arm ActRIIA heteromultimers or single-arm ActRIIB heteromultimers comprising an extracellular domain of ActRIIA or ActRIIB, respectively, preferably soluble heteromultimers, to antagonize intracellular signaling transduction (e.g., Smad signaling) initiated by one or more ActRIIA or ActRIIB ligands (e.g., activin A. activin B, GDF11, GDF8, GDF3, BMP5, BMP6 and BMP10). As described herein, single-arm ActRIIA heteromultimers or single-arm ActRIIB heteromultimers may be useful for treating a renal disease or condition (e.g., Alport syndrome, focal segmental glomerulosclerosis (FSGS), polycystic kidney disease, chronic kidney disease).

As demonstrated herein, a single-arm ActRIIB heterodimer Fc fusion is effective in suppressing the expression of fibrotic and inflammatory genes, inhibiting the upregulation of TGFβ 1/2/3, activin A, and Thbs1, and reducing kidney injury. Single-arm ActRIIB heterodimer Fc fusion treatment suppresses kidney fibrosis and inflammation and reduces kidney injury in a UUO model. Furthermore, urinary albumin to creatinine ratio (ACR) was calculated to measure albuminuria, which was significantly increased from 4 weeks to 7.5 weeks in Col4a3−/− mice (“Col4a3 Vehicle”) mice. Treatment of mice with single-arm ActRIIB heterodimer Fc fusion significantly reduced albuminuria by 49.9% (p<0.01), which was associated with decreased BUN in Col4a3−/− mice (“Col4a3 Vehicle”) mice. Regardless of ACE inhibitor treatment, albuminuria was significantly increased from 6 weeks to 10 weeks in Col4a3−/− mice (“Col4a3 Vehicle”). Relative to Col4a3 Vehicle mice, treatment of mice with single-arm ActRIIB heterodimer Fc fusion protein (Col4a3 sa-IIB-hd mice) both at 10 mg/pk and 30 mg/kg significantly reduced albuminuria and increased survival in the presence of ACEi. These data demonstrate that single-arm ActRIIB heterodimer Fc fusion treatment reduces albuminuria and improves renal function in an Alport mouse model. Moreover, these data indicate that other single-arm ActRII heterodimer Fc fusion proteins may be useful in the treatment or preventing of renal diseases or conditions including, for example, single-arm ActRIIA heterodimer Fc fusion protein.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however. % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system. including digital UNIX V4.0D. All sequence comparison parameters are set by the ALIGN-2 program and do not vary.

“Does not substantially bind to X”, in all its grammatical forms, is intended to mean that an agent has a K_{D} that is greater than about 10⁻⁷, 10⁻⁶, 10⁻⁵, 10⁻⁴ or greater (e.g., no detectable binding by the assay used to determine the K_{D}) for “X”.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably <5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification. the word “comprise” or variations such as “comprises” or “comprising” will be understood to imply the inclusion of a stated integer or groups of integers but not the exclusion of any other integer or group of integers.

### 2. Single-Arm Heteromultimers Comprising ActRIIA or ActRIIB Polypeptides

In certain aspects, the disclosure concerns single-arm ActRIIA heteromultimers or single-arm ActRIIB heteromultimers comprising an ActRIIA or ActRIIB polypeptide, respectively. In certain embodiments, the polypeptides disclosed herein may form protein complexes (e.g., heteromultimers) comprising a first polypeptide covalently or non-covalently associated with a second polypeptide, wherein the first polypeptide comprises the amino acid sequence of an ActRIIA or an ActRIIB polypeptide and the amino acid sequence of a first member of an interaction pair; and the second polypeptide comprises the amino acid sequence of a second member of the interaction pair, and wherein the second polypeptide does not comprise an ActRIIA or ActRIIB polypeptide. The interaction pair may be any two polypeptide sequences that interact to form a complex, particularly a heterodimeric complex although operative embodiments may also employ an interaction pair that forms a homodimeric sequence. As described herein, one member of the interaction pair may be fused to an ActRIIA or ActRIIB polypeptide, such as a polypeptide comprising an amino acid sequence that is at least 70%, 75% 80%, 85%, 90%, 91%, 92%, 93%, 94%, 95%, 96%, 97%, 98%, 99% or 100% identical to the sequence of any of SEQ ID NOs: 1, 2, 3, 4, 5, 6, 9, 10, 11, 46, 48, 55, 57, 58, 59, 60, 61, 84, 86, 88, 89, 90, and 91. Preferably, the interaction pair is selected in part to confer an improved serum half-life, or to act as an adapter on to which another moiety, such as a polyethylene glycol moiety, is attached to provide an improved serum half-life relative to the monomeric form of the ActRIIA or ActRIIB polypeptide.

As shown herein, monomeric (single-arm) forms of ActRIIA or ActRIIB can exhibit substantially altered ligand-binding selectivity compared to their corresponding homodimeric forms, but the monomeric forms tend to have a short serum residence time (half-life), which is undesirable in the therapeutic setting. A common mechanism for improving serum half-life is to express a polypeptide as a homodimeric fusion protein with a constant domain portion (e.g., an Fc portion) of an IgG. However, ActRIIA or ActRIIB polypeptides expressed as homodimeric proteins (e.g., in an Fc fusion construct) may not exhibit the same activity profile as the monomeric form. As demonstrated herein, the problem may be solved by fusing the monomeric form to a half-life extending moiety, and surprisingly, this can be readily achieved by expressing such proteins as an asymmetric heterodimeric fusion protein in which one member of an interaction pair is fused to an ActRIIA or ActRIIB polypeptide and another member of the interaction pair is fused to either no moiety or to a heterologous moiety, resulting in a novel ligand-binding profile coupled with an improvement in serum half-life conferred by the interaction pair.

In certain aspects, the present disclosure relates to single-arm heteromultimers comprising an ActRIIA or ActRIIB polypeptide (e.g., a polypeptide comprising the amino acid sequence of any of SEQ ID NOs: 1, 2, 3, 4, 5, 6, 9, 10, 11, 46, 48, 55, 57, 58, 59, 60, 61, 84, 86, 88, 89, 90, and 91), which are generally referred to herein as “single-arm heteromultimers of the disclosure” or “single-arm ActRIIA heteromultimers” or “single-arm ActRIIB heteromultimers”. Preferably, single-arm heteromultimers of the disclosure are soluble, e.g., a single-arm heteromultimer comprises a soluble portion of at least one ActRIIA or ActRIIB polypeptide. In general, the extracellular domains of ActRIIA or ActRIIB correspond to a soluble portion of the ActRIIA or ActRIIB polypeptide. Therefore, in some embodiments, single-arm heteromultimers of the disclosure comprise an extracellular domain of an ActRIIA or ActRIIB polypeptide. Exemplary extracellular domains of ActRIIA and ActRIIB are disclosed herein and such sequences, as well as fragments, functional variants, and modified forms thereof, may be used in accordance with the inventions of the present disclosure (e.g., single-arm heteromultimer compositions and uses thereof). In some embodiments, the amino acid sequence of ActRIIA or ActRIIB extracellular domain may optionally be provided with the C-terminal lysine (K) removed (e.g., SEQ ID NOs: 88 and 89, or 84, 86, and 91 respectively).

A defining structural motif known as a three-finger toxin fold is important for ligand binding by ActRIIA or ActRIIB and is formed by 10, 12, or 14 conserved cysteine residues located at varying positions within the extracellular domain of each monomeric receptor. See, e.g., Greenwald et al. (1999) Nat Struct Biol 6:18-22; Hinck (2012) FEBS Lett 586:1860-1870. Any of the heteromeric complexes described herein may comprise such domain of ActRIIA or ActRIIB. The core ligand-binding domains of ActRIIA or ActRIIB, as demarcated by the outermost of these conserved cysteines, correspond to positions 29-109 of SEQ ID NO: 1 (ActRIIB precursor) and positions 30-110 of SEQ ID NO: 9 (ActRIIA precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 residues on either terminus without necessarily altering ligand binding. Exemplary extracellular domains for N-terminal and/or C-terminal truncation include SEQ ID NOs: 2, 3, 5, 6, 10, 11, and 83.

In other preferred embodiments, single-arm heteromultimers of the disclosure bind to and inhibit (antagonize) activity of one or more ActRIIA or ActRIIB ligands including, but not limited to, activin A, activin B. GDF11, GDF8, GDF3, BMP5, BMP6, and BMP10. In particular, single-arm heteromultimers of the disclosure may be used to antagonize intracellular signaling transduction (e.g., Smad signaling) initiated by one or more TGFβ superfamily ligands (e.g., ActRIIA or ActRIIB ligands). As described herein, such antagonist heteromultimers may be for the treatment or prevention of various TGF-beta associated conditions, including without limitation renal diseases or conditions (e.g., Alport syndrome, focal segmental glomerulosclerosis (FSGS), polycystic kidney disease, chronic kidney disease) that are affected by one or more ligands of the TGF-beta superfamily (e.g., ligands of ActRIIA or ActRIIB). In some embodiments, single-arm heteromultimers of the disclosure have different ligand-binding profiles in comparison to their corresponding homomultimer (e.g., a single-arm ActRIIB heterodimer Fc fusion vs. a corresponding single-arm ActRIIB homodimer Fc fusion). As described herein, single-arm heteromultimers of the disclosure include, e.g., heterodimers, heterotrimers, heterotetramers and further oligomeric structures based on a single-arm unitary complex. In certain preferred embodiments, single-arm heteromultimers of the disclosure are heterodimers.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627 and WO 2008/097541, which are incorporated herein by reference in its entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

(SEQ ID NO: 1)

| | |
|---|---|
| 1 | MTAPWVALAL LWGSLCAGSG RGEAETRECI YYNANWELER |
| | TNQSGLERCE |
| 51 | GEQDKRLHCY ASWRNSSGTI ELVKKGCWLD DFNCYDRQEC |
| | VATEENPQVY |
| 101 | FCCCEGNFCN ERFTHLPEAG GPEVTYEPPP TAPTLLTVLA |
| | YSLLPIGGLS |
| 151 | LIVLLAFWMY RHRKPPYGHV DIHEDPGPPP PSPLVGLKPL |
| | QLLEIKARGR |
| 201 | FGCVWKAQLM NDFVAVKIFP LQDKQSWQSE REIFSTPGMK |
| | HENLLQFIAA |
| 251 | EKRGSNLEVE LWLITAFHDK GSLTDYLKGN IITWNELCHV |
| | AETMSRGLSY |
| 301 | LHEDVPWCRG EGHKPSIAHR DFKSKNVLLK SDLTAVLADF |
| | GLAVRFEPGK |
| 351 | PPGDTHGQVG TRRYMAPEVL EGAINFQRDA FLRIDMYAMG |
| | LVLWELVSRC |
| 401 | KAADGPVDEY MLPFEEEIGQ HPSLEELQEV VVHKKMRPTI |
| | KDHWLKHPGL |
| 451 | AQLCVTIEEC WDHDAEARLS AGCVEERVSL IRRSVNGTTS |
| | DCLVSLVTSV |
| 501 | TNVDLPPKES SI |

The signal peptide is indicated with a single underline: the extracellular domain is indicated in bold font; and the potential, endogenous N-linked glycosylation sites are indicated with a double underline.

A processed extracellular ActRIIB polypeptide sequence is as follows:

(SEQ ID NO: 2)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 3)
GRGEAETRECIYYNANWELERTNQSGLSRCEGEQDKRLK
CYASWRNSSGTIELVKKGCWLDDFNCYDRQECVATEENP
QVYFCCCEGNFCNERFTHLPEA.

A form of ActRIIB with an alanine at position 64 of SEQ ID NO: 1 (Δ64) is also reported in the literature See, e.g., Hilden et al. (1994) Blood, 83(8): 2163-2170. Applicants have ascertained that an ActRIIB-Fc fusion protein comprising an extracellular domain of ActRIIB with the Δ64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore. sequences with an R64 are used as the “wild-type” reference sequence for human ActRIIB in this disclosure.

The form of ActRIIB with an alanine at position 64 is as follows:

(SEQ ID NO: 4)

| | |
|---|---|
| 1 | MTAPWVALAD LWGSLCAGSG RGEAETRECI YYNANWELER |
| | TNQSGLERCE |
| 51 | GEQDKRLHCY ASWANSSGTI ELVKKGCWLD DFNCYDRQEC |
| | VATEENPQVY |
| 101 | FCCCEGNFCN ERFTHLPEAG GPEVTYEPPP TAPTLLTVLA |
| | YSLLPIGGLS |
| 151 | LIVLLAFWMY RHRKPPYGHV DIHEDPGPPP PSPLVGLKPL |
| | QLLEIKARGR |
| 201 | FGCVWKAQLM NDFVAVKIFP LQDKQSWQSE REIFSTPGMK |
| | HENLLQFIAA |
| 251 | EKRGSNLEVE LWLITAFHDK GSLTDYLKGN IITWNELCHV |
| | AETMSRGLSY |
| 301 | LHEDVPWCRG EGHKPSIAHR DFKSKNVLLK SDLTAVLADF |
| | GLAVRFEPGK |
| 351 | PPGDTHGQVG TRRYMAPEVI EGAINFQRDA FLRIDMYAMG |
| | LVLWELVSRC |
| 401 | KAADGPVDEY MLPFEEEIGQ HPSLEELQEV VVHKKMRPTI |
| | KDHWLKHPGL |
| 451 | AQLCVTIEEC WDHDAEARLS AGCVEERVSL IRRSVNGTTS |
| | DCLVSLVTSV |
| 501 | TNVDLPPKES SI |

The signal peptide is indicated by single underline and the extracellular domain is indicated by bold font.

The processed extracellular ActRIIB polypeptide sequence of the alternative Δ64 form is as follows:

(SEQ ID NO: 5)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a Δ15 sequence) is as follows:

(SEQ ID NO: 6)
GRGEAETRECIYYNANWELERTNQSGLERCEGEQDKRLH
CYASWANSSGTIELVKKGCWLDDFNCYDRQECVATEENP
QVYFCCCEGNFCNIRFTHLPEA

A nucleic acid sequence encoding the human ActRIIB precursor protein is shown below (SEQ ID NO: 7), consisting of nucleotides 25-1560 of Genbank Reference Sequence NM_001 106.3. which encode amino acids 1-513 of the ActRIIB precursor. The sequence as shown provides an arginine at position 64 and may be modified to provide an alanine instead. The signal sequence is underlined.

(SEQ ID NO: 7)

| | |
|---|---|
| 1 | ATGACGGCGC CCTGGGTGGC CCTCGCCCTC CTCTGGGGAT |
| | CGCTGTGCGC |
| 51 | CGGCTCTGGG CGTGGGGAGG CTGAGACACG GGAGTGCATC |
| | TACTACAACG |
| 101 | CCAACTGGGA GCTGGAGCGC ACCAACCAGA GCGGCCTGGA |
| | GCGCTGCGAA |
| 151 | GGCGAGCAGG ACAAGCGGCT GCACTGCTAC GCCTCCTGGC |
| | GCAACAGCTC |
| 201 | TGGCACCATC GAGCTCGTGA AGAAGGGCTG CTGGCTAGAT |
| | GACTTCAACT |
| 251 | GCTACGATAG GCAGGAGTGT GTGGCCACTG AGGAGAACCC |
| | CCAGGTGTAC |
| 301 | TTCTGCTGCT GTGAAGGCAA CTTCTGCAAC GAACGCTTCA |
| | CTCATTTGCC |
| 351 | AGAGGCTGGG GGCCCGGAAG TCACGTACGA GCCACCCCCG |
| | ACAGCCCCCA |
| 401 | CCCTGCTCAC GGTGCTGGCC TACTCACTGC TGCCCATCGG |
| | GGGCCTTTCC |
| 451 | CTCATCGTCC TGCTGGCCTT TTGGATGTAC CGGCATCGCA |
| | AGCCCCCCTA |
| 501 | CGGTCATGTG GACATCCATG AGGACCCTGG GCCTCCACCA |
| | CCATCCCCTC |
| 551 | TGGTGGGCCT GAAGCCACTG CAGCTGCTGG AGATCAAGGC |
| | TCGGGGGCGC |
| 601 | TTTGGCTGTG TCTGGAAGGC CCAGCTCATG AATGACTTTG |
| | TAGCTGTCAA |
| 651 | GATCTTCCCA CTCCAGGACA AGCAGTCGTG GCAGAGTGAA |
| | CGGGAGATCT |
| 701 | TCAGCACACC TGGCATGAAG CACGAGAACC TGCTACAGTT |
| | CATTGCTGCC |
| 751 | GAGAAGCGAG GCTCCAACCT CGAAGTAGAG CTGTGGCTCA |
| | TCACGGCCTT |
| 801 | CCATGACAAG GGCTCCCTCA CGGATTACCT CAAGGGGAAC |
| | ATCATCACAT |
| 851 | GGAACGAACT GTGTCATGTA GCAGAGACGA TGTCACGAGG |
| | CCTCTCATAC |
| 901 | CTGCATGAGG ATGTGCCCTG GTGCCGTGGC GAGGGCCACA |
| | AGCCGTCTAT |
| 951 | TGCCCACAGG GACTTTAAAA GTAAGAATGT ATTGCTGAAG |
| | AGCGACCTCA |
| 1001 | CAGCCGTGCT GGCTGACTTT GGCTTGGCTG TTCGATTTGA |
| | GCCAGGGAAA |
| 1051 | CCTCCAGGGG ACACCCACGG ACAGGTAGGC ACGAGACGGT |
| | ACATGGCTCC |
| 1101 | TGAGGTGCTC GAGGGAGCCA TCAACTTCCA GAGAGATGCC |
| | TTCCTGCGCA |
| 1151 | TTGACATGTA TGCCATGGGG TTGGTGCTGT GGGAGCTTGT |
| | GTCTCGCTGC |
| 1201 | AAGGCTGCAG ACGGACCCGT GGATGAGTAC ATGCTGCCCT |
| | TTGAGGAAGA |
| 1251 | GATTGGCCAG CACCCTTCGT TGGAGGAGCT GCAGGAGGTG |
| | GTGGTGCACA |
| 1301 | AGAAGATGAG GCCCACCATT AAAGATCACT GGTTGAAACA |
| | CCCGGGCCTG |
| 1351 | GCCCAGCTTT GTGTGACCAT CGAGGAGTGC TGGGACCATG |
| | ATGCAGAGGC |
| 1401 | TCGCTTGTCC GCGGGCTGTG TGGAGGAGCG GGTGTCCCTG |
| | ATTCGGAGGT |
| 1451 | CGGTCAACGG CACTACCTCG GACTGTCTCG TTTCCCTGGT |
| | GACCTCTGTC |
| 1501 | ACCAATGTGG ACCTGCCCCC TAAAGAGTCA AGCAC |

A nucleic acid sequence encoding processed extracellular human ActRIIB polypeptide is as follows (SEQ ID NO: 8). The sequence as shown provides an arginine at position 64, and may be modified to provide an alanine instead.

```
(SEQ ID NO: 8)
    1 GGGCGTGGGG AGGCTGAGAC ACGGGAGTGC
        ATCTACTACA ACGCCAACTG
  51 GGAGCTGGAG CGCACCAACC AGAGCGGCCT
        GGAGCGCTGC GAAGGCGAGC
101 AGGACAAGCG GCTGCACTGC TACGCCTCCT
        GGCGCAACAG CTCTGGCACC
151 ATCGAGCTCG TGAAGAAGGG CTGCTGGCTA
        GATGACTTCA ACTGCTACGA
201 TAGGCAGGAG TGTGTGGCCA CTGAGGAGAA
        CCCCCAGGTG TACTTCTGCT
251 GCTGTGAAGG CAACTTCTGC AACGAACGCT
        TCACTCATTT GCCAGAGGCT
301 GGGGGCCCGG AAGTCACGTA CGAGCCACCC
        CCGACAGCCC CCACC
```

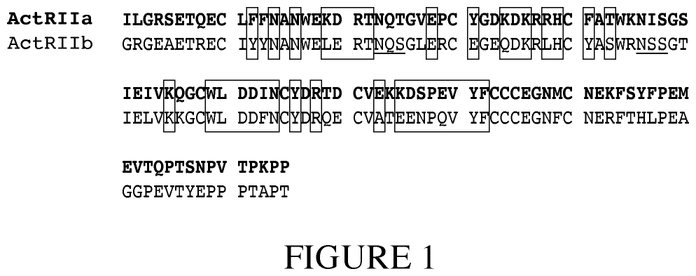

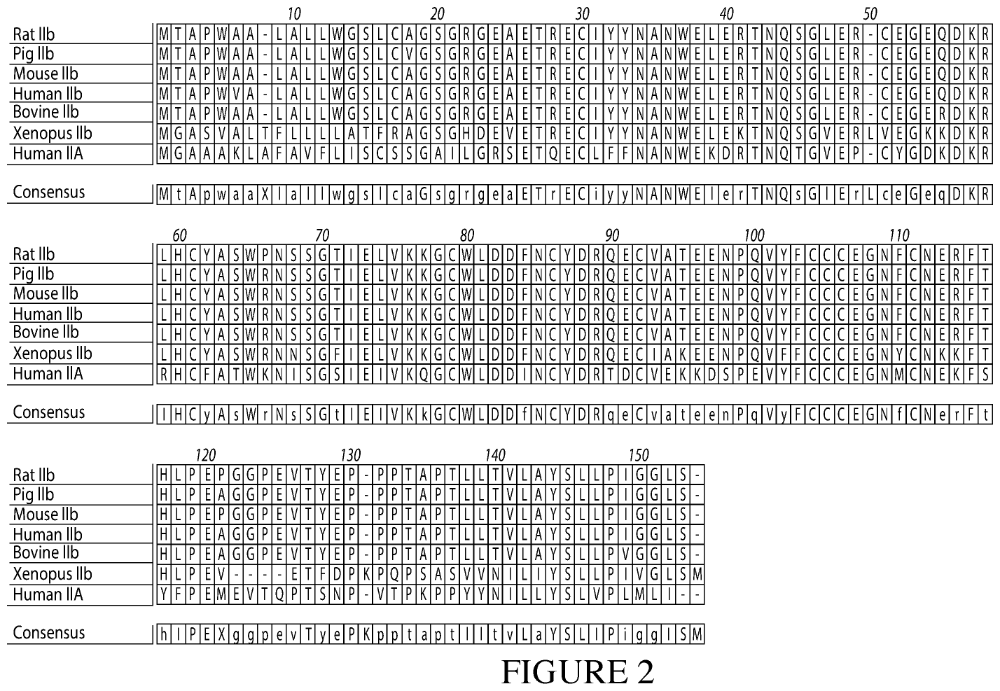

An alignment of the amino acid sequences of human ActRIIB soluble extracellular domain and human ActRIIA soluble extracellular domain are illustrated in FIG. 1. This alignment indicates amino acid residues within both receptors that are believed to directly contact ActRII ligands. FIG. 2 depicts a multiple-sequence alignment of various vertebrate ActRIIB proteins and human ActRIIA. From these alignments is it possible to predict key amino acid positions within the ligand-binding domain that are important for normal ActRII-ligand binding activities as well as to predict amino acid positions that are likely to be tolerant to substitution without significantly altering normal ActRII-ligand binding activities. ActRII proteins have been characterized in the art in terms of structural and functional characteristics, particularly with respect to ligand binding. See, e.g., Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709.605, 7,612,041, and 7,842,663.

For example, Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar but somewhat reduced activity relative to the wild-type, even though the proline knot region is disrupted. Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides and ActRIIB-based GDF traps ending at 128 (with respect to SEQ ID NO: 1) or later should retain ligand-binding activity. ActRIIB polypeptides and ActRIIB-based GDF traps ending at or between 19 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding. See, e.g., U.S. Pat. No. 7,842,663. This confirms that mutations in the region between the signal cleavage peptide and the cysteine cross-linked region, corresponding to amino acids 20-29, are well tolerated. In particular, ActRIIB polypeptides and ActRIIB-based GDF traps beginning at position 20, 21, 22, 23, and 24 (with respect to SEQ ID NO: 1) should retain general ligand-biding activity, and ActRIIB polypeptides and ActRIIB-based GDF traps beginning at positions 25, 26, 27, 28, and 29 (with respect to SEQ ID NO: 1) are also expected to retain ligand-biding activity. Data shown in, e.g., U.S. Pat. No. 7,842,663 demonstrates that, surprisingly, an ActRIIB construct beginning at 22, 23, 24, or 25 will have the most activity.

Taken together, an active portion (e.g., ligand-binding portion) of ActRIIB comprises amino acids 29-109 of SEQ ID NO: 1. Therefore ActRIIB polypeptides of the present disclosure may, for example, comprise an amino acid sequence that is at least 80%, 85%, 90%, 91%, 92%, 93%, 94%, 95%, 96%, 97%, 98%, 99%. or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., position 20, 21, 22, 23, 24, 25, 26, 27, 28, or 29) or 21-29 (e.g., position 21, 22, 23, 24, 25, 26, 27, 28. or 29) and end at a position from 119-134 (e.g., 119, 120, 121, 122, 123, 124, 125, 126, 127, 128, 129, 130, 131, 132, 133, or 134), 119-133 (e.g., 119, 120, 121, 122, 123, 124, 125, 126, 127, 128, 129, 130, 131, 132, or 133), 129-134 (e.g., 129, 130, 131, 132, 133, or 134), or 129-133 (e.g., 129, 130, 131, 132, or 133) of SEQ ID NO: 1. Other examples include constructs that begin at a position from 20-24 (e.g., 20, 21, 22, 23, or 24), 21-24 (e.g. 21, 22, 23, or 24), or 22-25 (e.g., 22, 22, 23, or 25) and end at a position from 109-134 (e.g., 109, 110, 1l 1, 112, 113, 114, 115, 116, 117, 118, 119, 120, 121, 122, 123, 124, 125, 126, 127, 128, 129, 130, 131, 132, 133, or 134), 119-134 (e.g., 119, 120, 121, 122, 123, 124, 125, 126, 127, 128, 129, 130, 131, 132, 133, or 134) or 129-134 (e.g., 129, 130, 131, 132, 133, or 134) of SEQ ID NO: 1. Variants within these ranges are also contemplated, particularly those having at least 80%, 85%, 90%, 91%, 92%, 93%, 94%, 95%, 96%, 97%, 98%, 99%, or 100% identity to the corresponding portion of SEQ ID NO: 1.

The disclosure includes the results of an analysis of composite ActRIIB structures, shown in FIG. 2, demonstrating that the ligand-binding pocket is defined, in part, by residues Y31, N33, N35, L38 through T41, E47, E50, Q53 through K55, L57, H58, Y60, S62, K74, W78 through N83, Y85, R87, Δ92, and E94 through F101. Additionally, ActRIIB is well-conserved across nearly all vertebrates, with large stretches of the extracellular domain conserved completely. Accordingly, comparisons of ActRIIB sequences from various vertebrate organisms provide insights into residues that may be altered. For example, R40 is a K in Xenopus, indicating that basic amino acids at this position will be tolerated. L46 is a valine in Xenopus ActRIIB, and so this position may be altered, and optionally may be altered to another hydrophobic residue, such as V, I or F, or a non-polar residue such as A. E52 is a K in Xenopus, indicating that this site may be tolerant of a wide variety of changes, including polar residues, such as E, D, K, R, H, S, T, P, G, Y and probably A. Q53 is R in bovine ActRIIB and K in Xenopus ActRIIB, and therefore amino acids including R, K, Q, N and H will be tolerated at this position. T93 is a K in Xenopus, indicating that a wide structural variation is tolerated at this position, with polar residues favored, such as S, K, R, E, D, H, G, P, G and Y. F108 is a Y in Xenopus, and therefore Y or other hydrophobic group, such as 1, V or L should be tolerated. E111 is K in Xenopus, indicating that charged residues will be tolerated at this position, including D, R, K and H, as well as Q and N. R112 is K in Xenopus, indicating that basic residues are tolerated at this position, including R and H. A at position 119 is relatively poorly conserved, and appears as P in rodents and V in Xenopus, thus essentially any amino acid should be tolerated at this position. The variations described herein may be combined in various ways. Additionally, the results of a mutagenesis program described in the art also confirms that there are amino acid positions in ActRIIB that are often beneficial to conserve. With respect to SEQ ID NO: 1, these include position 64 (basic amino acid), position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, in the ActRIIB polypeptides disclosed herein, the disclosure provides a framework of amino acids that may be conserved. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

Thus, a general formula for an ActRIIB polypeptide of the disclosure is one that comprises an amino acid sequence that is at least 80%, 85%, 90%, 91%, 92%, 93%, 94%, 95%, 96%, 97%, 98%, 99%, or 100% identical to amino acids 29-109 of SEQ ID NO: 1, optionally beginning at a position ranging from 20-24 (e.g., 20, 21, 22, 23, or 24) or 22-25 (e.g., 22, 23, 24, or 25) and ending at a position ranging from 129-134 (e.g., 129, 130, 131, 132, 133, or 134), and comprising no more than 1, 2, 5, 10 or 15 conservative amino acid changes in the ligand-binding pocket, and zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) actually improves ligand binding in the Δ64 background, and is thus expected to have no detrimental effect on ligand binding in the R64 background. See, e.g., U.S. Pat. No. 7,842,663. This change probably eliminates glycosylation at N65 in the Δ64 background, thus demonstrating that a significant change in this region is likely to be tolerated. While an R64A change is poorly tolerated, R64K is well-tolerated, and thus another basic residue, such as H may be tolerated at position 64. See, e.g., U.S. Pat. No. 7,842,663.

In certain embodiments. the disclosure relates to single-arm heteromultimers that comprise at least one ActRIIB polypeptide, which includes fragments, functional variants, and modified forms thereof. Preferably, ActRIIB polypeptides for use in accordance with inventions of the disclosure (e.g., single-arm heteromultimers comprising an ActRIIB polypeptide and uses thereof) are soluble (e.g., an extracellular domain of ActRIIB). In other preferred embodiments, ActRIIB polypeptides for use in accordance with the inventions of the disclosure bind to and/or inhibit (antagonize) activity (e.g., induction Smad signaling) of one or more TGF-beta superfamily ligands. In some embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115,116, 117, 118, 119, 120, 121, 122, 123, 124, 125, 126, 127, 128, 129, 130, 131, 132, 133, or 134) of SEQ ID NO: 1. In some embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical to a portion of ActRIIB beginning at a residue corresponding to amino acids 20-29 (e.g., beginning at amino acid 20, 21, 22, 23, 24, 25, 26, 27, 28, or 29) of SEQ ID NO: 1 and ending at a position corresponding to amino acids 109-134 (e.g., ending at amino acid 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1, wherein the position corresponding to L79 of SEQ ID NO: 1 is an acidic amino acid (i.e., a D or E amino acid residue). In certain preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1. In other preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (i.e., a D or E amino acid residue). In other preferred embodiments, single-arm heteromultimers of the disclosure comprise at least one ActRIIB polypeptide that comprises, consists, or consists essentially of an amino acid sequence that is at least 80%, 85%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is not an acidic amino acid (i.e., a D or E amino acid residue). In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, or 99% identical to the amino acid sequence of any one of SEQ ID NOs: 1, 2, 3, 4, 5, 6, 46, 48, 60, 61, 84, 86, 90, or 91. In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, 61, 84, 86, 90, or 91, wherein the position corresponding to L79 of SEQ ID NO: 1 is an acidic amino acid (i.e., a D or E amino acid residue). In some embodiments, single-arm heteromultimers of the disclosure comprise at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, 61, 84, 86, 90, or 91, wherein the position corresponding to L79 of SEQ ID NO: 1 is not an acidic amino acid (i.e., a D or E amino acid residue). In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, 61, 84, 86, 90, or 91. In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, 61, 84, 86, 90, or 91, wherein the position corresponding to L79 of SEQ ID NO: 1 is an acidic amino acid (i.e., a D or E amino acid residue). In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of any one of SEQ ID NOs: 1, 2, 3, 4, 5, 6, 46, 48, 60, 61, 84, 86, 90, or 91, wherein the position corresponding to L79 of SEQ ID NO: 1 is not an acidic amino acid (i.e., a D or E amino acid residue).

In some embodiments, single-arm heteromultimers of the disclosure comprise, consist, or consist essentially of at least one ActRIIB polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 83, wherein the position corresponding to L79 is an aspartic acid (D). The amino acid sequence for the truncated GDF trap ActRIIB(L79D 25-131) without the leader, hFc domain, and linker (SEQ ID NO: 83) is shown below. The aspartate substituted at position 79 in the native sequence is underlined and bolded, as is the glutamate revealed by sequencing to be the N-terminal residue in the mature fusion protein.

```
(SEQ ID NO: 83)
    1 ETRECIYYNA NWELERTNQS GLERCEGEQD
        KRLHCYASWR NSSGTIELVK
  51 KGCWDDDFNC YDRQECVATE ENPQVYFCCC
        EGNFCNERFT HLPEAGGPEV
111 TYEPPPT
```

In certain embodiments, the present disclosure relates to a protein complex comprising an ActRIIA polypeptide. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication No. WO 2006/012627, which is incorporated herein by reference in its entirety. Numbering of amino acids for all ActRIIA-related polypeptides described herein is based on the numbering of the human ActRIIA precursor protein sequence provided below (SEQ ID NO: 9), unless specifically designated otherwise.

The human ActRIIA precursor protein sequence is as follows:

(SEQ ID NO: 9)

| | |
|---|---|
| 1 | MGAAAKLAFA VFLISCSSGA ILGRSETQEC LFFNANWEKD |
| | RTNQTGVEPC |
| 51 | YGDKDKRRHC FATWKNISGS IEIVKQGCWL DDINCYDRTD |
| | CVEKKDSPEV |
| 101 | YFCCCEGNMC NEKFSYFPEM EVTQPTSNPV TPKPPYYNIL |
| | LYSLVPLMLI |
| 151 | AGIVICAFWV YRHHKMAYPP VLVPTQDPGL PPPSPLLGLK |
| | PLQLLEVKAR |
| 201 | GRFGCVWKAQ LLNEYVAVKI FPIQDKQSWQ NEYEVYSLPG |
| | MKHENILQFI |
| 251 | GAEKRGTSVD VDLWLITAFH EKGSLSDFLK ANVVSWNELC |
| | HIAETMARGL |
| 301 | AYLHEDIPGL KDGHKPAISH RDIKSKNVLL KNNLTACIAD |
| | FGLALKFEAG |
| 351 | KSAGDTHGQV GTRRYMAPEV LEGAINFQRD AFLRIDMYAM |
| | GLVLWELASR |
| 401 | CTAADGPVDE YMLPFEEEIG QHPSLEDMQE VVVHKKKRPV |
| | LRDYWQKHAG |
| 451 | MAMLCETIEE CWDHDAEARL SAGCVGERIT QMQRLTNIIT |
| | TEDIVTVVTM |
| 501 | VTNVDFPPKE SSL |

The signal peptide is indicated by a single underline: the extracellular domain is indicated in bold font; and the potential, endogenous N-linked glycosylation sites are indicated by a double underline.

The processed extracellular human ActRIIA polypeptide sequence is as follows:

(SEQ ID NO: 10)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by a single underline. The sequence with the “tail” deleted (a Δ15 sequence) is as follows:

(SEQ ID NO: 11)
ILGRSETQECLFENANWEKDRTNQTGVEPCYGDKDKRRHC
FATWKNISGSIEIVKQGCWLDDINCYDRTDCVEKKDSPEV
YFCCCEGNMCNEKFSYFPEM

A nucleic acid sequence encoding the human ActRIIA precursor protein is shown below (SEQ ID NO: 12), corresponding to nucleotides 159-1700 of Genbank Reference Sequence NM_001616.4. The signal sequence is underlined.

(SEQ ID NO: 12)

| | |
|---|---|
| 1 | ATGGGAGCTG CTGCAAAGTT GGCGTTTGCC GTCTTTCTTA |
| | TCTCCTGTTC |
| 51 | TTCAGGTGCT ATACTTGGTA GATCAGAAAC TCAGGAGTGT |
| | CTTTTCTTTA |
| 101 | ATGCTAATTG GGAAAAAGAC AGAACCAATC AAACTGGTGT |
| | TGAACCGTGT |
| 151 | TATGGTGACA AAGATAAACG GCGGCATTGT TTTGCTACCT |
| | GGAAGAATAT |
| 201 | TTCTGGTTCC ATTGAAATAG TGAAACAAGG TTGTTGGCTG |
| | GATGATATCA |
| 251 | ACTGCTATGA CAGGACTGAT TGTGTAGAAA AAAAAGACAG |
| | CCCTGAAGTA |
| 301 | TATTTTTGTT GCTGTGAGGG CAATATGTGT AATGAAAAGT |
| | TTTCTTATTT |
| 351 | TCCGGAGATG GAAGTCACAC AGCCCACTTC AAATCCAGTT |
| | ACACCTAAGC |
| 401 | CACCCTATTA CAACATCCTG CTCTATTCCT TGGTGCCACT |
| | TATGTTAATT |
| 451 | GCGGGGATTG TCATTTGTGC ATTTTGGGTG TACAGGCATC |
| | ACAAGATGGC |
| 501 | CTACCCTCCT GTACTTGTTC CAACTCAAGA CCCAGGACCA |
| | CCCCCACCTT |
| 551 | CTCCATTACT AGGTTTGAAA CCACTGCAGT TATTAGAAGT |
| | GAAAGCAAGG |
| 601 | GGAAGATTTG GTTGTGTCTG GAAAGCCCAG TTGCTTAACG |
| | AATATGTGGC |
| 651 | TGTCAAAATA TTTCCAATAC AGGACAAACA GTCATGGCAA |
| | AATGAATACG |
| 701 | AAGTCTACAG TTTGCCTGGA ATGAAGCATG AGAACATATT |
| | ACAGTTCATT |
| 751 | GGTGCAGAAA AACGAGGCAC CAGTGTTGAT GTGGATCTTT |
| | GGCTGATCAC |
| 801 | AGCATTTCAT GAAAAGGGTT CACTATCAGA CTTTCTTAAG |
| | GCTAATGTGG |
| 851 | TCTCTTGGAA TGAACTGTGT CATATTGCAG AAACCATGGC |
| | TAGAGGATTG |
| 901 | GCATATTTAC ATGAGGATAT ACCTGGCCTA AAAGATGGCC |
| | ACAAACCTGC |
| 951 | CATATCTCAC AGGGACATCA AAAGTAAAAA TGTGCTGTTG |
| | AAAAACAACC |
| 1001 | TGACAGCTTG CATTGCTGAC TTTGGGTTGG CCTTAAAATT |
| | TGAGGCTGGC |
| 1051 | AAGTCTGCAG GCGATACCCA TGGACAGGTT GGTACCCGGA |
| | GGTACATGGC |
| 1101 | TCCAGAGGTA TTAGAGGGTG CTATAAACTT CCAAAGGGAT |
| | GCATTTTTGA |
| 1151 | GGATAGATAT GTATGCCATG GGATTAGTCC TATGGGAACT |
| | GGCTTCTCGC |
| 1201 | TGTACTGCTG CAGATGGACC TGTAGATGAA TACATGTTGC |
| | CATTTGAGGA |
| 1251 | GGAAATTGGC CAGCATCCAT CTCTTGAAGA CATGCAGGAA |
| | GTTGTTGTGC |
| 1301 | ATAAAAAAAA GAGGCCTGTT TTAAGAGATT ATTGGCAGAA |
| | ACATGCTGGA |
| 1351 | ATGGCAATGC TCTGTGAAAC CATTGAAGAA TGTTGGGATC |
| | ACGACGCAGA |
| 1401 | AGCCAGGTTA TCAGCTGGAT GTGTAGGTGA AAGAATTACC |
| | CAGATGCAGA |
| 1451 | GACTAACAAA TATTATTACC ACAGAGGACA TTGTAACAGT |
| | GGTCACAATG |
| 1501 | GTGACAAATG TTGACTTTCC TCCCAAAGAA TCTAGTCTA |

The nucleic acid sequence encoding processed extracellular ActRIIA polypeptide is as follows:

```
(SEQ ID NO: 13)
    1 ATACTTGGTA GATCAGAAAC TCAGGAGTGT
        CTTTTCTTTA ATGCTAATTG
  51 GGAAAAAGAC AGAACCAATC AAACTGGTGT
        TGAACCGTGT TATGGTGACA
101 AAGATAAACG GCGGCATTGT TTTGCTACCT
        GGAAGAATAT TTCTGGTTCC
151 ATTGAAATAG TGAAACAAGG TTGTTGGCTG
        GATGATATCA ACTGCTATGA
201 CAGGACTGAT TGTGTAGAAA AAAAAGACAG
        CCCTGAAGTA TATTTTTGTT
251 GCTGTGAGGG CAATATGTGT AATGAAAAGT
        TTTCTTATTT TCCGGAGATG
301 GAAGTCACAC AGCCCACTTC AAATCCAGTT
        ACACCTAAGC CACCC
```

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 10, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

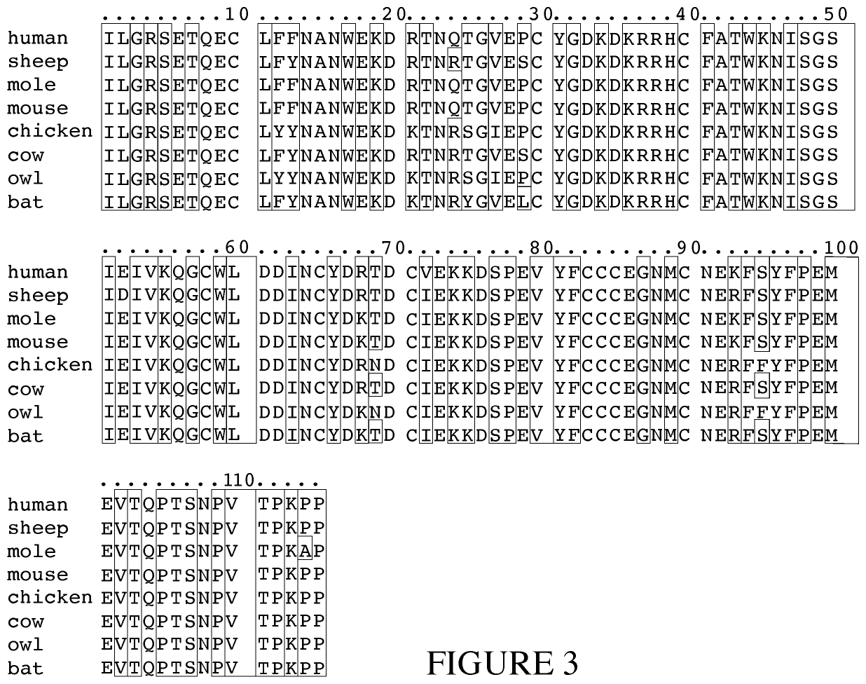

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly. from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain is Y in Ovis aries (SEQ ID NO: 76), Gallus gallus (SEQ ID NO: 79), Bos Taurus (SEQ ID NO: 80), Tyro alba (SEQ ID NO: 81), and Myotis davidii (SEQ ID NO: 82) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus galius and Tylo alba ActRIIA, indicating that this site may be tolerant of a wide variety of changes, including polar residues, such as E, D, K, R, H, S, T, P, G, Y, and probably hydrophobic residues such as L, 1, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

In certain embodiments, the disclosure relates to single-arm heteromultimers that comprise at least one ActRIIA polypeptide, which includes fragments, functional variants, and modified forms thereof. Preferably. ActRIIA polypeptides for use in accordance with inventions of the disclosure (e.g., single-arm heteromultimers comprising an ActRIIA polypeptide and uses thereof) are soluble (e.g., an extracellular domain of ActRIIA). In other preferred embodiments, ActRIIA polypeptides for use in accordance with the inventions of the disclosure bind to and/or inhibit (antagonize) activity (e.g., induction of Smad signaling) of one or more TGF-beta superfamily ligands. In some embodiments, single-arm heteromultimers of the disclosure comprise at least one ActRIIA polypeptide that is at least 70%, 75%, 80%, 85%, 90%, 91%, 92%, 93%, 94%, 95%, 96%, 97%, 98%, or 99% identical to the amino acid sequence of any one of SEQ ID NOs: 9, 10, 11, 55, 57, 58, 59, 88, or 89. In some embodiments, single-arm heteromultimers of the disclosure comprise, consist, or consist essentially of at least one ActRIIA polypeptide that is at least 70%, 75%, 80%, 85%, 90%, 91%, 92%, 93%, 94%, 95%, 97%, 98%, or 99% identical to the amino acid sequence of any one of SEQ ID NOs: 9, 10, 11, 55, 57, 58, 59, 88, or 89.

In some embodiments, the present disclosure contemplates making functional variants by modifying the structure of an ActRIIA or ActRIIB polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more ActRIIA or ActRIIB ligands including, for example, Activin A, activin B, GDF11, GDF8, GDF3, BMP5, BMP6, and BMP10.

In certain embodiments, the present disclosure contemplates specific mutations of an ActRIIA or ActRIIB polypeptide of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline: (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, single-arm ActRIIA heteromultimers or single-arm ActRIIB heteromultimers of the present disclosure for use in humans may be expressed in a mammalian cell line that provides proper glycosylation, such as HEK293 or CHO cell lines, although other mammalian expression cell lines are expected to be useful as well.

In certain embodiments, the present disclosure contemplates specific mutations of an ActRIIA or ActRIIB polypeptide of the disclosure. In some embodiments, one or more amino acid residues of a polypeptide of the present disclosure can be modified. In some embodiments, a modification is a glycosylated amino acid. In some embodiments, a modification is a PEGylated amino acid. In some embodiments, a modification is a farnesylated amino acid. In some embodiments, a modification is an acetylated amino acid. In some embodiments, a modification is a biotinylated amino acid. In some embodiments, a modification is an amino acid conjugated to a lipid moiety. In some embodiments, a modification is an amino acid conjugated to an organic derivatizing agent. In some embodiments, a first and/or a second polypeptide of the present disclosure comprises one or more amino acid modifications selected from: a glycosylated amino acid, a PEGylated amino acid, a famesylated amino acid, an acetylated amino acid, a biotinylated amino acid, an amino acid conjugated to a lipid moiety, and an amino acid conjugated to an organic derivatizing agent. In some embodiments, a first and/or second polypeptide is glycosylated and has a glycosylation pattern obtainable from expression of the first and/or second polypeptide in a CHO cell.

The present disclosure further contemplates a method of generating mutants, particularly sets of combinatorial mutants of an ActRIIA or ActRIIB polypeptide of the present disclosure, as well as truncation mutants. Pools of combinatorial mutants are especially useful for identifying ActRIIA or ActRIIB polypeptide sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, ActRIIA or ActRIIB polypeptide variants may be screened for ability to bind to an ActRIIA or ActRIIB ligand (e.g., activin A, activin B, GDF11, GDF8, GDF3, BMP5, BMP6, and BMP10), to prevent binding of an ActRIIA or ActRIIB ligand to an ActRIIA or ActRIIB polypeptide, and/or to interfere with signaling caused by an ActRIIA or ActRIIB ligand. In some embodiments, a heteromultimer of the present disclosure inhibits activity of one or more ActRIIA or ActRIIB ligands in a cell-based assay.

The activity of an ActRIIA or ActRIIB single-arm heteromultimer of the disclosure also may be tested in a cell-based or in vivo assay. For example, the effect of a single-arm heteromultimer on the expression of genes involved in a renal disease or condition (e.g., Alport syndrome, focal segmental glomerulosclerosis (FSGS), polycystic kidney disease, chronic kidney disease) may be assessed. This may, as needed, be performed in the presence of one or more recombinant ActRIIA or ActRIIB ligand proteins (e.g., activin A, activin B, GDF11, GDF8, GDF3, BMP5, BMP6, and BMP10), and cells may be transfected so as to produce single-arm ActRIIA heteromultimer or a single-arm ActRIIB heteromultimer, and optionally, an ActRIIA or ActRIIB ligand. Likewise, a single-arm heteromultimer of the disclosure may be administered to a mouse or other animal, and one or more measurements, such as albumin creatinine ratio (ACR), glomerular filtration rate (GFR), and/or blood urea nitrogen (BUN) may be assessed using art-recognized methods. Similarly, the activity of an ActRIIA or ActRIIB polypeptide or its variants may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference single-arm ActRIIA heteromultimer or a single-arm ActRIIB heteromultimer. Such variants, when expressed from recombinant DNA constructs, can be used in gene therapy protocols. Likewise, mutagenesis can give rise to variants which have extracellular half-lives dramatically different than the corresponding unmodified single-arm ActRIIA heteromultimer or single-arm ActRIIB heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system. can allow tighter control of recombinant polypeptide complex levels outside the cell. In an Fc fusion protein, mutations may be made in the linker (if any) and/or the Fc portion to alter the half-life of the single-arm ActRIIA heteromultimer or single-arm ActRIIB heteromultimer.

A combinatorial library may be produced by way of a degenerate library of genes encoding a library of polypeptides which each include at least a portion of potential ActRIIA or ActRIIB sequences. For instance, a mixture of synthetic oligonucleotides can be enzymatically ligated into gene sequences such that the degenerate set of potential ActRIIA or ActRIIB encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art. See, e.g., Narang, S A (1983) Tetrahedron 39:3; Itakura et al. (1981) Recombinant DNA, Proc. 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins. See, e.g., Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815.

Alternatively, other forms of mutagenesis can be utilized to generate a combinatorial library. For example, single-arm ActRIIA heteromultimers or single-arm ActRIIB heteromultimers of the disclosure can be generated and isolated from a library by screening using, for example, alanine scanning mutagenesis [see, e.g., Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [see, e.g., Gustin et al. (1993) Virology 193:653-660; and Brown et al. (1992) Mol. Cell Biol. 12:2644-2652; McKnight et al. (1982) Science 232:316], by saturation mutagenesis [see, e.g., Meyers et al., (1986) Science 232:613]; by PCR mutagenesis [see, e.g., Leung et al. (1989) Method Cell Mol Biol 1:11-19]; or by random mutagenesis, including chemical mutagenesis [see, e.g., Miller et al. (1992) A Short Course in Bacterial Genetics, CSHL Press, Cold Spring Harbor, N.Y.; and Greener et al. (1994) Strategies in Mol Biol 7:32-34]. Linker scanning mutagenesis, particularly in a combinatorial setting, is an attractive method for identifying truncated (bioactive) forms of ActRIIA or ActRIIB polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of single-arm ActRIIA heteromultimers or single-arm ActRIIB heteromultimers of the disclosure. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include binding assays and/or cell-signaling assays for ActRIIA or ActRIIB ligands (e.g., Activin A, activin B, GDF11, GDF8, GDF3, BMP5, BMP6, and BMP10).

In certain embodiments, single-arm ActRIIA heteromultimers or single-arm ActRIIB heteromultimers of the disclosure may further comprise post-translational modifications in addition to any that are naturally present in the ActRIIA or ActRIIB polypeptide. Such modifications include, but are not limited to, acetylation, carboxylation, glycosylation, phosphorylation, lipidation, and acylation. As a result, the ActRIIA or ActRIIB single-arm heteromultimer may comprise non-amino acid elements, such as polyethylene glycols, lipids, polysaccharide or monosaccharide, and phosphates. Effects of such non-amino acid elements on the functionality of a single-arm heteromultimer may be tested as described herein for other single-arm heteromultimer variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the ActRIIA or ActRIIB polypeptide.

In certain aspects, the polypeptides disclosed herein may form protein heteromultimers comprising at least one ActRIIA or ActRIIB polypeptide associated, covalently or non-covalently, with at least one polypeptide comprising a complementary member of an interaction pair. Preferably, polypeptides disclosed herein form single-arm heterodimers, although higher order heteromultimers are also included such as, but not limited to, heterotrimers, heterotetramers, and further oligomeric structures. In some embodiments, ActRIIA or ActRIIB polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a single-arm polypeptide (e.g., a fusion polypeptide comprising an ActRIIA or ActRIIB polypeptide) and a complementary member of an interaction pair to promote heteromultimer formation (e.g., heterodimer formation), and optionally hinders or otherwise disfavors homomultimer formation (e.g., homodimer formation), thereby increasing the yield of desired heteromultimer.

Many methods known in the art can be used to generate single-arm ActRIIA heteromultimers or single-arm ActRIIB heteromultimers of the disclosure. For example, non-naturally occurring disulfide bonds may be constructed by replacing on a first polypeptide (e.g., a fusion polypeptide comprising an ActRIIA or ActRIIB polypeptide) a naturally occurring amino acid with a free thiol-containing residue, such as cysteine, such that the free thiol interacts with another free thiol-containing residue on a second polypeptide (e.g., a complementary member of an interaction pair) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S.20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form heteromultimers comprising a first polypeptide covalently or non-covalently associated with a second polypeptide, wherein the first polypeptide comprises the amino acid sequence of an ActRIIA or ActRIIB polypeptide and the amino acid sequence of a first member of an interaction pair; and the second polypeptide comprises the amino acid sequence of a second member of an interaction pair. The interaction pair may be any two polypeptide sequences that interact to form a heteromultimer, particularly a heterodimer, although operative embodiments may also employ an interaction pair that can form a homodimeric complex. One member of the interaction pair may be fused to an ActRIIA or ActRIIB polypeptide as described herein, including for example, a polypeptide sequence comprising, consisting essentially of, or consisting of an amino acid sequence that is at least 80%, 85%, 90%, 91%, 92%, 93%, 94%, 95%, 96%, 97%, 98%, 99%, or 100% identical to the sequence of any one of SEQ ID NOs: 1, 2, 3, 4, 5, 6, 9, 10, 11. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric interaction-pair. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer interaction-pair or a heterodimeric action-pair. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides heteromultimer fusion proteins comprising at least one ActRIIA or ActRIIB polypeptide fused to a polypeptide. In some embodiments, the present disclosure provides heteromultimer fusion proteins comprising at least one ActRIIA or ActRIIB polypeptide fused to a polypeptide comprising a constant region of an immunoglobulin, such as a CH1, CH2, or CH3 domain of an immunoglobulin or an Fc domain. Fc domains derived from human IgG1, IgG2, IgG3, and IgG4 are provided herein. In some embodiments, the first constant region from an IgG heavy chain is a first immunoglobulin Fc domain. In some embodiments, a second constant region from an IgG heavy chain is a first immunoglobulin Fc domain. In some embodiments, Fc domains are derived from the constant region from a human IgG1, IgG2, IgG3, or IgG4 heavy chain. In some embodiments, Fc domains comprise the constant region from a human IgG1. In some embodiments, Fe domains comprise the constant region from a human IgG2. In some embodiments, Fc domains comprise the constant region from a human IgG3. In some embodiments, Fc domains comprise the constant region from a human IgG4. In some embodiments, the amino acid sequence of an immunoglobulin or an Fc domain may optionally be provided with the C-terminal lysine (K) removed (e.g., SEQ ID NOs: 85 and 87).

Other mutations are known that decrease either CDC or ADCC activity, and collectively, any of these variants are included in the disclosure and may be used as advantageous components of a single-arm heteromultimer fusion protein of the disclosure. Optionally, the IgG1 Fc domain of SEQ ID NO: 22 has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

An example of a native amino acid sequence that may be used for the Fc portion of human IgG1 (G1Fc) is shown below (SEQ ID NO: 22). Dotted underline indicates the hinge region, and solid underline indicates positions with naturally occurring variants. In part, the disclosure provides polypeptides comprising amino acid sequences with 80%, 85%, 90%, 91%, 92%, 93%, 94%, 95%, 96%, 97%, 98%, or 99% identity to SEQ ID NO: 22. Naturally occurring variants in G1Fc would include E134D and M136L according to the numbering system used in SEQ ID NO: 22 (see UniProt P01857).

(SEQ ID NO: 22)

| | |
|---|---|
| 1 | [symbol] AP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSREEMTKNQ |
| | VSLTCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGK |

An example of a native amino acid sequence that may be used for the Fc portion of human IgG2 (G2Fc) is shown below (SEQ ID NO: 23). Dotted underline indicates the hinge region and double underline indicates positions where there are database conflicts in the sequence (according to UniProt P01859). In part, the disclosure provides polypeptides comprising amino acid sequences with 80%, 85%, 90%, 91%, 92%, 93%, 94%, 95%, 96%, 97%, 98%, or 99% identity to SEQ ID NO: 23.

(SEQ ID NO: 23)

| | |
|---|---|
| 1 | [symbol] APP VAGPSVFLFP PKPKDTLMIS RTPEVTCVVV |
| | DVSHEDPEVQ |
| 51 | FNWYVDGVEV HNAKTKPREE QFNSTFRVVS VLTVVHQDWL |
| | NGKEYKCKVS |
| 101 | NKGLPAPIEK TISKTKGQPR EPQVYTLPPS REEMTKNQVS |
| | LTCLVKGFYP |
| 151 | SDIAVEWESN GQPENNYKTT PPMLDSDGSF FLYSKLTVDK |
| | SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 24) contains a short hinge region consisting of a single 15-residue segment, whereas the second G3Fc sequence (SEQ ID NO: 25) contains a full-length hinge region. In each case, dotted underline indicates the hinge region, and solid underline indicates positions with naturally occurring variants according to UniProt P01859. In part, the disclosure provides polypeptides comprising amino acid sequences with 80%, 85%, 90%, 91%, 92%, 93%, 94%, 95%, 96%, 97%, 98%, or 99% identity to SEQ ID NOs: 24 and 25.

(SEQ ID NO: 24)

| | |
|---|---|
| 1 | [symbol] [symbol] APELL GGPSVFLFPP KPKDTLMISR |
| | TPEVTCVVVD |
| 51 | VSHEDPEVQF KWYVDGVEVH NAKTKPREEQ YNSTFRVVSV |
| | LTVLHQDWLN |
| 101 | GKEYKCKVSN KALPAPIERT ISKTKGQPRE PQVYTLPPSR |
| | EEMTKNQVSL |
| 151 | TCLVKGFYPS DIAVEWESSG QPENNYNTTP PMLDSDGSFF |
| | LYSKLTVDKS |
| 201 | RWQQGNIFSC SVMHEALHNR FTQKSLSLSP GK |

(SEQ ID NO: 25)

| | |
|---|---|
| 1 | [symbol] [symbol] [symbol] [symbol] |
| | [symbol] |
| 51 | [symbol] [symbol] APELLGGP SVFLFPPKPK DTLMISRTPE |
| | VTCVVVDVSH |
| 101 | EDPEVQFKWY VDGVEVHNAK TKPREEQYNS TFRVVSVLTV |
| | LHQDWLNGKE |
| 151 | YKCKVSNKAL PAPIEKTISK TKGQPREPQV YTLPPSREEM |
| | TKNQVSLTCL |
| 201 | VKGFYPSDIA VEWESSGQPE NNYNTTPPML DSDGSFFLYS |
| | KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 24, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 26). Dotted underline indicates the hinge region. In part, the disclosure provides polypeptides comprising amino acid sequences with 80%, 85% 90%, 91%, 92%, 93%, 94%, 95%, 96%, 97%, 98%, or 99% identity to SE ID NO: 26.

(SEQ ID NO: 26)

| | |
|---|---|
| 1 | [symbol] [symbol] APEFLGGP SVFLEPPKPK DTLMISRTPE |
| | VTCVVVDVSQ |
| 51 | EDPEVQFNWY VDGVEVHNAK TKPREEQFNS TYRVVSVLTV |
| | LHQDWLNGKE |
| 101 | YKCKVSNKGL PSSIEKTISK AKGQPREPQV YTLPPSQEEM |
| | TKNQVSLTCL |
| 151 | VKGFYPSDIA VEWESNGQPE NNYKTTPPVI DSDGSFFLYS |
| | RLTVDKSRWQ |
| 201 | EGNVFSCSVM HEALHNHYTQ KSLSLSLGK |

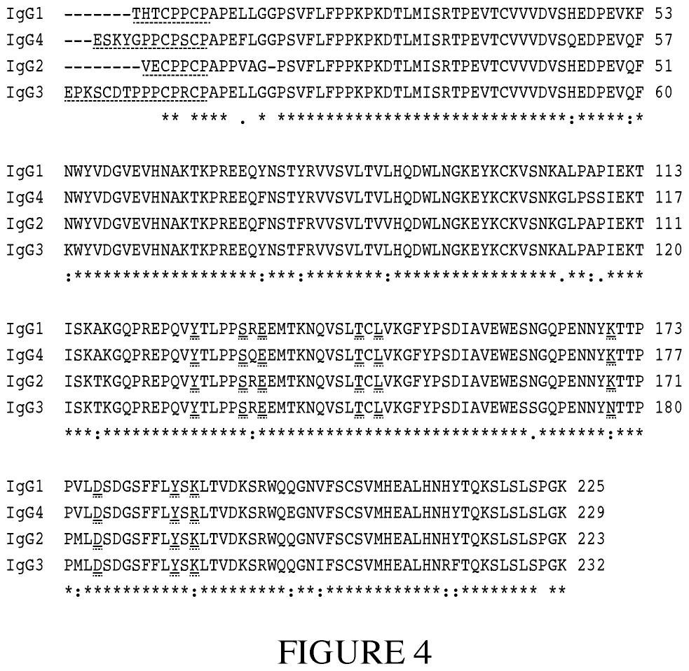

A variety of engineered mutations in the Fc domain are presented herein with respect to the G1Fc sequence (SEQ ID NO: 22), and analogous mutations in G2Fc, G3Fc. and G4Fc can be derived from their alignment with G1Fc in FIG. 4. Due to unequal hinge lengths, analogous Fc positions based on isotype alignment (FIG. 4) possess different amino acid numbers in SEQ ID NOs: 22, 23, 24, and 26. It can also be appreciated that a given amino acid position in an immunoglobulin sequence consisting of hinge, C_{H}2, and C_{H}3 regions (e.g., SEQ ID NOs: 22, 23, 24, and 26) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 22). the human IgG1 heavy chain constant domain (Uniprot P01857), and the human IgG11 heavy chain is as follows.

Correspondence of C_{H}3 Positions in Different Numbering Systems

| G1Fc | | |
|---|---|---|
| (Numbering | IgG1 heavy chain | IgG1 heavy chain |
| begins at first | constant domain | (EU numbering |
| threonine in | (Numbering begins | scheme of Kabat |
| hinge region) | at C_{H}1) | et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5th ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681: Davis et al (2010) Protein Eng Des Sel 23:195-202; Gunasekaran et al (2010); 285:19637-19646: Wranik et al (2012) J Biol Chem 287:43331-43339; U.S. Pat. No. 5,932,448; WO 1993/011162; WO 2009/089004, and WO 2011/034605.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged, and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative: K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations
to Enhance Heterodimer Formation

| | | Interacting | Corresponding |
|---|---|---|---|
| Position in | Mutation in | position | mutation in |
| first chain | first chain | in second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments. one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the ActRIIA or ActRIIB polypeptide of the construct, with or without an optional linker, to generate an ActRIIA or ActRIIB fusion polypeptide This single chain can be co-expressed in a cell of choice along with the Fc sequence complementary to the first Fc to favor generation of the desired multichain construct (e.g., a single-arm ActRIIA heteromultimer or single-arm ActRIIB heteromultimer). In this example based on electrostatic steering, SEQ ID NO: 14 [human G1Fc(E134K/D177K)] and SEQ ID NO: 15 [human G1Fc(K170D/K187D)] are examples of complementary Fc sequences in which the engineered amino acid substitutions are double underlined, and the ActRIIA or ActRIIB polypeptide of the construct can be fused to either SEQ ID NO: 14 or SEQ ID NO: 15, but not both. Given the high degree of amino acid sequence identity between native hG1Fc, native hG2Fc. native hG3Fc, and native hG4Fc, it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 14 and 15).

(SEQ ID NO: 14)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSRKEMTKNQ |
| | VSLTCLVKGF |
| 151 | YPSDIAVEWE SNGQPENNYK TTPPVLKSDG SFFLYSKLTV |
| | DKSRWQQGNV |
| 201 | FSCSVMHEAL HNHYTQKSLS LSPGK |

(SEQ ID NO: 15)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSREEMTKNQ |
| | VSLTCLVKGF |
| 151 | YPSDIAVEWE SNGQPENNYD TTPPVLDSDG SFFLYSDLTV |
| | DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the ActRIIA or ActRIIB polypeptide of the construct, with or without an optional linker, to generate a single-arm ActRIIB heteromultimer fusion construct or a single-arm ActRIIB heteromultimer fusion construct. This single chain can be co-expressed in a cell of choice along with the Fc sequence complementary to the first Fc to favor generation of the desired multichain construct (e.g., a single-arm ActRIIA heteromultimer or a single-arm ActRIIB heteromultimer). In this example based on knobs-into-holes pairing, SEQ ID NO: 16 [human G1Fc(T144Y)] and SEQ ID NO: 17 [human G1Fc(Y185T)] are examples of complementary Fc sequences in which the engineered amino acid substitutions are double underlined, and the ActRIIA or ActRIIB polypeptide of the construct can be fused to either SEQ ID NO: 16 or SEQ ID NO: 17, but not both. Given the high degree of amino acid sequence identity between native hG1Fc. native hG2Fc, native hG3Fc, and native hG4Fc. it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 16 and 17).

(SEQ ID NO: 16)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP P3REEMTKNQ |
| | VSLYCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGK |

(SEQ ID NO: 17)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSREEMTKNQ |
| | VSLTCLVKGE |
| 151 | YPSDIAVEWE SNGQPENNYK TTPPVLDSDG SFFLTSKLTV |
| | DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 18 [hG1Fc(S132C/T144W)] and SEQ ID NO: 19 [hG1Fc(Y127C/T144S/L146A/Y185V)]. The engineered amino acid substitutions in these sequences are double underlined, and the ActRIIA or ActRIIB polypeptide of the construct can be fused to either SEQ ID NO: 18 or SEQ ID NO: 19, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 4) will generate complementary Fc pairs which may be used instead of the complementary hG1Fc pair below (SEQ ID NOs: 18 and 19).

(SEQ ID NO: 18)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PCREEMTKNQ |
| | VSLWCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGK |

(SEQ ID NO: 19)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVCTLP PSREEMTKNQ |
| | VSLSCAVKGF |
| 151 | YPSDIAVEWE SNGQPENNYK TTPPVLDSDG SFFLVSKLTV |
| | DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to the ActRIIA or ActRIIB polypeptide of the construct, with or without an optional linker, to generate a single-arm ActRIIA heteromultimer fusion construct or a single-arm ActRIIB heteromultimer construct. This single chain can be co-expressed in a cell of choice along with the Fc sequence complementary to the first Fc to favor generation of the desired multichain construct. In this example based on SEEDbody (Sb) pairing, SEQ ID NO: 20 [hG1Fc(Sb_{AG})] and SEQ ID NO: 21 [hG1Fc(Sb_{GA})] are examples of complementary IgG Fc sequences in which the engineered amino acid substitutions from IgA Fc are double underlined, and the ActRIIA or ActRIIB polypeptide of the construct can be fused to either SEQ ID NO: 20 or SEQ ID NO: 21, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc. or hG4Fc (see FIG. 4) will generate an Fc monomer which may be used in the complementary IgG-IgA pair below (SEQ ID NOs: 20 and 21).

(SEQ ID NO: 20)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PFRPEVHLL PPSREEMTKNQ |
| | VSLTCLARGF |
| 151 | YPKDIAVEWE SNGQPENNYK TTPSRQEPSQ GTTTFAVTSK |
| | LTVDKSRWQQ |
| 201 | GNVFSCSVMH EALHNHYTQK TISLSPGK |

(SEQ ID NO: 21)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PPSEELALNE |
| | LVTLTCLVKG |
| 151 | FYPSDIAVEW ESNGQELPRE KYLTWAPVLD SDGSFFLYSI |
| | LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to the ActRIIA or ActRIIB polypeptide of the construct, with or without an optional linker, to generate a single-arm ActRIIA heteromultimer fusion construct or a single-arm ActRIIB heteromultimer fusion construct. This single chain can be co-expressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 27 [hG1Fc-Ap1 (acidic)] and SEQ ID NO: 28 [hG1Fc-Bp1 (basic)] are examples of complementary IgG Fc sequences in which the engineered complimentary leucine zipper sequences are underlined, and the ActRIIA or ActRIIB polypeptide of the construct can be fused to either SEQ ID NO: 27 or SEQ ID NO: 28, but not both. Given the high degree of amino acid sequence identity between native hG1Fc, native hG2Fc, native hG3Fc. and native hG4Fc, it can be appreciated that leucine zipper-forming sequences attached, with or without an optional linker, to hG1Fc, hG2Fc, hG3Fc, or hG4Fc (see FIG. 4) will generate an Fc monomer which may be used in the complementary leucine zipper-forming pair below (SEQ ID NOs: 27 and 28).

(SEQ ID NO: 27)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTTSKAKGQ PREPQVYTLP PSREEMTKNQ |
| | VSLTCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGKGGSAQ LEKELQALEK |
| | ENAQLEWELQ |
| 251 | ALEKELAQGA T |

(SEQ ID NO: 28)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGO PREPQVYTLP PSREEMTKNQ |
| | VSLTCLVKGF |
| 151 | YPSDIAVEWE SNGQPENNYK TTPPVLDSDG SFFLYSKLTV |
| | DKSRWOQGNV |
| 201 | FSCSVMHEAL HNHYTQKSLS LSPGKGGSAQ LKKKLQALKK |
| | KNAQLKWKLQ |
| 251 | ALKKKLAQGA T |

It is understood that different elements of the fusion proteins (e.g., immunoglobulin Fc fusion proteins) may be arranged in any manner that is consistent with desired functionality. For example, an ActRIIA or ActRIIB polypeptide domain may be placed C-terminal to a heterologous domain, or alternatively. a heterologous domain may be placed C-terminal to an ActRIIA or ActRIIB polypeptide domain. The ActRIIA or ActRIIB polypeptide domain and the heterologous domain need not be adjacent in a fusion protein, and additional domains or amino acid sequences may be included C- or N-terminal to either domain or between the domains. For example, a single-arm ActRIIA heteromultimer fusion construct or single-arm ActRIIB heteromultimer fusion construct may comprise an amino acid sequence as set forth in the formula A-B-C. The B portion corresponds to an ActRIIA or ActRIIB polypeptide domain. The A and C portions may be independently zero, one, or more than one amino acid, and both the A and C portions when present are heterologous to B. The A and/or C portions may be attached to the B portion via a linker sequence. In certain embodiments, an ActRIIA or ActRIIB fusion polypeptide comprises an amino acid sequence as set forth in the formula A-B-C, wherein A is a leader (signal) sequence. B consists of an ActRIIA or ActRIIB polypeptide domain, and C is a polypeptide portion that enhances one or more of in vivo stability, in vivo half-life, uptake/administration, tissue localization or distribution, formation of protein complexes, and/or purification. In certain embodiments, an ActRIIA or ActRIIB fusion polypeptide comprises an amino acid sequence as set forth in the formula A-B-C, wherein A is a TPA leader sequence, B consists of an ActRIIA or ActRIIB polypeptide domain, and C is an immunoglobulin Fc domain. Preferred fusion polypeptides comprise the amino acid sequence set forth in any one of SEQ ID NOs: 46, 48, 55, 57, 58, 59, 60, 61, 84, 86, 88, 89, 90, or 91.

In some embodiments, single-arm ActRIIA heteromultimers or single-arm ActRIIB heteromultimers of the present disclosure further comprise one or more heterologous portions (domains) so as to confer a desired property. For example, some fusion domains are particularly useful for isolation of the fusion proteins by affinity chromatography. Well-known examples of such fusion domains include, but are not limited to, polyhistidine. Glu-Glu, glutathione S-transferase (GST), thioredoxin, protein A, protein G. an immunoglobulin heavy-chain constant region (Fc), maltose binding protein (MBP), or human serum albumin. For the purpose of affinity purification. relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ligand trap polypeptides. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagluttinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation.

In certain embodiments, ActRIIA or ActRIIB polypeptides of the present disclosure comprise one or more modifications that are capable of stabilizing the polypeptides. For example, such modifications enhance the in vitro half-life of the polypeptides, enhance circulatory half-life of the polypeptides, and/or reduce proteolytic degradation of the polypeptides. Such stabilizing modifications include, but are not limited to, fusion polypeptides (including, for example, fusion polypeptides comprising an ActRIIA or ActRIIB polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion polypeptides, but also includes nonproteinaceous modifications such as a carbohydrate moiety, or nonproteinaceous moiety, such as polyethylene glycol.

In preferred embodiments, single-arm ActRIIA heteromultimers or single-arm ActRIIB heteromultimers to be used in accordance with the methods described herein are isolated heteromultimers. As used herein, an isolated protein (e.g., heteromultimer) or polypeptide (e.g., heteromultimer) is one which has been separated from a component of its natural environment. In some embodiments, a single-arm heteromultimer of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [See, e.g., Flatman et al., (2007) J. Chromatogr. B 848:79-87].

In certain embodiments, ActRIIA or ActRIIB polypeptides, as well as single-arm heteromultimers thereof, of the disclosure, can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.). Synthetic Peptides: A User's Guide, W. H. Freeman and Company, New York (1992). In addition, automated peptide synthesizers are commercially available (see, e.g., Advanced ChemTech Model 396; Milligen/Biosearch 9600). Alternatively, the polypeptides and complexes of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRIIA or ActRIIB polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites.

In some embodiments, a single-arm ActRIIB heteromultimer of the present disclosure comprises a first polypeptide covalently or non-covalently associated with a second polypeptide, wherein the first polypeptide comprises the amino acid sequence of a first member of an interaction pair and the amino acid sequence of ActRIIB; and the second polypeptide comprises the amino acid sequence of a second member of the interaction pair, and wherein the second polypeptide does not comprise ActRIIB.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID NOs: 1, 2, 3, 4, 5, and 6; or at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 1. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 2. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 3. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 4. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 5. In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 6.

In some embodiments, the single-arm ActRIIB heteromultimer comprises, consists, or consists essentially of an amino acid sequence that is at least 70%, 80%, 85%, 90%, 91%, 92%, 93%, 94% 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1 In some embodiments, the single-arm ActRIIB heteromultimer does not comprise an acidic amino acid at the position corresponding to L79 of SEQ ID NO: 1. In some embodiments, the single-arm ActRIIB heteromultimer does not comprise an aspartic acid (D) at the position corresponding to L79 of SEQ ID NO: 1.

In some embodiments, the single-arm ActRIIB heteromultimer comprises the amino acid sequence of SEQ ID NO: 2. In some embodiments, the single-arm ActRIIB heteromultimer consists of the amino acid sequence of SEQ ID NO: 2. In some embodiments, the single-arm ActRIIB heteromultimer consists essentially of the amino acid sequence of SEQ ID NO: 2.

In some embodiments, the single-arm ActRIIB heteromultimer comprises the amino acid sequence of SEQ ID NO: 3. In some embodiments, the single-arm ActRIIB heteromultimer consists of the amino acid sequence of SEQ ID NO: 3. In some embodiments, the single-arm ActRIIB heteromultimer consists essentially of the amino acid sequence of SEQ ID NO: 3.

In some embodiments, the single-arm ActRIIB heteromultimer comprises the amino acid sequence of SEQ ID NO: 5. In some embodiments, the single-arm ActRIIB heteromultimer consists of the amino acid sequence of SEQ ID NO: 5. In some embodiments, the single-arm ActRIIB heteromultimer consists essentially of the amino acid sequence of SEQ ID NO: 5.

In some embodiments, the single-arm ActRIIB heteromultimer comprises the amino acid sequence of SEQ ID NO: 6. In some embodiments, the single-arm ActRIIB heteromultimer consists of the amino acid sequence of SEQ ID NO: 6. In some embodiments, the single-arm ActRIIB heteromultimer consists essentially of the amino acid sequence of SEQ ID NO: 6.

In some embodiments, the single-arm ActRIIB heteromultimer comprises the amino acid sequence of SEQ ID NO: 48. In some embodiments, the single-arm ActRIIB heteromultimer consists of the amino acid sequence of SEQ ID NO: 48. In some embodiments, the single-arm ActRIIB heteromultimer consists essentially of the amino acid sequence of SEQ ID NO: 48.

In some embodiments, the single-arm ActRIIB heteromultimer comprises the amino acid sequence of SEQ ID NO: 84. In some embodiments, the single-arm ActRIIB heteromultimer consists of the amino acid sequence of SEQ ID NO: 84. In some embodiments, the single-arm ActRIIB heteromultimer consists essentially of the amino acid sequence of SEQ ID NO: 84.

In some embodiments, the single-arm ActRIIB heteromultimer comprises the amino acid sequence of SEQ ID NO: 61. In some embodiments, the single-arm ActRIIB heteromultimer consists of the amino acid sequence of SEQ ID NO: 61. In some embodiments, the single-arm ActRIIB heteromultimer consists essentially of the amino acid sequence of SEQ ID NO: 61.

In some embodiments, the single-arm ActRIIB heteromultimer comprises the amino acid sequence of SEQ ID NO: 86. In some embodiments, the single-arm ActRIIB heteromultimer consists of the amino acid sequence of SEQ ID NO: 86. In some embodiments, the single-arm ActRIIB heteromultimer consists essentially of the amino acid sequence of SEQ ID NO: 86.

In some embodiments, the single-arm ActRIIB heteromultimer comprises the amino acid sequence of SEQ ID NO: 90. In some embodiments, the single-arm ActRIIB heteromultimer consists of the amino acid sequence of SEQ ID NO: 90. In some embodiments, the single-arm ActRIIB heteromultimer consists essentially of the amino acid sequence of SEQ ID NO: 90.

In some embodiments, the single-arm ActRIIB heteromultimer comprises the amino acid sequence of SEQ ID NO: 91. In some embodiments, the single-arm ActRIIB heteromultimer consists of the amino acid sequence of SEQ ID NO: 91. In some embodiments, the single-arm ActRIIB heteromultimer consists essentially of the amino acid sequence of SEQ ID NO: 91.

In some embodiments, a single-arm ActRIIA heteromultimer of the present disclosure comprises a first polypeptide covalently or non-covalently associated with a second polypeptide, wherein the first polypeptide comprises the amino acid sequence of a first member of an interaction pair and the amino acid sequence of ActRIIA; and the second polypeptide comprises the amino acid sequence of a second member of the interaction pair, and wherein the second polypeptide does not comprise ActRIIA.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of any of SEQ ID Nos: 9, 10, and 11; or at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 9. In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 10. In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to the sequence of SEQ ID NO: 11.

In some embodiments, the single-arm ActRIIA heteromultimer comprises, consists, or consists essentially of an amino acid sequence that is at least 70%, 80%, 85%, 90%, 91%, 92%, 93%, 94% 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

In some embodiments, the single-arm ActRIIA heteromultimer comprises the amino acid sequence of SEQ ID NO: 10. In some embodiments, the single-arm ActRIIA heteromultimer consists of the amino acid sequence of SEQ ID NO: 10. In some embodiments, the single-arm ActRIIA heteromultimer consists essentially of the amino acid sequence of SEQ ID NO: 10.

In some embodiments. the single-arm ActRIIA heteromultimer comprises the amino acid sequence of SEQ ID NO: 11. In some embodiments, the single-arm ActRIIA heteromultimer consists of the amino acid sequence of SEQ ID NO: 11. In some embodiments, the single-arm ActRIIA heteromultimer consists essentially of the amino acid sequence of SEQ ID NO: 11.

In some embodiments, the single-arm ActRIIA heteromultimer comprises the amino acid sequence of SEQ ID NO: 57. In some embodiments, the single-arm ActRIIA heteromultimer consists of the amino acid sequence of SEQ ID NO: 57. In some embodiments, the single-arm ActRIIA heteromultimer consists essentially of the amino acid sequence of SEQ ID NO: 57.

In some embodiments, the single-arm ActRIIA heteromultimer comprises the amino acid sequence of SEQ ID NO: 88. In some embodiments, the single-arm ActRIIA heteromultimer consists of the amino acid sequence of SEQ ID NO: 88. In some embodiments, the single-arm ActRIIA heteromultimer consists essentially of the amino acid sequence of SEQ ID NO: 88.

In some embodiments, the single-arm ActRIIA heteromultimer comprises the amino acid sequence of SEQ ID NO: 59. In some embodiments, the single-arm ActRIIA heteromultimer consists of the amino acid sequence of SEQ ID NO: 59. In some embodiments, the single-arm ActRIIA heteromultimer consists essentially of the amino acid sequence of SEQ ID NO: 59.

In some embodiments, the single-arm ActRIIA heteromultimer comprises the amino acid sequence of SEQ ID NO: 89. In some embodiments, the single-arm ActRIIA heteromultimer consists of the amino acid sequence of SEQ ID NO: 89. In some embodiments, the single-arm ActRIIA heteromultimer consists essentially of the amino acid sequence of SEQ ID NO: 89.

In some embodiments, the single-arm heteromultimer is a heterodimer. In some embodiments, the first member of an interaction pair comprises a first constant region from an IgG heavy chain. In some embodiments, the first constant region from an IgG heavy chain is a first immunoglobulin Fc domain. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 14-28.

In some embodiments. the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 14. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 15. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 16. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 17. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 18. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 19. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 20. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 21. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 22. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 23. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 24. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 25. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 26. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 27. In some embodiments, the first constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 28.

In some embodiments, the second member of an interaction pair comprises a second constant region from an IgG heavy chain. In some embodiments, the second constant region from an IgG heavy chain is a first immunoglobulin Fc domain. In some embodiments, the second constant region from an IgG heavy chain comprises an amino acid sequence that is at least 70%, 80%, 85%, 90%, 91%, 92%, 93% 94% 95%, 96%, 97%, 98%, 99% or 100% identical to a sequence selected from any one of SEQ ID NOs: 14-28.

In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 14. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 15. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 16. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 17. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 18. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 19. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 20. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 21. In some embodiments, the second constant region from an IgG heavy chain comprises an amino acid sequence that is at least 70%, 80%, 85%, 90%, 91%, 92%, 930, 94% 95%, 96%, 97%, 98%, 99% or 100% identical to a sequence of SEQ ID NO: 22. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 23. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 24. In some embodiments, the second constant region from an IgG heavy chain comprises an amino acid sequence that is at least 70%/o, 80%, 85%, 90%, 91%, 92%, 93%, 94% 95%, 96%, 97%, 98%, 99% or 100% identical to a sequence of SEQ ID NO: 25. In some embodiments, the second constant region from an IgG heavy chain comprises an amino acid sequence that is at least 70%, 80%, 85%, 90%, 91%, 92%, 93%, 94% 95%, 96% 97%, 98%, 99% or 100% identical to a sequence of SEQ ID NO: 26. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 27. In some embodiments, the second constant region from an IgG heavy chain comprises an amino acid sequence that is at least 70%, 80%, 85%, 90%, 91%, 92%, 93%, 94% 95%, 96%, 97%, 98%, 99% or 100% identical to a sequence of SEQ ID NO: 28.

In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 46, 48, 55, 57, 58, 59, 60, 61, 84, 86, 88, 89, 90, and 91.

In some embodiments. the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 46. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 48. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 55. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 57. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 58. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 59. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 60. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 61. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 84. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 86. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 88. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 89. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 90. In some embodiments, the first polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 91.

In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence selected from any one of SEQ ID NOs: 49, 51, 62, 63, 85, and 87.

In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 49. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 51. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 62. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 63. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 85. In some embodiments, the second polypeptide comprises an amino acid sequence that is at least 70%, 80%, 85%, 90%, 91%, 92%, 93%, 94% 95%, 96%, 97%, 98%, 99% or 100% identical to a sequence of SEQ ID NO: 87.

In some embodiments, a heteromultimer complex binds to one or more of ActRIIA or ActRIIB ligands selected from the group consisting of activin A, activin B, GDF11, GDF8, GDF3, BMP5, BMP6, and BMP10. In some embodiments, a single-arm ActRIIB heteromultimer binds to activin B and GDF11. In some embodiments, a single-arm ActRIIB heteromultimer binds to GDF8 and activin A. In some embodiments, a single-arm ActRIIA heteromultimer binds to activin A over activin B and GDF11. In some embodiments, a single-arm ActRIIA heteromultimer binds to GDF8.

### 3. Linkers

The disclosure provides for single-arm ActRIIA heteromultimers or single-arm ActRIIB heteromultimers, and in these embodiments, the ActRIIA or ActRIIB polypeptide and a first member of an interaction pair (e.g., a constant region from an IgG heavy chain) may be connected by means of a linker. In some embodiments, a single-arm ActRIIA heteromultimer comprises a linker domain positioned between the ActRIIA polypeptide and the first member of an interaction pair. In some embodiments, a single-arm ActRIIB heteromultimer comprises a linker domain positioned between the ActRIIB polypeptide and the first member of an interaction pair. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 29), or repetitions thereof (GGGGS)n, where n≥2 (SEQ ID NO: 30). In particular embodiments n≥3, or n=3-10. In preferred embodiments, n≥4, or n=4-10. In some embodiments, n is not greater than 4 in a (GGGGS)n linker (SEQ ID NO: 29). In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence (SEQ ID NO: 29) also comprises an N-terminal threonine. In some embodiments, the linker is any one of the following:

(SEQ ID NO: 31)
GGG
(SEQ ID NO: 32)
GGGG
(SEQ ID NO: 33)
GGGGSGGGGS
(SEQ ID NO: 34)
SGGG
(SEQ ID NO: 35)
SGGGG
(SEQ ID NO: 36)
TGGG
(SEQ ID NO: 37)
TGGGG
(SEQ ID NO: 38)
TGGGGSGGGGS
(SEQ ID NO: 39)
TGGGGSGGGGSGGGGS
(SEQ ID NO: 40)
TGGGGSGGGGSGGGGSGGGGS
(SEQ ID NO: 41)
TGGGGSGGGGSGGGGSGGGGSGGGGS
(SEQ ID NO: 42)
TGGGGSGGGGSGGGGSGGGGSGGGGSGGGGS
or
(SEQ ID NO: 43)
TGGGGSGGGGSGGGGSGGGGSGGGGSGGGGSGGGGS.

In some embodiments, the linker comprises the amino acid sequence of TGGGPKSCDK (SEQ ID NO: 44). In some embodiments, the linker is any one of SEQ ID NOs: 31-44) lacking the N-terminal threonine. In some embodiments, the linker does not comprise the amino acid sequence of SEQ ID NO: 42 or 43. In some embodiments, the linker comprises an amino acid sequence selected from any one of SEQ ID NOs: 29-44.

### 4. Nucleic Acids Encoding ActRIIA or ActRIIB Polypeptides

In certain embodiments, the present disclosure provides isolated and/or recombinant nucleic acids encoding ActRIIA or ActRIIB polypeptides (including fragments, functional variants, and fusion proteins thereof) disclosed herein. For example, SEQ ID NO: 12 encodes the naturally occurring human ActRIIA precursor polypeptide, while SEQ ID NO: 13 encodes the processed extracellular domain of ActRIIA. The subject nucleic acids may be single-stranded or double stranded. Such nucleic acids may be DNA or RNA molecules. These nucleic acids may be used, for example, in methods for making ActRIIA or ActRIIB single-arm heteromultimers of the present disclosure.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRIIA or ActRIIB polypeptides of the present disclosure are understood to include nucleic acids that are variants of any one of SEQ ID NOs: 7, 8, 12, and 13. In certain embodiments, nucleic acid encoding a first and/or second member of an interaction pair (e.g., a constant region from an IgG heavy chain) are understood to include nucleic acids that are variants of any one of SEQ ID NOs: 50. In some embodiments, nucleic acids encoding single-arm ActRIIA heteromultimer fusions or single-arm ActRIIB heteromultimer Fc fusions of the present disclosure are understood to include nucleic acids that are variants of any one of SEQ ID NOs: 47 and 56. In some embodiments, a single-arm ActRIIA heteromultimer fusion comprises a single-arm ActRIIA heterodimer Fc fusion, comprising the amino acid sequence of SEQ ID NO: 56. In some embodiments, a single-arm ActRIIB heteromultimer fusion comprises a single-arm ActRIIB heterodimer Fc fusion, comprising the amino acid sequence of SEQ ID NO: 47. Variant nucleotide sequences include sequences that differ by one or more nucleotide substitutions, additions, or deletions including allelic variants, and therefore, will include coding sequences that differ from the nucleotide sequence designated in any one of SEQ ID NOs: 7, 8, 12, 13, 47, 50, and 56. In certain embodiments, ActRIIA or ActRIIB polypeptides of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NOs: 7, 8, 12, 13. In certain embodiments, a first and/or second member of an interaction pair (e.g., a constant region from an IgG heavy chain) of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NOs: 50. In some embodiments, a single-arm ActRIIA heteromultimer fusion of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NO: 56. In some embodiments, a single-arm ActRIIB heteromultimer fusion of the present disclosure are encoded by isolated or recombinant nucleic acid sequences that are at least 80%, 85%, 90%, 91%, 92%, 93%, 94%, 95%, 96%, 97%, 98%, or 99% identical to SEQ ID NO: 47. One of ordinary skill in the art will appreciate that nucleic acid sequences that are at least 80%, 85%, 90%, 91%, 92%, 93%, 94%, 95%, 96%, 97%, 98%. or 99% identical to the sequences complementary to SEQ ID NOs: 7, 8, 12, 13, 47, 50, and 56 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under highly stringent conditions to the nucleotide sequence designated in SEQ ID NOs: 7, 8, 12, 13, 47, 50, and 56, the complement sequence of SEQ ID NOs: 7, 8, 12, 13, 47, 50, and 56, or fragments thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 47, 50, and 56 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, and operably linked to at least one regulatory sequence. Regulatory sequences are art-recognized and are selected to direct expression of the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer. Accordingly. the term regulatory sequence includes promoters, enhancers, and other expression control elements. Exemplary regulatory sequences are described in Goeddel: Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E coil.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer in CHO cells, such as a Pemv-Script vector (Stratagene, La Jolla, Calif.). pcDNA4 vectors (Invitrogen, Carlsbad, Calif.) and pCI-neo vectors (Promega, Madison, Wis.). As will be apparent, the subject gene constructs can be used to cause expression of the subject ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer in cells propagated in culture, e.g., to produce proteins, including fusion proteins or variant proteins, for purification.

This disclosure also pertains to a host cell transfected with a recombinant gene including a coding sequence for one or more of the subject ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer. The host cell may be any prokaryotic or eukaryotic cell. For example, an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer of the disclosure may be expressed in bacterial cells such as E. coli, insect cells (e.g., using a baculovirus expression system), yeast, or mammalian cells [e.g., a Chinese hamster ovary (CHO) cell line]. Other suitable host cells are known to those skilled in the art.

Accordingly, the present disclosure further pertains to methods of producing the subject ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer. For example, a host cell transfected with an expression vector encoding an ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer can be cultured under appropriate conditions to allow expression of the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer to occur. The polypeptide(s) may be secreted and isolated from a mixture of cells and medium containing the polypeptide(s). Alternatively, the ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer and affinity purification with an agent that binds to a domain fused to ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer (e.g., a protein A column may be used to purify any polypeptides disclosed herein). In some embodiments, ActRIIA or ActRIIB polypeptides, a first and/or second member of an interaction pair (e.g., a constant region from an IgG heavy chain), and/or a single-arm ActRIIA heteromultimer or single-arm ActRIIB heteromultimer comprise a domain which facilitates purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. A single-arm ActRIIA heteromultimer or single-arm, ActRIIB heteromultimer, for example, may be purified to a purity of >90%, >91%, >92%, >93%, >94%, >95%, >96%, >97%, >98%, or >99% as determined by size exclusion chromatography and >90%, >91%, >92%, >93%, >94%, >95%, >96%, >97%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRIIA or ActRIIB polypeptide, a first and/or second member of an interaction pair (e.g., a constant region from an IgG heavy chain), and/or a single-arm ActRIIA heteromultimer or single-arm ActRIIB heteromultimer, can allow purification of the expressed construct by affinity chromatography using a Ni²⁺ metal resin. The purification leader sequence can then be subsequently removed by treatment with enterokinase to provide the purified ActRIIA or ActRIIB polypeptide or protein complex. See, e.g., Hochuli et al. (1987) J. Chromatography 411:177; and Janknecht et al. (1991) PNAS USA 88:8972.

Techniques for making fusion genes are well known. Essentially. the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively. PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Screening Assays

In certain aspects, the present disclosure relates to the use of single-arm ActRIIA heteromultimers or single-arm ActRIIB heteromultimers to identify compounds (agents) which are agonists or antagonists of ActRIIA or ActRIIB. Compounds identified through this screening can be tested to assess their ability to treat renal diseases or conditions (e.g., Alport syndrome, focal segmental glomerulosclerosis (FSGS), polycystic kidney disease, chronic kidney disease). These compounds can be tested, for example, in animal models.

In certain aspects, the present disclosure relates to the use of the subject single-arm ActRIIA heteromultimers or single-arm ActRIIB heteromultimers to identify compounds (agents) which may be used to treat, prevent, or reduce the progression rate and/or severity of renal diseases or conditions, particularly treating, preventing or reducing the progression rate and/or severity of one or more renal-associated complications.

There are numerous approaches to screening for therapeutic agents for treating renal diseases or conditions by targeting signaling (e.g., Smad signaling) of one or more ActRIIA or ActRIIB ligands. In certain embodiments, high-throughput screening of compounds can be carried out to identify agents that perturb ActRIIA or ActRIIB ligand-mediated effects on a selected cell line. In certain embodiments, the assay is carried out to screen and identify compounds that specifically inhibit or reduce binding of an ActRIIA or ActRIIB ligand (e.g., activin A, activin B, GDF11, GDF8, GDF3, BMP5, BMP6, or BMP10) to its binding partner, such as ActRIIA or ActRIIB. Alternatively, the assay can be used to identify compounds that enhance binding of an ActRIIA or ActRIIB ligand to its binding partner such as ActRIIA or ActRIIB. In a further embodiment, the compounds can be identified by their ability to interact with ActRIIA or ActRIIB.

A variety of assay formats will suffice, and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin. fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between an ActRIIA or ActRIIB ligand (e.g., activin A, activin B. GDF11, GDF8, GDF3, BMP5, BMP6, or BMP10) to its binding partner, such as ActRIIA or ActRIIB.

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound's efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition comprising the ActRIIB polypeptide (e.g., a single-arm ActRIIB heteromultimer), and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between an ActRIIA or ActRIIB ligand and its binding protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between an ActRIIA or ActRIIB ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between an ActRIIA or ActRIIB ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between an ActRIIA or ActRIIB ligand and its binding protein [see, e.g., Vidal and Legrain, (1999) Nucleic Acids Res 27:919-29: Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with an ActRIIA or ActRIIB ligand. The interaction between the compound and the ActRIIA or ActRIIB ligand may be covalent or non-covalent. For example, such interaction can be identified at the protein level using in vitro biochemical methods, including photo-crosslinking, radiolabeled ligand binding, and affinity chromatography [see, e.g., Jakoby W B et al (1974) Methods in Enzymology 46:1]. In certain cases, the compounds may be screened in a mechanism-based assay, such as an assay to detect compounds which bind to an ActRIIA or ActRIIB ligand. This may include a solid-phase or fluid-phase binding event. Alternatively, the gene encoding an ActRIIA or ActRIIB ligand can be transfected with a reporter system (e.g., β-galactosidase, luciferase, or green fluorescent protein) into a cell and screened against the library preferably by high-throughput screening or with individual members of the library. Other mechanism-based binding assays may be used: for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 6. Therapeutic Uses

In part, the present disclosure relates to methods of treating renal diseases or conditions (e.g., Alport syndrome, focal segmental glomerulosclerosis (FSGS), polycystic kidney diseases, chronic kidney disease), comprising administering to a patient in need thereof an effective amount of a single-arm ActRIIA heteromultimer or single-arm ActRIIB heteromultimer, or combinations of single-arm ActRIIA heteromultimers or single-arm ActRIIB heteromultimers of the present disclosure. In some embodiments, a single-arm ActRIIA heteromultimer or single-arm ActRIIB heteromultimer, or combinations of single-arm ActRIIA heteromultimers or single-arm ActRIIB heteromultimers of the present disclosure can be used to treat or prevent a disease or condition that is associated with abnormal activity of a ActRIIA or ActRIIB polypeptide, and/or an ActRIIA or ActRIIB ligand (e.g., Activin A. activin B, GDF11, GDF8, GDF3, BMP5, BMP6, and BMP10).

In certain embodiments, the present invention provides methods of treating an individual in need thereof through administering to the individual a therapeutically effective amount of a single-arm ActRIIA heteromultimer or single-arm ActRIIB heteromultimer, or combinations of single-arm ActRIIA heteromultimers or single-arm ActRIIB heteromultimers, as described herein, optionally in combination with one or more additional active agents and/or supportive therapies.

The terms “renal” and “kidney” are used interchangeably herein.

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering a single-arm ActRIIA heteromultimer or a single-arm ActRIIB heteromultimer in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The terms “patient”, “subject”, or “individual” are used interchangeably herein and refer to either a human or a non-human animal. These terms include mammals, such as humans, non-human primates. laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The term “baseline” as used herein refers to an initial measurement that can be compared to. In some instances, a baseline measurement can be a measurement made while a subject is administered only standard of care (SOC). In some instances, a baseline measurement can be made without a subject being administered SOC. A baseline measurement can also be a measurement made prior to administration of a single-arm ActRIIA heteromultimer or a single-arm ActRIIB heteromultimer of the present disclosure and/or SOC.

In certain aspects, the disclosure contemplates the use of a single-arm ActRIIA heteromultimer or a single-arm ActRIIB heteromultimer, in combination with one or more additional active agents or other supportive therapy for treating or preventing a disease or condition (e.g., Alport syndrome, focal segmental glomerulosclerosis (FSGS), polycystic kidney disease, chronic kidney disease). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more a single-arm ActRIIA heteromultimer or a single-arm ActRIIB heteromultimers of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of a single-arm ActRIIA heteromultimer or a single-arm ActRIIB heteromultimer of the present disclosure with the additional active agent or therapy and/or the desired effect.

In some embodiments, the disclosure contemplates methods of treating one or more complications of a renal disease or condition comprising administering to a subject in need thereof an effective amount of a single-arm ActRIIA heteromultimer or a single-arm ActRIIB heteromultimer. In some embodiments, the disclosure contemplates methods of preventing one or more complications of a renal disease or condition comprising administering to a subject in need thereof an effective amount of a single-arm ActRIIA heteromultimer or a single-arm ActRIIB heteromultimer. In some embodiments, the disclosure contemplates methods of reducing the progression rate of a renal disease or condition comprising administering to a subject in need thereof an effective amount of a single-arm ActRIIA heteromultimer or a single-arm ActRIIB heteromultimer. In some embodiments, the disclosure contemplates methods of reducing the progression rate of one or more complications of a renal disease or condition comprising administering to a subject in need thereof an effective amount of a single-arm ActRIIA heteromultimer or a single-arm ActRIIB heteromultimer. In some embodiments, the disclosure contemplates methods of reducing the severity of a renal disease or condition comprising administering to a subject in need thereof an effective amount of a single-arm ActRIIA heteromultimer or a single-arm ActRIIB heteromultimer. In some embodiments, the disclosure contemplates methods of reducing the severity of one or more complications of a renal disease or condition comprising administering to a subject in need thereof an effective amount of a single-arm ActRIIA heteromultimer or a single-arm ActRIIB heteromultimer. In some embodiments, a renal disease or condition is selected from the group consisting of Alport syndrome, focal segmental glomerulosclerosis (FSGS), polycystic kidney disease, and chronic kidney disease. In some embodiments, a renal disease or condition is Alport syndrome. In some embodiments, a renal disease or condition is focal segmental glomerulosclerosis (FSGS). In some embodiments, a renal disease or condition is polycystic kidney disease. In some embodiments, a renal disease or condition is chronic kidney disease. In some embodiments, a subject has a decline in kidney function. In some embodiments, methods of the present disclosure slow kidney function decline.

In certain aspects, the disclosure relates to methods of treating, preventing, or reducing the progression rate and/or severity of a renal disease or condition comprising administering to a subject in need thereof an effective amount of a single-arm ActRIIA heteromultimer or single-arm ActRIIB heteromultimer. In some embodiments, the method relates to treating a subject with Alport Syndrome. In some embodiments, the method relates to treating a subject with a confirmatory genetic diagnosed Alport Syndrome.

Alport syndrome, also known as hereditary nephritis, is a genetically heterogeneous disease that results from mutations in genes encoding alpha-3, alpha-4, and alpha-5 chains of type IV collagen. Type IV collagen alpha chains are normally located in various basement membranes throughout the body, including the kidneys. Abnormalities in these chains can result in defective basement membranes at these sites, which in turn lead to clinical features of Alport syndrome (e.g., progressive glomerular disease).

Transmission of Alport syndrome can be X-linked, autosomal recessive, or autosomal dominant. In some embodiments, a subject has X-linked Alport syndrome. In some embodiments, the disclosure relates to methods of treating a subject that has X-linked Alport syndrome. X-linked transmission accounts for the majority of affected patients and arises from mutations in the COL4Δ5 gene on the X chromosome. In some embodiments, a subject has genetic defects in the COL4Δ5 gene. In some embodiments, the disclosure relates to methods of treating a subject that has one or more genetic defects in the COL4Δ5 gene. Autosomal recessive variant accounts for approximately 15 percent of patients with Alport syndrome and arises from genetic defects in either the COL4Δ3 or COL4Δ4 genes. In some embodiments, a subject has autosomal recessive Alport syndrome. Autosomal dominant disease appears to account for between about 20 to about 30 percent of patients with Alport syndrome and arises from heterozygous mutations in the COL4Δ3 or COL4Δ4 genes. In some embodiments, a subject has autosomal dominant Alport syndrome. In some embodiments, a subject has heterozygous mutations in the COL4Δ3 gene. In some embodiments, a subject has heterozygous mutations in the COL4Δ4 gene. In some embodiments, a subject has genetic defects in the COL4Δ3 gene. In some embodiments, the disclosure relates to methods of treating a subject that has one or more genetic defects in the COL4Δ3 gene. In some embodiments, a subject has genetic defects in the COL4Δ4 gene. In some embodiments, the disclosure relates to methods of treating a subject that has one or more genetic defects in the COL4Δ4 gene. In some embodiments, a subject has genetic defects in the COL4Δ3 and COL4A genes. In some embodiments, the disclosure relates to methods of treating a subject that has one or more genetic defects in the COL4Δ3 and COL4Δ4 genes. Some families exhibit digenic inheritance due to transmission of mutations in two of the three genes (COL4Δ3. COL4A 4. COL4Δ5). In some embodiments, a subject has mutations in two of the three genes (COL4Δ3. COL4Δ4 COL4Δ5). In some embodiments, the disclosure relates to methods of treating a subject that has one or more genetic defects in the COL4Δ3. COL4Δ4. and/or COL4Δ5 genes.

The classical presentation of Alport syndrome is based upon clinical manifestations of affected males with X-linked disease. In some embodiments, a subject with X-linked disease has a glomerular disease that progresses to end-stage renal disease (ESRD). Clinical presentation and course in patients with autosomal recessive disease is similar to those with X-linked disease. Patients with autosomal dominant disease generally exhibit more gradual loss of renal function.

Initially, renal manifestation of Alport syndrome is typically asymptomatic persistent microscopic hematuria (e.g., presence of blood in the urine), which is usually present in early childhood in affected patients. Since screening urinalysis is seldom performed in routine pediatric primary care, microscopic hematuria may not be detected unless the patient is screened because of an affected family member or found as an incidental finding for another issue. Gross hematuria may be the initial presenting finding and often occurs after an upper respiratory infection. However, recurrent episodes of gross hematuria are not uncommon especially during childhood. In some embodiments, the disclosure relates to methods of treating a subject that has asymptomatic persistent microscopic hematuria. In some embodiments, the disclosure relates to methods of treating a subject that has gross hematuria. In some embodiments, the disclosure relates to methods of treating a subject that has recurring episodes of gross hematuria. In some embodiments, the disclosure relates to methods of reducing the severity, occurrence, and/or duration of asymptomatic persistent microscopic hematuria, gross hematuria, or persistent microscopic hematuria in a subject in need thereof (e.g., a subject with Alport syndrome).

Patients with Alport syndrome typically have normal C3 levels, which is a component of the complement pathway that plays an integral role in the body's immune defenses. Decreased C3 may be associated with acute glomerulonephritis, membranoproliferative glomerulonephritis, immune complex disease, active systemic lupus erythematosus, septic shock, and end-stage liver disease, among other conditions. In early childhood, serum creatinine and blood pressure measurements are usually at normal levels as well. In some embodiments, the disclosure relates to methods of treating a subject with Alport syndrome that has normal levels of C3. In some embodiments, the disclosure relates to methods of treating a subject with Alport syndrome that has decreased levels of C3 compared to a baseline measurement. In some embodiments, the disclosure relates to methods of increasing C3 levels in a subject in need thereof (e.g., a subject with Alport syndrome).

Proteinuria, hypertension, and progressive renal insufficiency may develop in a subject with Alport syndrome. Proteinuria comprises a presence of excess proteins in urine. Albumin is a protein produced by the liver which makes up roughly 50%-60% of the proteins in the blood. Due to this, the concentration of albumin in the urine is one of the most sensitive indicators of any kidney disease, particularly for subjects with diabetes or hypertension, compared to a routine proteinuria examination. This measurement is often referred to as albuminuria. In some embodiments, the disclosure relates to methods of treating a subject that has proteinuria. In some embodiments, the disclosure relates to methods of treating a subject that has hypertension. In some embodiments, the disclosure relates to methods of treating a subject that has progressive renal insufficiency. In some embodiments, the disclosure relates to methods of reducing the severity, occurrence, and/or duration of one or more of proteinuria, hypertension, and progressive renal insufficiency in a subject in need thereof (e.g., a subject with Alport syndrome).

Subjects with Alport syndrome may develop end-stage renal disease (ESRD). ESRD usually occurs between the ages of 16 and 35 years in patients with X-linked or autosomal recessive Alport syndrome, among many other renal diseases and conditions. In some families, the course is more indolent with kidney failure being delayed until age 45 to 60, especially in those with autosomal dominant Alport syndrome. Females with X-linked Alport syndrome may have recurrent episodes of gross hematuria, proteinuria, and diffuse glomerular basement membrane (GBM) thickening are associated with more severe kidney dysfunction and ESRD at an earlier age. In some embodiments, the disclosure relates to methods of treating subjects with Alport syndrome that have ESRD. In some embodiments, the disclosure relates to methods of treating females with X-linked Alport syndrome. In some embodiments, the disclosure relates to methods of reducing severity, occurrence and/or duration of one or more of gross hematuria, proteinuria, and diffuse glomerular basement membrane (GBM) thickening are associated with more severe kidney dysfunction and ESRD in a subject in need thereof (e.g., a subject with Alport syndrome).

A diagnosis of Alport syndrome may be made by molecular genetic testing, or by skin or renal biopsy. Molecular genetic next generation analysis is a preferred method to make a diagnosis for patients with a positive family history for persistent hematuria and/or end-stage renal disease (ESRD) and for patients with chronic kidney disease (CKD). regardless of family history. Alport syndrome can be distinguished from other glomerular diseases by presence of a characteristic finding of lamination of the glomerular basement membrane (GBM) in samples from a renal biopsy, or abnormalities of type IV collagen by immunostaining, or by identification of one or more mutations in COL4Δ3, COL4Δ4, or COL4Δ5. Thin glomerular basement membranes in a subject with a COL4Δ3, COL4Δ4, or COL4AS mutation, with or without the manifestation of FSGS, is properly diagnosed as Alport syndrome. In some embodiments, the disclosure relates to methods of treating subjects with Alport syndrome that have a positive family history for persistent hematuria and/or end-stage renal disease (ESRD) and/or for patients with chronic kidney disease (CKD).

In certain aspects, the disclosure relates to methods of treating, preventing, or reducing the progression rate and/or severity of a renal disease or condition comprising administering to a subject in need thereof an effective amount of a single-arm ActRIIA heteromultimer or single-arm ActRIIB heteromultimer to subjects that have focal segmental glomerulosclerosis (FSGS). In some embodiments, the subject has primary FSGS. In some embodiments, the subject has genetic FSGS.

FSGS is a glomerular scarring disease characterized by an effacement of the podocyte foot on a kidney biopsy. When urine samples from subjects suffering FSGS are analyzed, a massive urine protein loss is typically observed, which can progress to a renal failure. FSGS is a common histopathologic lesion among adults with idiopathic nephrotic syndrome in the United States, accounting for about 35 percent of all cases. FSGS is also the most common primary glomerular disease identified in patients with end-stage renal disease (ESRD) in the United States. Prevalence of FSGS as a lesion associated with ESRD has risen. FSGS is characterized by the presence of sclerosis in parts (segmental) of at least one glomerulus (focal) of a kidney biopsy specimen, when examined by light microscopy (LM), immunofluorescence (IF), or electron microscopy (EM). In some embodiments, the disclosure relates to methods of reducing severity, occurrence and/or duration of urine protein loss in a subject in need thereof (e.g., a subject with FSGS). In some embodiments, the disclosure relates to methods of reducing severity, occurrence and/or duration of renal failure in a subject in need thereof (e.g., a subject with FSGS). In some embodiments, the disclosure relates to methods of reducing severity, occurrence and/or duration of end stage renal disease (ESRD) in a subject in need thereof (e.g., a subject with FSGS). In some embodiments, the disclosure relates to methods of reducing severity, occurrence and/or duration of sclerosis in a glomerulus of a kidney in a subject in need thereof (e.g., a subject with FSGS).

FSGS arises as a consequence of multiple pathways either individually or collectively resulting in injury to a podocyte, which is a cell in the Bowman's capsule in the kidneys that wraps around capillaries of the glomerulus. There are five known etiologies, and a suggested sixth etiology, associated with FSGS. Etiologies of FSGS comprise primary (e.g., idiopathic), secondary (e.g., adaptive), genetic, virus-associated, medication-associated, and APOL1 risk allele-associated. Primary or idiopathic FSGS is associated with a plasma factor with responsiveness to immunosuppressive therapy and a risk of recurrence after kidney transplant. In primary FSGS, a putative circulating factor that is toxic to a podocyte causes generalized podocyte dysfunction. Primary FSGS most often presents with the nephrotic system. Secondary (e.g., adaptive) FSGS is associated with excessive nephron workload due to increased body size, reduced nephron capacity, or single glomerular hyperfiltration associated with certain diseases. Secondary FSGS generally occurs as an adaptive phenomenon that results from a reduction in nephron mass, or can be considered as medicated-induced by direct toxicity from drugs (e.g., heroin, interferon, and pamidronate) or virus-induced by viral infections (e.g., HIV). Secondary FSGS often presents with non-nephrotic proteinuria, and/or with some degree of renal insufficiency. Secondary FSGS most commonly refers to FSGS that develops as an adaptive response to glomerular hypertrophy or hyperfiltration. Additional etiologies are recognized as drivers of FSGS, including high-penetrance genetic FSGS due to mutations in one of nearly 40 genes (genetic FSGS), virus-associated FSGS. and medication-associated FSGS. Emerging data support the identification of a sixth etiology: APOL1 risk allele-associated FSGS in individuals with sub-Saharan ancestry. Sometimes, secondary FSGS encompasses virus-associated FSGS and/or medication-associated FSGS. In some embodiments, the disclosure relates to methods of treating a subject with primary or idiopathic FSGS. In some embodiments, the disclosure relates to methods of treating a subject with secondary or adaptive FSGS. In some embodiments, the disclosure relates to methods of treating a subject with genetic FSGS. In some embodiments, the disclosure relates to methods of treating a subject with virus-associated FSGS. In some embodiments, the disclosure relates to methods of treating a subject with medication-associated FSGS. In some embodiments, the disclosure relates to methods of treating a subject with APOL1 risk allele-associated FSGS.

Primary FSGS comprises several prototypical characteristics. Primary FSGS is the most common form of FSGS in adolescents and young adults. and is commonly associated with nephrotic-range proteinuria (sometimes massive proteinuria, e.g., >10 g protein/day in the urine), reduced plasma albumin levels, and/or hyperlipidemia. In some embodiments, nephrotic-range proteinuria comprises proteinuria >3.5 g protein/day, and/or hypoalbuminemia <3.5 g albumin/dL urine (<35 g/L), and/or other manifestations of the nephrotic syndrome (e.g., edema, hyperlipidemia). In some embodiments, the disclosure relates to methods of reducing severity, occurrence and/or duration of one or more of nephrotic range proteinuria, reduced plasma albumin levels, or hyperlipidemia in a subject in need thereof (e.g., a subject with primary FSGS). In some embodiments, the disclosure relates to methods of reducing severity, occurrence and/or duration of proteinuria in a subject in need thereof (e.g., a subject with primary FSGS).

A subject with secondary or adaptive FSGS typically presents with slowly increasing proteinuria and renal insufficiency over time. Proteinuria in subjects with secondary FSGS often presents in the non-nephrotic range (e.g., nephrotic range is typically a loss of 3 grams or more of protein in the urine per day, and/or presence of 2 grams of protein per gram of creatinine in the urine). Sometimes, proteinuria in subjects with secondary FSGS comprises serum albumin levels that are normal. A subject with secondary FSGS may have s a glomerular filtration rate (GFR) that is elevated, which is a measurement of the flow rate of filtered fluid through the kidney. In some embodiments, a subject with secondary FSGS and an increase in GFR may have one or more additional and/or associated conditions selected from the group consisting of congenital cyanotic heart disease, sickle cell anemia, obesity, androgen abuse, sleep apnea, and high-protein diet. In some embodiments, the disclosure relates to methods of treating a subject with secondary FSGS with a normal GFR. In some embodiments, the disclosure relates to methods of treating a subject with secondary FSGS that has a decreased GFR. In some embodiments, the disclosure relates to methods of reducing severity, occurrence and/or duration of proteinuria and/or renal insufficiency in a subject in need thereof (e.g., a subject with primary FSGS).

Viruses have been implicated in causing FSGS. HIV-1 may be associated with FSGS, particularly the collapsing glomerulopathy variant. Other viruses that have been implicated in causing FSGS include, but are not limited to, cytomegalovirus, parvovirus B19, and Epstein-Barr virus. Parasites have also been associated with FSGS, which include, but are not limited to, Plasmodium (malaria), Schistosoma mansoni, and filiariasis. In some embodiments, the disclosure relates to methods of treating a subject with FSGS associated with HIV-1. In some embodiments, the disclosure relates to methods of treating a subject with FSGS associated with one or more of HIV-1, cytomegalovirus, parvovirus B19, and Epstein-Barr virus. In some embodiments, the disclosure relates to methods of treating a subject with FSGS associated with parasites including, but not limited to, Plasmodium (malaria), Schistosoma mansoni, and filiariasis. In some embodiments, the disclosure relates to methods of treating subjects with FSGS associated with an infection including, but not limited to, HIV, cytomegalovirus, parvovirus B19, Epstein-Barr virus, pulmonary tuberculosis, leishmaniasis, and malaria.

In some embodiments, the disclosure relates to methods of treating subjects with FSGS associated with autoimmune disorders implicated in causing FSGS including, but not limited to Adult Still's disease, systemic lupus erythematosus, and mixed connective tissue disorder.

In some embodiments, the disclosure relates to methods of treating subjects with FSGS associated with malignancies implicated in causing FSGS including, but not limited to hemophagocytic lymphohistiocytosis, multiple myeloma. and acute monoblastic leukemia.

In some embodiments, the disclosure relates to methods of treating subjects with FSGS associated with acute glomerular ischemias implicated in causing FSGS including, but not limited to thrombotic microangiopathy, renal infarction, atheroembolism, and hydrophilic polymer embolism.

In some embodiments, the disclosure relates to methods of treating subjects with FSGS associated with genetic disorders implicated in causing FSGS including, but not limited to APOL1 high-risk alleles, sickle cell disease. mitochondrial disorders (coenzyme Q deficiency), acute myoclonus-renal failure syndrome, and Galloway-Mowat syndrome.

In some embodiments, the disclosure relates to methods of treating subjects with FSGS associated with post transplantation events implicated in causing FSGS including, but not limited to Arteriopathy/thrombotic microangiopathy, acute rejection, and viral infection (cytomegalovirus, Epstein-Barr virus, BK polyomavirus).

In some embodiments, the disclosure relates to methods of treating subjects with FSGS associated with certain medications. In some embodiments, IFN-α, -β, or -γ therapy has been associated with development of collapsing glomerulopathy. In some embodiments, the disclosure relates to methods of treating subjects with FSGS associated with one or more of podocyte injury, including MCD, FSGS, and particularly, collapsing FSGS (collapsing glomerulopathy) who have taken and/or are still taking bisphosphonates. In some embodiments, the disclosure relates to methods of treating subjects with FSGS who have been on and/or are currently on lithium therapy. In some embodiments, the disclosure relates to methods of treating subjects with FSGS who have taken and/or are still taking sirolimus. In some embodiments, the disclosure relates to methods of treating subjects with FSGS who have taken and/or are still taking anthracycline medications, including doxorubicin (Adriamcyin) and daunomycin. In some embodiments, the disclosure relates to methods of treating subjects with FSGS who have taken and/or are still taking medications implicated in causing FSGS including, but not limited to bisphosphonates, interferons (alpha, beta, or gamma), anabolic steroids, calcineurin inhibitors, and mammalian (mechanistic) target of rapamycin (mTOR) inhibitors.

Genetic FSGS takes two forms. In some embodiments, the disclosure relates to methods of treating subjects with genetic FSGS associated with one or more variants in susceptibility genes (i.e., some individuals with a particular variant will develop FSGS, and other individuals will not). In some embodiments, the disclosure relates to methods of treating subjects with FSGS associated with one or more susceptibility genes including, but not limited to APOL1 and PDSS1. In some embodiments, the disclosure relates to methods of treating subjects with genetic FSGS associated with one or more high-penetrance mutations that manifest either Mendelian inheritance (for nuclear genes) or maternal inheritance (for genes encoded by mitochondrial DNA). The number of genes associated with FSGS rises every year, in large part because of the dissemination of whole-exome sequencing. At least 38 genes have been identified in relation to genetic FSGS. In some embodiments, the disclosure relates to methods of treating subjects with FSGS associated with one or more genes involved in genetic FSGS comprising COL4Δ3, COL4Δ4, COL4Δ5, ITGB4, LMB2, NPHS, NPHS2, CD2AP, PTPRO, MYO1E, ACTN4, INF2, AHRGP24, AHRGDIA, MYH9, INF1, MT-TL1, MT-TL2, MT-TY, COQ2, COQ6, PDSS2, ADCK, WT1, NUP95, NUP203, XP05, NXF5, PAX2, LMX1B, SMARCAL1, NXF5, EYA1, WDR73, LMNA, PLCE1, TRPC6, KANK4, SCARB2, and TTC21B.

In some embodiments, a subject suspected of FSGS is administered a kidney biopsy. A kidney biopsy may be analyzed by light microscopy to determine one or more of glomerular size, histologic variant of FSGS, microcystic tubular changes, and tubular hypertrophy. Further, a kidney biopsy may be analyzed by immunofluorescence to rule out other primary glomerulopathies and/or by electron microscopy to determine one or more of an extent of podocyte foot process effacement, podocyte microvillus transformation, and tubuloreticular inclusions. A complete assessment of renal histology is important for establishing the parenchymal setting of segmental glomerulosclerosis, distinguishing FSGS associated with one of many other glomerular diseases from the clinical-pathologic syndrome of FSGS. In some embodiments. genetic testing is used to further analyze a subject for a genetic FSGS etiology.

Traditionally. FSGS was classified based upon the Columbia classification, which defined five morphologic variants of FSGS lesions based upon LM examination. This classification system was designed to rely solely on pathologic criteria and does not integrate these findings with clinical and/or genetic information. In general, morphologic characteristics seen on kidney biopsy cannot distinguish between genetic and nongenetic forms of FSGS. Exceptions include distinctive features associated with NPHS1 and actinin alpha 4 gene mutations and the disease-specific lesions of Fabry disease, Alport syndrome, and lecithin-cholesterol acyl transferase deficiency. Histologic variants of FSGS comprise FSGS not otherwise specified (NOS) (formerly called classic FSGS, which is the most common form): collapsing variant, tip variant; perihilar variant; and cellular variant. Although the appearance of a glomerulus on LM, by definition, differs among these forms, they all share ultrastructural findings of podocyte alterations. Tip lesions affect the portion of the glomerular tuft juxtaposed to the tubular pole, and a tip lesion abnormality includes one or more of adhesion to Bowman's capsule at the tip, hypercellularity, presence of foam cells, and/or sclerosis. A collapsing variant shows segmental or global mesangial consolidation and loss of endocapillary patency in association with extracapillary epithelial hypertrophy and/or proliferation. Perihilar and NOS variants are determined by whether the segmental sclerosis/segmental obliteration of capillary loops with matrix increase (with or without hyalinosis) involves the segment near the hilum or the specific segment cannot be determined, respectively. A cellular lesion is the most difficult lesion to identify reproducibly. A cellular lesion shows segmental endocapillary hypercellularity occluding lumens with or without foam cells and karyorrhexis.

In certain aspects, the disclosure relates to methods of treating. preventing, or reducing the progression rate and/or severity of a renal disease or condition comprising administering to a subject in need thereof an effective amount of a single-arm ActRIIA heteromultimer or single-arm ActRIIB heteromultimer, wherein the renal disease or condition is polycystic kidney disease (PKD).

Polycystic Kidney Disease occurs in two forms: autosomal recessive (ARPKD) and autosomal dominant (ADPKD). The two forms of the disease have distinct genetic basis, and two genes involved in ADPKD have been identified, and one gene involved in ARPKD has been identified. The manifestations of the two different types of disease are very similar, and both result from a hyperproliferation of tubule epithelial cells that ultimately results in destruction of tubular structure with cyst formation leading to chronic renal failure. In some embodiments, the disclosure relates to methods of treating subjects with autosomal recessive polycystic kidney disease (ARPKD). In some embodiments, the disclosure relates to methods of treating subjects with autosomal dominant polycystic kidney disease (ADPKD).

Autosomal dominant polycystic kidney disease (ADPKD) is a hereditary disorder of the kidneys characterized by markedly enlarged kidneys with extensive cyst formation throughout. These cysts progressively enlarge with age, as kidney function gradually declines. A diagnosis of ADPKD is based on family history and ultra sonographic evaluation. In as many as 25% of patients with ADPKD, no family history is identified. which may be related to subclinical disease or a new genetic mutation in about 5% of such cases. A defining feature of ADPKD is marked bilateral, renal enlargement. Patients with ADPKD typically progress to end-stage renal disease (ESRD) by the fifth or sixth decade of life. The rate of progression of ADPKD is related directly to kidney volume, and therapies aim to slow the decline in renal volume to delay progression. In some embodiments, the disclosure relates to methods of reducing severity, occurrence and/or duration of cysts on the kidney in a subject in need thereof. In some embodiments, the disclosure relates to methods of reducing severity, occurrence and/or duration of renal enlargement in a subject in need thereof. In some embodiments, the disclosure relates to methods of reducing severity, occurrence and/or duration of an increase in kidney volume (e.g., total kidney volume) in a subject in need thereof.

ADPKD can be attributed to an abnormality on chromosome 16 (PKD1 locus) or chromosome 4 (PKD2 locus). PKD1 mutations comprise about 78% of ADPKD cases, while PKD2 mutations comprise about 14% of cases. PKD1 patients tend to progress to ESRD at an earlier age than PKD2 patients. In some embodiments, the disclosure relates to methods of treating a subject with ADPKD that has a mutation in the PKD1 locus. In some embodiments, the disclosure relates to methods of treating a subject with ADPKD that has a mutation in the PKD2 locus.

The PKD1 and PKD2 genes encode the proteins polycystin-1 and polycystin-2, respectively. These polycystins are integral membrane proteins and are found in renal tubular epithelia. It is postulated that abnormalities in polycystin-1 impair cell-cell and cell-matrix interactions in the renal tubular epithelia. while abnormalities in polycystin-2 impair calcium signaling in the cells.

Resultant changes in renal pathophysiology due to PKD include, but are not limited to, hematuria (often gross), a concentrating defect (resulting in polyuria and increased thirst), mild proteinuria, nephrolithiasis (in about 25% of ADPKD patients), flank pain, and abdominal pain. Furthermore, cyst rupture, hemorrhage, and infection are common complications. Progressive renal decline often results in end-stage renal disease. Hypertension is the most prevalent initial clinical presentation, occurring in about 50% to about 70% of cases, and is the most common feature directly associated with the rate of decline to ESRD and cardiovascular complications. In some embodiments, the disclosure relates to methods of reducing severity, occurrence and/or duration of one or more of hematuria, a concentrating defect, proteinuria, nephrolithiasis, flank pain, and abdominal pain in a subject in need thereof. In some embodiments, the disclosure relates to methods of reducing severity, occurrence and/or duration of one or more of cyst rupture, hemorrhage, and infection in a subject in need thereof. In some embodiments, the disclosure relates to methods of reducing severity, occurrence and/or duration of end-stage renal disease (ESRD) in a subject in need thereof. In some embodiments, the disclosure relates to methods of reducing severity, occurrence and/or duration of hypertension in a subject in need thereof.

Multiple extra-renal manifestations are often present in a subject with polycystic kidney disease. Cerebral aneurysms occur in about 5% of young adults, and as many as 20% of patients over the age of 60. Risk of a cerebral aneurysm or subarachnoid hemorrhage is highest in subjects with a family history of the same. Extrarenal cysts are common in ADPKD. Hepatic cysts are often noted in these patients, and prevalence increases with age. As many as 94% of patients over the age of 35 have been reported to have hepatic cysts. Total cyst prevalence and volume is higher in women versus men. Hepatic cysts in ADPKD patients rarely cause liver dysfunction. Rarely, patients develop pain from an acute cyst infection or hemorrhage. In addition, between about 7% and about 36% of ADPKD patients develop pancreatic cysts, with a higher prevalence in ADPKD patients with PKD2 mutations. Cardiac valvular disease has been noted in 25% to 30% of ADPKD patients. Cardiovascular complications, particularly cardiac hypertrophy and coronary artery disease, are leading causes of death in patients with ADPKD. In some embodiments, the disclosure relates to methods of reducing severity, occurrence and/or duration of a cerebral aneurysm in a subject in need thereof. In some embodiments, the disclosure relates to methods of reducing severity, occurrence and/or duration of extrarenal cysts in a subject in need thereof. In some embodiments, the disclosure relates to methods of reducing severity, occurrence and/or duration of hepatic cysts in a subject in need thereof. In some embodiments, the disclosure relates to methods of reducing severity, occurrence and/or duration of pancreatic cysts in a subject in need thereof. In some embodiments, the disclosure relates to methods of reducing severity, occurrence and/or duration of cardiovascular complications (e.g., cardiac hypertrophy, coronary artery disease) in a subject. in need thereof

Autosomal recessive polycystic kidney disease (ARPKD) is a cause of significant renal and liver-related morbidity and mortality in children. A majority of subjects with ARPKD present in the neonatal period with enlarged echogenic kidneys. Renal disease is characterized by nephromegaly, hypertension, and varying degrees of renal dysfunction. More than 50% of affected individuals with ARPKD progress to end-stage renal disease (ESRD) within the first decade of life, and subjects with ARPKD whom progressed to ESRD may require kidney transplantation. In some embodiments, the disclosure relates to methods of reducing severity, occurrence and/or duration of one or more nephromegaly, hypertension, and renal dysfunction in a subject in need thereof. In some embodiments, the disclosure relates to methods of reducing severity, occurrence and/or duration of end stage renal disease in a subject in need thereof, preventing a need for kidney transplantation.

ARPKD can be attributed to mutations in the PKHD1 gene located on chromosome 6p21, which contains at least 66 exons and encodes fibrocystin (also referred to as polyductin), a large integral membrane protein. Although the function of fibrocystin is presently unknown, it is found in the cortical and medullary collecting ducts and the thick ascending limb of the kidney, and in the epithelial cells of the hepatic bile duct. In some embodiments, the disclosure relates to methods of treating a subject with ARPKD that is associated with one or more mutations in PKHD1.

Because of the diversity of PKHD1 mutations, it can be challenging to correlate genotype with phenotype in cases of ARPKD. Subjects with two truncation mutations may have more severe renal involvement and are possibly at risk for early neonatal death. Subjects who are homozygotes for a missense mutation, or who have a missense mutation paired with a truncating mutation, may also have a severe phenotype. Subjects who are heterozygotes with two missense mutations typically have milder disease. Subjects who survive the neonatal period most often have at least one missense mutation. In some embodiments, the present disclosure relates to methods of treating a subject with ARPKD comprising two truncation mutations. In some embodiments, the present disclosure relates to methods of treating a subject with ARPKD comprising one or more missense mutations.

Two primary organ systems affected in ARPKD are the kidney and hepatobiliary tract. Kidneys may increase in size and/or have microcysts (usually less than 2 mm in size), which radiate from the medulla to the cortex, and are visible as pinpoint dots on the capsular surface. Severity of renal disease is proportional to the percentage of nephrons affected by cysts. Larger renal cysts (up to 1 cm) and interstitial fibrosis develop, which contribute to the progressive deterioration of renal function seen in subjects who survive beyond the neonatal period. ARPKD is associated with biliary dysgenesis due to a developmental defect comprising varying degrees of dilatation of the intrahepatic bile ducts and hepatic fibrosis. In some embodiments, the disclosure relates to methods of reducing severity, occurrence and/or duration of an increase in kidney size and/or presence of cysts.

Clinical presentation of ARPKD varies based on the age of onset of symptoms and the is predominance of hepatic or renal involvement. ARPKD is often detected by routine antenatal ultrasonography in fetuses after 24 weeks of gestation. A presumptive diagnosis is based on the presence of characteristic findings of markedly enlarged echogenic kidneys with poor corticomedullary differentiation. Discrete cysts ranging in size from 5 to 7 mm in diameter may be detected: however, larger cysts are unusual, especially those >10 mm in diameter. Subjects with ARPKD are typically monitored for blood pressure changes, renal function, serum electrolyte concentrations, hydration status, nutritional status, and growth. In some embodiments, the disclosure relates to methods of treating a subject with ARPKD further comprising monitoring one or more of blood pressure, renal function, serum electrolyte concentration, hydration status, nutritional status, and growth.

During the neonatal period, infants can present with renal manifestations, which may or may not be accompanied by respiratory distress. An infant with ARPKD may present with bilateral markedly enlarged kidneys, which may impact pulmonary function or lead to difficulty in feeding due to renal compression of the stomach. An infant with ARPKD may present with renal function impairment reflected by increased serum/plasma concentrations of creatinine and blood urea nitrogen (BUN). Neonates with end-stage renal disease (ESRD) may require renal replacement therapy (RRT) for survival. An infant with ARPKD may present with one or more of hypertension and hyponatremia (due to the inability to dilute urine maximally). In some embodiments, the disclosure relates to methods of reducing severity, occurrence and/or duration of renal function impairment reflected by increased serum/plasma concentrations of creatinine and blood urea nitrogen (BUN) in a subject in need thereof. In some embodiments, the disclosure relates to methods of reducing severity, occurrence and/or duration of hypertension and/or hyponatremia in a subject in need thereof.

For patients who survive beyond the neonatal period, there can be improvement of renal function due to continued renal maturation. However, over time, progressive deterioration of renal function can develop, which may be rapid or slow and may result in ESRD. An adolescent subject with ARPKD may have one or more of progressive deterioration of renal function (usually beginning with signs of tubular dysfunction or injury, polyuria and/or polydipsia due to a reduced concentrating ability, a maximal urine osmolality below 500 mosmol/kg, metabolic acidosis due to decreased urinary acidification capacity. hypertension, recurrent episodes of urinary tract infections, urinary abnormalities (including, but not limited to, mild proteinuria, glycosuria, hypophosphaturia, and/or increased urinary excretion of magnesium), progressive renal impairment, and decreased kidney growth rate and/or kidney size. In some embodiments, the present disclosure relates to methods of reducing severity, occurrence and/or duration of one or more of progressive deterioration of renal function, progressive renal impairment, and decreased kidney growth rate and/or kidney size in a subject in need thereof.

Ultrasound findings of ARPKD are characterized by bilateral large echogenic kidneys with poor corticomedullary differentiation. In patients with only medullary involvement, standard-resolution ultrasonography may be normal; however, high-resolution ultrasonography is able to detect ductal dilations confined to the medulla. Macrocysts, typically seen in subjects with autosomal dominant disease, are not usually present during infancy in patients with ARPKD, but may appear in older children. As a result, in older subjects, it may be more challenging to differentiate ARPKD from autosomal dominant polycystic kidney disease (ADPKD) by ultrasound. In some embodiments, the present disclosure relates to methods of treating a subject with ARPKD or ADPKD, further comprising differentiation of disease by ultrasound.

In certain aspects, the disclosure relates to methods of treating. preventing, or reducing the progression rate and/or severity of a renal disease or condition comprising administering to a subject in need thereof an effective amount of a single-arm ActRIIA heteromultimer or single-arm ActRIIB heteromultimer to a subject that has chronic kidney disease (CKD).

Chronic kidney disease (CKD) is a condition in which the kidneys are damaged and cannot filter blood as well as healthy kidneys. A subject with CKD typically has excess fluid and waste from blood remaining in the body. In some embodiments, the disclosure provides methods of treating a subject with CKD. In some embodiments, the disclosure relates to treating a subject with CKD, wherein the subject also has one or more other health conditions selected from the group consisting of anemia or low number of red blood cells, increased occurrence of infections, low calcium levels, high potassium levels, and high phosphorus levels in the blood, loss of appetite or eating less, depression or lower quality of life.

CKD has varying levels of seriousness and typically gets worse over time, though treatment has been shown to slow progression. If left untreated, CKD can progress to kidney failure, end stage renal disease (ESRD), and/or early cardiovascular disease, potentially leading to dialysis or kidney transplant for survival. In some embodiments, the present disclosure relates to methods of reducing severity, occurrence and/or duration of kidney failure, end stage renal disease (ESRD), and/or early cardiovascular disease in a subject in need thereof.

Diagnosis of CKD is typically accomplished by blood tests to measure the estimated glomerular filtration rate (eGFR), and/or a urine test to measure albumin and/or overall protein in the urine. Typically, an increase in protein in the urine indicates CKD. Ultrasound or kidney biopsy may be performed to determine an underlying cause.

In some embodiments, CKD manifests initially without symptoms, and is usually detected on routine screening blood work by either an increase in serum creatinine, and/or protein in the urine. As kidney function of a subject with CKD decreases, blood pressure increases due to fluid overload and production of vasoactive hormones created by the kidney via the renin-angiotensin system, thereby increasing the risk of developing hypertension and heart failure. As urea accumulates in a subject with CKD, azotemia and ultimately uremia (symptoms ranging from lethargy to pericarditis and encephalopathy) may arise. Due to its high systemic concentration, urea is excreted in eccrine sweat at high concentrations and crystallizes on skin as the sweat evaporates (e.g., “uremic frost”). In a subject with CKD, potassium may accumulate in the blood (e.g., hyperkalemia with a range of symptoms including malaise and potentially fatal cardiac arrhythmias). Hyperkalemia usually does not develop in a subject with CKD until the glomerular filtration rate (GFR) falls to less than about 20 to about 25 ml/min/1.73 m², at which point the kidneys have decreased ability to excrete potassium. Hyperkalemia in CKD can be exacerbated by acidemia (which leads to extracellular shift of potassium) and from lack of insulin. A subject with CKD may have hyperphosphatemia, which can result from poor phosphate elimination in the kidney.

Hyperphosphatemia contributes to increased cardiovascular risk by causing vascular calcification. A subject with CKD may have hypocalcemia. A subject with CKD may have one or more changes in mineral and bone metabolism that may cause abnormalities of calcium, phosphorus (phosphate), parathyroid hormone, or vitamin D metabolism; abnormalities in bone turnover, mineralization, volume, linear growth, or strength (kidney osteodystrophy); and/or vascular or other soft-tissue calcification. A subject with CKD may have metabolic acidosis that may result from decreased capacity to generate enough ammonia from the cells of the proximal tubule. A subject with CKD may have anemia. In later stages of CKD, a subject may develop cachexia. leading to unintentional weight loss, muscle wasting, weakness and anorexia. Subjects with CKD are more likely than the general population to develop atherosclerosis with consequent cardiovascular disease. In some embodiments, the present disclosure relates to methods of reducing severity, occurrence and/or duration of one or more conditions or complications of CKD selected from the group consisting of blood pressure increase, hypertension and/or heart failure. azotemia, uremia, “uremic frost”, hyperkalemia, decreased ability of the kidney to excrete potassium, acidemia, hyperphosphatemia, vascular calcification, hypocalcemia. changes in mineral and bone metabolism (particularly changes that may cause abnormalities of calcium, phosphorus (phosphate), parathyroid hormone, or vitamin D metabolism), abnormalities in bone turnover, mineralization, volume, linear growth, or strength (kidney osteodystrophy), vascular or other soft-tissue calcification, metabolic acidosis, anemia, cachexia (particularly cachexia that may lead to unintentional weight loss, muscle wasting, weakness and anorexia), and atherosclerosis (which may lead to cardiovascular disease).

Common causes of CKD are diabetes mellitus, hypertension, and glomerulonephritis. About one of five adults with hypertension and one of three adults with diabetes have CKD. CKD may also be caused by one or more of vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely. pinworms infecting the kidney can cause obstructive nephropathy. In some embodiments, the present disclosure relates to methods of treating a subject with CKD caused by one or more of diabetes mellitus, hypertension, and glomerulonephritis, vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely, pinworms infecting the kidney can cause obstructive nephropathy.

In some embodiments, the disclosure relates to methods of monitoring a subject with a renal disease or condition (e.g., Alport syndrome, focal segmental glomerulosclerosis (FSGS), polycystic kidney disease, chronic kidney disease) for albuminuria and/or proteinuria. Elevated protein levels in urine is a hallmark of many renal diseases or conditions. Annual monitoring for albuminuria and proteinuria are initiated beginning at one year of age for at-risk children. Proteinuria comprises a presence of abnormal quantities of protein in the urine. The most sensitive marker of proteinuria is elevated urine albumin (e.g., albuminuria). Albumin typically circulates in the blood, and only a trace of albumin is found in urine of subjects without a renal disease or condition. Moderate albuminuria is typically called microalbuminuria, while severe albuminuria is typically called macroalbuminuria. An albumin level above the upper limit value is called severe albuminuria or macroalbuminuria. In some embodiments, the present disclosure provides methods of treating a subject with one or more of albuminuria, proteinuria, microalbuminuria, and macroalbuminuria. In some embodiments, the present disclosure relates to methods of reducing severity, occurrence and/or duration of one or more of albuminuria, proteinuria, microalbuminuria, and macroalbuminuria in a subject in need thereof.

Measurements of albumin can have different units depending on how such measurements were taken. In some embodiments, albumin in urine is measured as a mass of albumin per time period of urine collected (e.g., mg/24 hr). In some embodiments, albumin in urine is measured as a mass of albumin per volume of urine collected (e.g., mg/L). In some embodiments, albumin in urine is measured as a mass of albumin per mass of creatinine in the urine (e.g., μg/mg of creatinine, termed albumin-creatine ratio, or ACR).

In some embodiments. a subject is administered a urine test to determine presence of a kidney disease or condition (e.g., Alport syndrome. focal segmental glomerulosclerosis (FSGS), polycystic kidney disease, chronic kidney disease). In some embodiments, a urine test comprises collection of urine over a specific time period (e.g., 24 hours). Moderate albuminuria or microalbuminuria comprises a level of albumin detected in the urine from a 24-hour urine collection that is between about 30 and about 300 mg albumin/24 hours and/or a level of albumin detected in the urine from a one minute urine collection that is between about 20 and about 200 μg albumin/l minute. Severe albuminuria or macroalbuminuria comprises a level of albumin detected in the urine from a 24-hour urine collection that is above about 300 mg albumin/24 hours and/or a level of albumin detected in the urine from a 1 minute urine collection that is above about 200 μg albumin/l minute. In some embodiments, the disclosure relates to methods of treating a subject with moderate albuminuria or microalbuminuria comprising a level of albumin detected in the urine from a 24-hour urine collection that is between about 30 and about 300 mg albumin/24 hours. In some embodiments, the disclosure relates to methods of treating a subject with moderate albuminuria or microalbuminuria comprising a level of albumin detected in the urine from a one minute urine collection that is between about 20 and about 200 μg albumin/1 minute. In some embodiments, the disclosure relates to methods of treating a subject with severe albuminuria or macroalbuminuria comprising a level of albumin detected in the urine from a 24-hour urine collection that is above about 300 mg albumin/24 hours. In some embodiments, the disclosure relates to methods of treating a subject with severe albuminuria or macroalbuminuria comprising a level of albumin detected in the urine from a 1 minute urine collection that is above about 200 μg albumin/i minute.

In some embodiments, a urine test comprises a spot test using a single sample of urine. In some embodiments, a urine test comprises a dipstick test. In some embodiments, a urine dipstick test may provide an estimate of the level of albuminuria. In some embodiments, moderate albuminuria or microalbuminuria comprises a level of albumin detected in the urine from a spot sample that is between about 20 and about 200 mg albumin/L urine. In some embodiments, severe albuminuria or macroalbuminuria comprises a level of albumin detected in the urine from a spot sample that is above about 200 mg albumin/L urine. In some embodiments, the disclosure relates to methods of treating a subject with moderate albuminuria or microalbuminuria comprising a level of albumin detected in a urine from a spot sample that is between about 20 and about 200 mg albumin/L urine. In some embodiments, the disclosure relates to methods of treating a subject with severe albuminuria or macroalbuminuria comprising a level of albumin detected in the urine from a spot sample that is above about 200 mg albumin/L urine.

To compensate for variations in urine concentration in spot-check samples (versus a larger sample collection and/or a sample collection over time), comparing the amount of albumin in the sample against the urine concentration of creatinine is useful. This is called the albumin/creatinine ratio (ACR). In some embodiments, presence and/or severity of albuminuria is determined by a ratio of albumin to creatinine in the urine (e.g., albumin-creatinine ratio, ACR, sometimes referred to as urinary albumin-creatinine ratio, or uACR). ACR lower and upper limits can vary between men and women. ACR is measured as a unit of mass of albumin per a unit of mass of creatinine in the urine. In some embodiments, the disclosure provides methods of treating a subject with moderate albuminuria or microalbuminuria comprising an ACR of between about 30 and about 300 mg albumin/g of creatinine. In some embodiments, the disclosure provides methods of treating a subject with severe albuminuria or macroalbuminuria comprising an ACR of above about 300 mg albumin/g of creatinine. In some embodiments, a normal ACR is typically below 30 mg albumin/g creatinine. It is important to note that the units of measure for any albuminuria measurement can differ. For example, ACR may be measured as μg of albumin per mg of creatinine. ACR may also be measured as g of albumin/g creatinine. Units of mg albumin/g creatinine are interchangeable with units of μg albumin/mg creatinine. ACR is sometimes provided without units, if both albumin and creatinine are provided as measurements of mass.

ACR can be measured as mass of albumin per concentration of creatinine in the urine. In some embodiments, the disclosure provides methods of treating moderate albuminuria or microalbuminuria comprising an ACR of between about 2.5 and about 35 mg albumin/mmol of creatinine in a subject in need thereof. In some embodiments, the disclosure provides methods of treating severe albuminuria or macroalbuminuria comprising an ACR of above about 35 mg albumin/mmol of creatinine in a subject in need thereof.

Disease stages describing the extent of renal damage and loss of function in a subject are typically assigned to subjects with renal diseases or conditions. Albuminuria stages are typically measured in terms of an ACR. In some embodiments, the present disclosure relates to methods of treating a subject with Stage Δ1 albuminuria. Stage A 1 albuminuria comprises normal to moderately increased levels of albumin in the urine, with an ACR of less than 30 mg albumin/g creatinine (or less than 3 mg albumin/mmol creatinine). In some embodiments, the present disclosure relates to methods of treating a subject with Stage Δ2 albuminuria. Stage Δ2 comprises moderate albuminuria or microalbuminuria, with an ACR of between about 30 and about 300 mg albumin/g creatinine (or between about 3 and about 30 mg albumin/mmol creatinine). In some embodiments, the present disclosure relates to methods of treating a subject with Stage Δ3 albuminuria. Stage Δ3 comprises severe albuminuria or macroalbuminuria, with an ACR of greater than 300 mg albumin/g creatinine (or greater than 30 mg albumin/mmol creatinine). In some embodiments, administration of therapy to a subject with a renal disease or condition will delay or prevent development of end stage renal disease. In some embodiments, administration of therapy to a subject with a renal disease or condition will lower said subject's albuminuria stage. In some embodiments, the present disclosure relates to methods of reducing severity, occurrence and/or duration of Stage Δ1 albuminuria. In some embodiments, the present disclosure relates to methods of reducing severity, occurrence and/or duration of Stage Δ2 albuminuria. In some embodiments, the present disclosure relates to methods of reducing severity, occurrence and/or duration of Stage Δ3 albuminuria. In some embodiments, the present disclosure provides methods of treating a subject with Stage A 1 albuminuria that delay or prevent progression to Stage Δ2 albuminuria. In some embodiments, the present disclosure provides methods of treating a subject with Stage Δ2 albuminuria that delay or prevent progression to Stage Δ3 albuminuria. In some embodiments, the present disclosure provides methods of delaying and/or preventing worsening of albuminuria stage progression in a subject in need thereof. In some embodiments, the present disclosure provides an improvement in renal damage and/or a downgrade in albuminuria stage classification in a subject in need thereof. In some embodiments, the present disclosure provides methods of improving albuminuria classification in a subject by one or more stages.

In some embodiments, a subject has proteinuria in the nephrotic range. In some embodiments, proteinuria in the nephrotic range comprises between about 3 and about 3.5 g of protein in the urine per 24 hours per 1.73 m² body surface area. In some embodiments, a subject with nephrotic syndrome has proteinuria of greater than 3.5 g/24 hrs/1.73 m².

In some embodiments, the disclosure relates to methods of reducing an ACR in a subject with a renal disease or condition, comprising administering to a subject in need thereof an effective amount of a single-arm ActRIIA heteromultimer or single-arm ActRIIB heteromultimer. In some embodiments, the method relates to reducing the subject's ACR by between about 0.1 and about 2.5 mg albumin/g creatinine compared to a baseline measurement. In some embodiments, the method relates to reducing the subject's ACR by between about 2.5 and about 3.5 mg albumin/g creatinine compared to a baseline measurement. In some embodiments, the method relates to reducing the subject's ACR by between about 3.5 and about 5.0 mg albumin/g creatinine compared to a baseline measurement. In some embodiments, the method relates to reducing the subject's ACR by between about 5.0 and about 7.5 mg albumin/g creatinine compared to a baseline measurement. In some embodiments. the method relates to reducing the subject's ACR by between about 7.5 and about 10.0 mg albumin/g creatinine compared to a baseline measurement. In some embodiments, the method relates to reducing the subject's ACR by between about 10.0 and about 15.0 mg albumin/g creatinine compared to a baseline measurement. In some embodiments, the method relates to reducing the subject's ACR by between about 15.0 and about 20.0 mg albumin/g creatinine compared to a baseline measurement. In some embodiments, the method relates to reducing the subject's ACR by between about 20.0 and about 25.0 mg albumin/g creatinine compared to a baseline measurement. In some embodiments, the method relates to reducing the subject's ACR by between about 30.0 and about 35.0 mg albumin/g creatinine compared to a baseline measurement. In some embodiments, the method relates to reducing the subject's ACR by between about 40.0 and about 45.0 mg albumin/g creatinine compared to a baseline measurement. In some embodiments, the method relates to reducing the subject's ACR by between about 45.0 and about 50.0 mg albumin/g creatinine compared to a baseline measurement. In some embodiments, the method relates to reducing the subject's ACR by between about 50.0 and about 60.0 mg albumin/g creatinine compared to a baseline measurement. In some embodiments, the method relates to reducing the subject's ACR by between about 60.0 and about 70.0 mg albumin/g creatinine compared to a baseline measurement. In some embodiments, the method relates to reducing the subject's ACR by between about 70.0 and about 80.0 mg albumin/g creatinine compared to a baseline measurement. In some embodiments, the method relates to reducing the subject's ACR by between about 80.0 and about 90.0 mg albumin/g creatinine compared to a baseline measurement. In some embodiments, the method relates to reducing the subject's ACR by between about 90.0 and about 100.0 mg albumin/g creatinine compared to a baseline measurement.

In some embodiments, the disclosure relates to methods of reducing an ACR in a subject with a renal disease or condition, comprising administering to a subject in need thereof an effective amount of a single-arm ActRIIA heteromultimer or single-arm ActRIIB heteromultimer. In some embodiments, the method relates to reducing the subject's ACR by greater than or equal to 0.5 g albumin/g creatinine compared to a baseline measurement. In some embodiments, the method relates to reducing the subject's absolute ACR to less than 0.5 g albumin/g creatinine compared to a baseline measurement. In some embodiments, the method relates to reducing the subject's absolute ACR to less than 0.3 g albumin/g creatinine compared to a baseline measurement. In some embodiments, the method relates to reducing the subject's ACR by between about 0.1 and about 2.5 g albumin/g creatinine compared to a baseline measurement. In some embodiments, the method relates to reducing the subject's ACR by between about 0.3 and about 2.5 g albumin/g creatinine compared to a baseline measurement. In some embodiments, the method relates to reducing the subject's ACR by between about 0.5 and about 2.5 g albumin/g creatinine compared to a baseline measurement. In some embodiments, the method relates to reducing the subject's ACR by between about 0.5 and about 3.0 g albumin/g creatinine compared to a baseline measurement. In some embodiments, the method relates to reducing the subject's ACR by between about 2.5 and about 3.5 g albumin/g creatinine compared to a baseline measurement. In some embodiments, the method relates to reducing the subject's ACR by between about 3.5 and about 5.0 g albumin/g creatinine compared to a baseline measurement. In some embodiments, the method relates to reducing the subject's ACR by between about 5.0 and about 7.5 g albumin/g creatinine compared to a baseline measurement. In some embodiments, the method relates to reducing the subject's ACR by between about 7.5 and about 10.0 g albumin/g creatinine compared to a baseline measurement. In some embodiments, the method relates to reducing the subject's ACR by between about 10.0 and about 15.0 g albumin/g creatinine compared to a baseline measurement. In some embodiments, the method relates to reducing the subject's ACR by between about 15.0 and about 20.0 g albumin/g creatinine compared to a baseline measurement. In some embodiments, the method relates to reducing the subject's ACR by between about 20.0 and about 25.0 g albumin/g creatinine compared to a baseline measurement. In some embodiments, the method relates to reducing the subject's ACR by between about 30.0 and about 35.0 g albumin/g creatinine compared to a baseline measurement. In some embodiments, the method relates to reducing the subject's ACR by between about 40.0 and about 45.0 g albumin/g creatinine compared to a baseline measurement. In some embodiments, the method relates to reducing the subject's ACR by between about 45.0 and about 50.0 g albumin/g creatinine compared to a baseline measurement. In some embodiments, the method relates to reducing the subject's ACR by between about 50.0 and about 60.0 g albumin/g creatinine compared to a baseline measurement. In some embodiments, the method relates to reducing the subject's ACR by between about 60.0 and about 70.0 g albumin/g creatinine compared to a baseline measurement. In some embodiments, the method relates to reducing the subject's ACR by between about 70.0 and about 80.0 g albumin/g creatinine compared to a baseline measurement. In some embodiments, the method relates to reducing the subject's ACR by between about 80.0 and about 90.0 g albumin/g creatinine compared to a baseline measurement. In some embodiments, the method relates to reducing the subject's ACR by between about 90.0 and about 100.0 g albumin/g creatinine compared to a baseline measurement.

In some embodiments, the method relates to reducing the subject's ACR by at least 2.5% compared to a baseline measurement. In some embodiments, the method relates to reducing the subject's ACR by at least 5% compared to a baseline measurement (e.g., SOC). In some embodiments, the method relates to reducing the subject's ACR by at least 10% compared to a baseline measurement. In some embodiments, the method relates to reducing the subject's ACR by at least 15% compared to a baseline measurement. In some embodiments, the method relates to reducing the subject's ACR by at least 20% compared to a baseline measurement. In some embodiments, the method relates to reducing the subject's ACR by at least 25% compared to a baseline measurement. In some embodiments, the method relates to reducing the subject's ACR by at least 30% compared to a baseline measurement. In some embodiments, the method relates to reducing the subject's ACR by greater than or equal to 30% compared to a baseline measurement. In some embodiments, the method relates to reducing the subject's ACR by at least 40% compared to a baseline measurement. In some embodiments, the method relates to reducing the subject's ACR by greater than or equal to 30% compared to a baseline measurement. In some embodiments, the method relates to reducing the subject's ACR by at least 50% compared to a baseline measurement. In some embodiments, the method relates to reducing the subject's ACR by greater than or equal to 50% compared to a baseline measurement. In some embodiments, the method relates to reducing the subject's ACR by at least 60% compared to a baseline measurement. In some embodiments, the method relates to reducing the subject's ACR by at least 70% compared to a baseline measurement. In some embodiments, the method relates to reducing the subject's ACR by at least 80% compared to a baseline measurement. In some embodiments, the method relates to reducing the subject's ACR by at least 90% compared to a baseline measurement. In some embodiments, the method relates to reducing the subject's ACR by at least 95% compared to a baseline measurement. In some embodiments, the method relates to reducing the subject's ACR by at least 99% compared to a baseline measurement.

In some embodiments, total urine protein may be measured and compared against creatinine presence in the urine (e.g., UPCR). In some embodiments, UPCR is a measurement of proteinuria. In some embodiments, proteinuria comprises a urinary protein-creatinine ratio (UPCR) of greater than 0.2 mg/mg. In some embodiments, proteinuria comprises a urinary protein excretion of greater than 4 mg/m² per hour. In some embodiments, complete remission (CR) of a renal disease or condition is defined as a consistent UPCR measurement of less than 0.2 g protein/g creatinine. In some embodiments, a partial remission (PR) of a renal disease or condition is defined as having about a 50% reduction from baseline proteinuria and a consistent UPCR of less than about 2 g protein/g creatinine.

In some embodiments, the disclosure relates to methods of reducing an UPCR in a subject with a renal disease or condition, comprising administering to a subject in need thereof an effective amount of a single-arm ActRIIA heteromultimer or single-arm ActRIIB heteromultimer. In some embodiments, the method relates to reducing the subject's UPCR by between about 0.2 and about 1 mg urinary protein/mg creatinine. In some embodiments, the method relates to reducing the subject's UPCR by less than 0.5 mg urinary protein/mg creatinine. In some embodiments, the method relates to reducing the subject's UPCR by between about by between about 0.1 and about 100.0 mg urinary protein/mg creatinine. In some embodiments, the method relates to reducing the subject's UPCR by between about 0.1 and about 2.5 mg urinary protein/mg creatinine. In some embodiments, the method relates to reducing the subject's UPCR by between about 2.5 and about 3.5 mg urinary protein/mg creatinine. In some embodiments, the method relates to reducing the subject's UPCR by between about 3.5 and about 5.0 mg urinary protein/mg creatinine. In some embodiments, the method relates to reducing the subject's UPCR by between about 5.0 and about 7.5 mg urinary protein/mg creatinine. In some embodiments, the method relates to reducing the subject's UPCR by between about 7.5 and about 10.0 mg urinary protein/mg creatinine. In some embodiments, the method relates to reducing the subject's UPCR by between about 10.0 and about 15.0 mg urinary protein/mg creatinine. In some embodiments, the method relates to reducing the subject's UPCR by between about 15.0 and about 20.0 mg urinary protein/mg creatinine. In some embodiments, the method relates to reducing the subject's UPCR by between about 20.0 and about 25.0 mg urinary protein/mg creatinine. In some embodiments, the method relates to reducing the subject's UPCR by between about 30.0 and about 35.0 mg urinary protein/mg creatinine. In some embodiments, the method relates to reducing the subject's UPCR by between about 40.0 and about 45.0 mg urinary protein/mg creatinine. In some embodiments, the method relates to reducing the subject's UPCR by between about 45.0 and about 50.0 mg urinary protein/mg creatinine. In some embodiments, the method relates to reducing the subject's UPCR by between about 50.0 and about 60.0 mg urinary protein/mg creatinine. In some embodiments, the method relates to reducing the subject's UPCR by between about 60.0 and about 70.0 mg urinary protein/mg creatinine. In some embodiments, the method relates to reducing the subject's UPCR by between about 70.0 and about 80.0 mg urinary protein/mg creatinine. In some embodiments, the method relates to reducing the subject's UPCR by between about 80.0 and about 90.0 mg urinary protein/mg creatinine. In some embodiments, the method relates to reducing the subject's UPCR by between about 90.0 and about 100.0 mg urinary protein/mg creatinine.

In some embodiments, the disclosure relates to methods of reducing an UPCR in a subject with a renal disease or condition, comprising administering to a subject in need thereof an effective amount of a single-arm ActRIIA heteromultimer or single-arm ActRIIB heteromultimer. In some embodiments, the method relates to reducing the subject's UPCR by between about 0.2 and about 1 g urinary protein/g creatinine. In some embodiments, the method relates to reducing the subject's UPCR by less than 0.5 g urinary protein/g creatinine. In some embodiments, the method relates to reducing the subject's UPCR by greater than or equal to 0.5 g urinary protein/g creatinine compared to a baseline measurement. In some embodiments, the method relates to reducing the subject's absolute UPCR to less than 0.5 g urinary protein/g creatinine compared to a baseline measurement. In some embodiments, the method relates to reducing the subject's absolute UPCR to less than 0.3 g urinary protein/g creatinine compared to a baseline measurement. In some embodiments, the method relates to reducing the subject's UPCR by between about by between about 0.1 and about 100.0 g urinary protein/g creatinine. In some embodiments, the method relates to reducing the subject's UPCR by between about 0.1 and about 2.5 g urinary protein/g creatinine. In some embodiments, the method relates to reducing the subject's UPCR by between about 2.5 and about 3.5 g urinary protein/g creatinine. In some embodiments, the method relates to reducing the subject's UPCR by between about 3.5 and about 5.0 g urinary protein/g creatinine. In some embodiments, the method relates to reducing the subject's UPCR by between about 5.0 and about 7.5 g urinary protein/g creatinine. In some embodiments, the method relates to reducing the subject's UPCR by between about 7.5 and about 10.0 g urinary protein/g creatinine. In some embodiments, the method relates to reducing the subject's UPCR by between about 10.0 and about 15.0 g urinary protein/g creatinine. In some embodiments, the method relates to reducing the subject's UPCR by between about 15.0 and about 20.0 g urinary protein/g creatinine. In some embodiments, the method relates to reducing the subject's UPCR by between about 20.0 and about 25.0 g urinary protein/g creatinine. In some embodiments, the method relates to reducing the subject's UPCR by between about 30.0 and about 35.0 g urinary protein/g creatinine. In some embodiments, the method relates to reducing the subject's UPCR by between about 40.0 and about 45.0 g urinary protein/g creatinine. In some embodiments, the method relates to reducing the subject's UPCR by between about 45.0 and about 50.0 g urinary protein/g creatinine. In some embodiments, the method relates to reducing the subject's UPCR by between about 50.0 and about 60.0 g urinary protein/g creatinine. In some embodiments, the method relates to reducing the subject's UPCR by between about 60.0 and about 70.0 g urinary protein/g creatinine. In some embodiments, the method relates to reducing the subject's UPCR by between about 70.0 and about 80.0 g urinary protein/g creatinine. In some embodiments, the method relates to reducing the subject's UPCR by between about 80.0 and about 90.0 g urinary protein/g creatinine. In some embodiments, the method relates to reducing the subject's UPCR by between about 90.0 and about 100.0 g urinary protein/g creatinine.

In some embodiments, administration of therapy decreases urinary protein excretion. In some embodiments, the method relates to reducing the subject's UPCR by at least 2.5% compared to a baseline measurement. In some embodiments, the method relates to reducing the subject's UPCR by at least 5% compared to a baseline measurement. In some embodiments, the method relates to reducing the subject's UPCR by at least 10% compared to a baseline measurement. In some embodiments, the method relates to reducing the subject's UPCR by at least 15% compared to a baseline measurement. In some embodiments, the method relates to reducing the subject's UPCR by at least 20% compared to a baseline measurement. In some embodiments, the method relates to reducing the subject's UPCR by at least 25% compared to a baseline measurement. In some embodiments, the method relates to reducing the subject's UPCR by at least 30% compared to a baseline measurement. In some embodiments, the method relates to reducing the subject's UPCR by greater than or equal to 30% compared to a baseline measurement. In some embodiments, the method relates to reducing the subject's UPCR by at least 40% compared to a baseline measurement. In some embodiments, the method relates to reducing the subject's UPCR by greater than or equal to 40% compared to a baseline measurement. In some embodiments, the method relates to reducing the subject's UPCR by at least 50% compared to a baseline measurement. In some embodiments, the method relates to reducing the subject's UPCR by greater than or equal to 50% compared to a baseline measurement. In some embodiments, the method relates to reducing the subject's UPCR by at least 60% compared to a baseline measurement. In some embodiments, the method relates to reducing the subject's UPCR by at least 70% compared to a baseline measurement. In some embodiments, the method relates to reducing the subject's UPCR by at least 80% compared to a baseline measurement. In some embodiments, the method relates to reducing the subject's UPCR by at least 90% compared to a baseline measurement. In some embodiments, the method relates to reducing the subject's UPCR by at least 95% compared to a baseline measurement. In some embodiments, the method relates to reducing the subject's UPCR by at least 99% compared to a baseline measurement.

A subject may be administered a blood test to determine presence of a kidney disease or condition (e.g., Alport syndrome, focal segmental glomerulosclerosis (FSGS), polycystic kidney disease, chronic kidney disease), by determining how well the kidney is filtering the blood. Typically, a glomerular filtration rate (GFR) is determined, which measures the flow rate of filtered fluid (e.g., blood) through the kidney into the Bowman's capsule. GFR is equal to the clearance rate of when any solute is freely filtered and is neither reabsorbed nor secreted by the kidneys. GFR is therefore a measurement of the quantity of the substance in the urine that originated from a calculable volume of blood, and is typically recorded in units of volume per time, e.g., milliliters per minute (mL/min). A normal range of GFR, adjusted for body surface area, is between about 100 and about 130 mL/min/1.73 m² in men, with an average GFR of 125 mL/min/1.73 m² in men. A normal range of GFR, adjusted for body surface area, is between about 90 and about 120 mL/min/1.73 m² in women younger than age 40. GFR measured by inulin clearance in children under 2 years old is about 110 mL/min/1.73 m², which progressively decreases. After age 40. GFR decreases progressively with age, by between about 0.4 and about 1.2 mL/min per year. GFR may also be calculated by comparative measurements of substances in the blood and urine, estimated using a blood test result (e.g., eGFR). In some embodiments, eGFR is measured using serum creatinine, age, ethnicity, and gender variables. In some embodiments, eGFR is measured using one or more of Cockcroft-Gault formula, Modification of Diet in Renal Disease (MDRD) formula, CKD-EPI formula, Mayo quadratic formula, and Schwartz formula.

A glomerular filtration rate (GFR)≥60 ml/min/1.73 m2 is considered normal in a subject without chronic kidney disease if there is no kidney damage present, which comprises signs of damage seen in blood, urine, or imaging studies which includes lab albumin/creatinine ratio (ACR)≥30. Subjects with a GFR <60 ml/min/1.73 m2 for at least 3 months are diagnosed as having chronic kidney disease.

In general, protein in the urine is regarded as an independent marker for decline of kidney function and cardiovascular disease, and the stages of chronic kidney disease (often used for renal diseases and/or conditions in general) is determined by measuring a subject's GFR. In some embodiments, the present disclosure provides methods of treating stage 1 CKD. Stage 1 CKD comprises normal kidney function, kidney damage with normal or relatively high GFR (e.g., ≥90 ml/min/1.73 m²), and lower creatinine levels. Kidney damage may be defined as pathological abnormalities or markers of damage. including abnormalities in blood or urine tests or imaging studies. In some embodiments, the present disclosure provides methods of treating stage 2 CKD. Stage 2 CKD comprises mild reduction in kidney function and GFR (e.g., between about 60 and about 89 ml/min/1.73 m²) with kidney damage. In some embodiments. the present disclosure provides methods of treating stage 3 CKD. Stage 3 CKD comprises mild to moderate reduction in kidney function and GFR (e.g., between about 30 and about 59 ml/min/1.73 m²). Stage 3 CKD may be split into stages 3a (e.g., mild to moderate reduction in kidney function and GFR between about 45 and about 59 ml/min/1.73 m² and 3b (e.g., moderate to severe reduction in kidney function and GFR between about 30 and about 44 ml/min/1.73 m². In some embodiments, the present disclosure provides methods of treating stage 3a CKD. In some embodiments, the present disclosure provides methods of treating stage 3b CKD. In some embodiments, the present disclosure provides methods of treating stage 4 CKD. Stage 4 CKD comprises severe reduction in kidney function and GFR (e.g., between about 15 and about 29 ml/min/1.73 m²). In some embodiments, the present disclosure provides methods of treating stage 5 CKD. Stage 5 CKD comprises established kidney failure (e.g., GFR about <15 ml/min/1.73 m²), permanent kidney replacement therapy, end-stage renal disease. (ESRD), and high creatinine levels. In some embodiments, the present disclosure relates to methods of reducing severity, occurrence and/or duration of stage 1 CKD. In some embodiments, the present disclosure relates to methods of reducing severity, occurrence and/or duration of stage 2 CKD. In some embodiments, the present disclosure relates to methods of reducing severity, occurrence and/or duration of stage 3 CKD. In some embodiments, the present disclosure relates to methods of reducing severity, occurrence and/or duration of stage 3a CKD. In some embodiments, the present disclosure relates to methods of reducing severity, occurrence and/or duration of stage 3b CKD. In some embodiments, the present disclosure relates to methods of reducing severity, occurrence and/or duration of stage 4 CKD. In some embodiments, the present disclosure relates to methods of reducing severity, occurrence and/or duration of stage 5 CKD. In some embodiments, the present disclosure provides methods of treating a subject with Stage 1 CKD that delay or prevent progression to Stage 2 CKD. In some embodiments, the present disclosure provides methods of treating a subject with Stage 2 CKD that delay or prevent progression to Stage 3 CKD. In some embodiments, the present disclosure provides methods of treating a subject with Stage 2 CKD that delay or prevent progression to Stage 3a CKD. In some embodiments, the present disclosure provides methods of treating a subject with Stage 2 CKD that delay or prevent progression to Stage 3b CKD. In some embodiments, the present disclosure provides methods of treating a subject with Stage 3a CKD that delay or prevent progression to Stage 3b CKD. In some embodiments, the present disclosure provides methods of treating a subject with Stage 3 CKD that delay or prevent progression to Stage 4 CKD. In some embodiments, the present disclosure provides methods of treating a subject with Stage 3a CKD that delay or prevent progression to Stage 4 CKD. In some embodiments, the present disclosure provides methods of treating a subject with Stage 3b CKD that delay or prevent progression to Stage 4 CKD. In some embodiments, the present disclosure provides methods of treating a subject with Stage 4 CKD that delay or prevent progression to Stage 5 CKD. In some embodiments, the present disclosure provides methods of delaying and/or preventing worsening of CKD stage progression in a subject in need thereof. In some embodiments, the present disclosure provides an improvement in renal damage and/or a downgrade in CKD stage classification in a subject in need thereof. In some embodiments, the present disclosure provides methods of improving CKD classification in a subject by one or more stages.

In certain aspects, the disclosure relates to methods of treating, preventing, or reducing the progression rate and/or severity of a renal disease or condition (e.g., Alport syndrome, focal segmental glomerulosclerosis (FSGS), polycystic kidney diseases, chronic kidney disease) comprising administering to a subject in need thereof an effective amount of a single-arm ActRIIA heteromultimer or single-arm ActRIIB heteromultimer. In some embodiments, the subject has stage 1 CKD. In some embodiments, the subject has stage 2 CKD. In some embodiments, the subject has stage 3 CKD. In some embodiments, the subject has stage 3a CKD. In some embodiments, the subject has stage 3b CKD. In some embodiments, the subject has stage 4 CKD. In some embodiments, the subject has stage 5 CKD.

In some embodiments, the disclosure relates to methods of increasing GFR and/or eGFR in a subject with a renal disease or condition, comprising administering to a subject in need thereof an effective amount of a single-arm ActRIIA heteromultimer or single-arm ActRIIB heteromultimer. In some embodiments, the method relates to increasing the subject's GFR and/or eGFR by at least 2.5% compared to a baseline measurement. In some embodiments, the method relates to increasing the subject's GFR and/or eGFR by at least 5% compared to a baseline measurement. In some embodiments, the method relates to increasing the subject's GFR and/or eGFR by at least 10% compared to a baseline measurement. In some embodiments, the method relates to increasing the subject's GFR and/or eGFR by at least 15% compared to a baseline measurement. In some embodiments. the method relates to increasing the subject's GFR and/or eGFR by at least 20% compared to a baseline measurement. In some embodiments, the method relates to increasing the subject's GFR and/or eGFR by at least 25% compared to a baseline measurement. In some embodiments, the method relates to increasing the subject's GFR and/or eGFR by at least 30% compared to a baseline measurement. In some embodiments, the method relates to increasing the subject's GFR and/or eGFR by greater than or equal to 30% compared to a baseline measurement. In some embodiments, the method relates to increasing the subject's GFR and/or eGFR by at least 40% compared to a baseline measurement. In some embodiments, the method relates to increasing the subject's GFR and/or eGFR by greater than or equal to 40% compared to a baseline measurement. In some embodiments, the method relates to increasing the subject's GFR and/or eGFR by at least 50% compared to a baseline measurement. In some embodiments, the method relates to increasing the subject's GFR and/or eGFR by at least 60% compared to a baseline measurement. In some embodiments, the method relates to increasing the subject's GFR and/or eGFR by at least 70% compared to a baseline measurement. In some embodiments, the method relates to increasing the subject's GFR and/or eGFR by at least 80% compared to a baseline measurement. In some embodiments, the method relates to increasing the subject's GFR and/or eGFR by at least 90% compared to a baseline measurement. In some embodiments, the method relates to increasing the subject's GFR and/or eGFR by at least 95% compared to a baseline measurement. In some embodiments, the method relates to increasing the subject's GFR and/or eGFR by at least 99% compared to a baseline measurement.

In some embodiments, the method relates to increasing the subject's eGFR and/or GFR and/or eGFR and/or GFR by about 1 mL/min/1.73 m² compared to a baseline measurement. In some embodiments, the method relates to increasing the subject's eGFR and/or GFR by about 3 mL/min/1.73 m² compared to a baseline measurement. In some embodiments, the method relates to increasing the subject's eGFR and/or GFR by about 5 mL/min/1.73 m² compared to a baseline measurement. In some embodiments, the method relates to increasing the subject's eGFR and/or GFR by about 7 mL/min/1.73 m² compared to a baseline measurement. In some embodiments, the method relates to increasing the subject's eGFR and/or GFR by about 9 mL/min/1.73 m² compared to a baseline measurement. In some embodiments, the method relates to increasing the subject's eGFR and/or GFR by about 10 mL/min/1.73 m² compared to a baseline measurement. In some embodiments, the method relates to increasing the subject's eGFR and/or GFR by about 15 mL/min/1.73 m² compared to a baseline measurement. In some embodiments, the method relates to increasing the subject's eGFR and/or GFR by about 20 mL/min/1.73 m² compared to a baseline measurement. In some embodiments, the method relates to increasing the subject's eGFR and/or GFR by about 25 mL/min/1.73 m² compared to a baseline measurement. In some embodiments, the method relates to increasing the subject's eGFR and/or GFR by about 30 mL/min/1.73 m² compared to a baseline measurement. In some embodiments, the method relates to increasing the subject's eGFR and/or GFR by about 35 mL/min/1.73 m² compared to a baseline measurement. In some embodiments, the method relates to increasing the subject's eGFR and/or GFR by about 40 mL/min/1.73 m² compared to a baseline measurement. In some embodiments, the method relates to increasing the subject's eGFR and/or GFR by about 45 mL/min/1.73 m² compared to a baseline measurement. In some embodiments, the method relates to increasing the subject's eGFR and/or GFR by about 50 mL/min/1.73 m² compared to a baseline measurement. In some embodiments, the method relates to increasing the subject's eGFR and/or GFR by about 55 mL/min/1.73 m² compared to a baseline measurement. In some embodiments. the method relates to increasing the subject's eGFR and/or GFR by about 60 mL/min/1.73 m² compared to a baseline measurement. In some embodiments, the method relates to increasing the subject's eGFR and/or GFR by about 65 mL/min/1.73 m² compared to a baseline measurement. In some embodiments, the method relates to increasing the subject's eGFR and/or GFR by about 70 mL/min/1.73 m² compared to a baseline measurement. In some embodiments, the method relates to increasing the subject's eGFR and/or GFR by about 75 mL/min/1.73 m² compared to a baseline measurement. In some embodiments. the method relates to increasing the subject's eGFR and/or GFR by about 80 mL/min/1.73 m² compared to a baseline measurement. In some embodiments, the method relates to increasing the subject's eGFR and/or GFR by about 85 mL/min/1.73 m² compared to a baseline measurement. In some embodiments, the method relates to increasing the subject's eGFR and/or GFR by about 90 mL/min/1.73 m² compared to a baseline measurement. In some embodiments, the method relates to increasing the subject's eGFR and/or GFR by about 95 mL/min/1.73 m² compared to a baseline measurement. In some embodiments. the method relates to increasing the subject's eGFR and/or GFR by about 100 mL/min/1.73 m² compared to a baseline measurement.

In some embodiments, the method relates to increasing the subject's eGFR and/or GFR by about 1 mL/min/year compared to a baseline measurement (e.g., SOC). In some embodiments, the method relates to increasing the subject's eGFR and/or GFR by greater than or equal to 1 mL/min/year compared to a baseline measurement. In some embodiments, the method relates to increasing the subject's eGFR and/or GFR by about 2 mL/min/year compared to a baseline measurement. In some embodiments, the method relates to increasing the subject's eGFR and/or GFR by about 3 mL/min/year compared to a baseline measurement. In some embodiments, the method relates to increasing the subject's eGFR and/or GFR by greater than or equal to 3 mL/min/year compared to a baseline measurement. In some embodiments, the method relates to increasing the subject's eGFR and/or GFR by about 5 mL/min/year compared to a baseline measurement. In some embodiments, the method relates to increasing the subject's eGFR and/or GFR by about 7 mL/min/year compared to a baseline measurement. In some embodiments, the method relates to increasing the subject's eGFR and/or GFR by about 9 mL/min/year compared to a baseline measurement. In some embodiments, the method relates to increasing the subject's eGFR and/or GFR by about 10 mL/min/year compared to a baseline measurement. In some embodiments, the method relates to increasing the subject's eGFR and/or GFR by about 15 mL/min/year compared to a baseline measurement. In some embodiments, the method relates to increasing the subject's eGFR and/or GFR by about 20 mL/min/year compared to a baseline measurement. In some embodiments, the method relates to increasing the subject's eGFR and/or GFR by about 25 mL/min/year compared to a baseline measurement. In some embodiments, the method relates to increasing the subject's eGFR and/or GFR by about 30 mL/min/year compared to a baseline measurement. In some embodiments, the method relates to increasing the subject's eGFR and/or GFR by about 35 mL/min/year compared to a baseline measurement. In some embodiments, the method relates to increasing the subject's eGFR and/or GFR by about 40 mL/min/year compared to a baseline measurement. In some embodiments, the method relates to increasing the subject's eGFR and/or GFR by about 45 mL/min/year compared to a baseline measurement. In some embodiments, the method relates to increasing the subject's eGFR and/or GFR by about 50 mL/min/year compared to a baseline measurement. In some embodiments, the method relates to increasing the subject's eGFR and/or GFR by about 55 mL/min/year compared to a baseline measurement. In some embodiments, the method relates to increasing the subject's eGFR and/or GFR by about 60 mL/min/year compared to a baseline measurement. In some embodiments, the method relates to increasing the subject's eGFR and/or GFR by about 65 mL/min/year compared to a baseline measurement. In some embodiments, the method relates to increasing the subject's eGFR and/or GFR by about 70 mL/min/year compared to a baseline measurement. In some embodiments, the method relates to increasing the subject's eGFR and/or GFR by about 75 mL/min/year compared to a baseline measurement. In some embodiments, the method relates to increasing the subject's eGFR and/or GFR by about 80 mL/min/year compared to a baseline measurement. In some embodiments, the method relates to increasing the subject's eGFR and/or GFR by about 85 mL/min/year compared to a baseline measurement. In some embodiments, the method relates to increasing the subject's eGFR and/or GFR by about 90 mL/min/year compared to a baseline measurement. In some embodiments, the method relates to increasing the subject's eGFR and/or GFR by about 95 mL/min/year compared to a baseline measurement. In some embodiments, the method relates to increasing the subject's eGFR and/or GFR by about 100 mL/min/year compared to a baseline measurement. In some embodiments, the method relates to maintaining the subject's eGFR and/or GFR to a level at or near a baseline measurement (e.g., SOC).

In some embodiments, GFR and/or eGFR can be determined by injecting inulin or the inulin-analog sinistrin into plasma. Since both inulin and sinistrin are neither reabsorbed nor secreted by the kidney after glomerular filtration, their rate of excretion is directly proportional to the rate of filtration of water and solutes across the glomerular filter. In some embodiments, GFR and/or eGFR is measured using radioactive substances. In some embodiments, GFR and/or eGFR is measured using chromium-51. In some embodiments, GFR and/or eGFR is measured using renal or plasma clearance of 51Cr-EDTA. In some embodiments, GFR and/or eGFR is measured using technetium-99m. In some embodiments, GFR and/or eGFR is measured using 99mTc-DTPA. A benefit of using radioactive substances is they come close to the ideal properties of inulin (undergoing only glomerular filtration) but can be measured more practically with only a few urine or blood samples. Renal and plasma clearance 51 Cr-EDTA has been shown to be accurate in comparison with inulin. In some embodiments, inulin clearance slightly overestimates glomerular function. In early stage renal disease, inulin clearance may remain normal due to hyperfiltration in the remaining nephrons. Incomplete urine collection is an important source of error in inulin clearance measurement.

Creatinine clearance rate (CCr or CrCl) is the volume of blood plasma that is cleared of creatinine per unit time and is a useful measure for approximating the GFR. Creatinine clearance exceeds GFR due to creatinine secretion, which can be blocked by cimetidine. Both GFR and CCr may be accurately calculated by comparative measurements of substances in the blood and urine, or estimated by formulas using just a blood test result (eGFR and eCCr).

In some embodiments, the disclosure relates to methods of reducing total kidney volume in subject with a renal disease or condition, comprising administering to a subject in need thereof an effective amount of a single-arm ActRIIA heteromultimer or single-arm ActRIIB heteromultimer. In some embodiments, total kidney volume is measured by ultrasound. In some embodiments, total kidney volume is measured by magnetic resonance imaging (MRI). In some embodiments, total kidney volume reflects a sum volume of the kidney and cysts in renal diseases or disorders (e.g., ADPKD). In some embodiments, the method relates to reducing total kidney volume in the subject by at least 2.5% compared to a baseline measurement. In some embodiments, the method relates to reducing total kidney volume in the subject by at least 5% compared to a baseline measurement. In some embodiments, the method relates to reducing total kidney volume in the subject by at least 10% compared to a baseline measurement. In some embodiments, the method relates to reducing total kidney volume in the subject by at least 15% compared to a baseline measurement. In some embodiments, the method relates to reducing total kidney volume in the subject by at least 20% compared to a baseline measurement. In some embodiments, the method relates to reducing total kidney volume in the subject by at least 25% compared to a baseline measurement. In some embodiments, the method relates to reducing total kidney volume in the subject by at least 30% compared to a baseline measurement. In some embodiments, the method relates to reducing total kidney volume in the subject by at least 40% compared to a baseline measurement. In some embodiments, the method relates to reducing total kidney volume in the subject by at least 50% compared to a baseline measurement. In some embodiments, the method relates to reducing total kidney volume in the subject by at least 60% compared to a baseline measurement. In some embodiments, the method relates to reducing total kidney volume in the subject by at least 70% compared to a baseline measurement. In some embodiments, the method relates to reducing total kidney volume in the subject by at least 80% compared to a baseline measurement. In some embodiments, the method relates to reducing total kidney volume in the subject by at least 90% compared to a baseline measurement. In some embodiments, the method relates to reducing total kidney volume in the subject by at least 95% compared to a baseline measurement. In some embodiments, the method relates to reducing total kidney volume in the subject by at least 99% compared to a baseline measurement.

In some embodiments, blood urea nitrogen (BUN) is measured. In some embodiments, a BUN test measures the amount of urea nitrogen in blood. In some embodiments, if kidneys are impaired, the amount of urea nitrogen can be higher. In some embodiments, the disclosure relates to methods of reducing BUN in a subject with a renal disease or condition, comprising administering to a subject in need thereof an effective amount of a single-arm ActRIIA heteromultimer or single-arm ActRIIB heteromultimer. In some embodiments, a normal BUN level for a human is between about 7 mg/dL and about 20 mg/dL. In some embodiments, the method relates to reducing BUN in the subject by at least 2.5% compared to a baseline measurement. In some embodiments, the method relates to reducing BUN in the subject by at least 5% compared to a baseline measurement. In some embodiments, the method relates to reducing BUN in the subject by at least 10% compared to a baseline measurement. In some embodiments, the method relates to reducing BUN in the subject by at least 15% compared to a baseline measurement. In some embodiments, the method relates to reducing BUN in the subject by at least 20% compared to a baseline measurement. In some embodiments, the method relates to reducing BUN in the subject by at least 25% compared to a baseline measurement. In some embodiments, the method relates to reducing BUN in the subject by at least 30% compared to a baseline measurement. In some embodiments, the method relates to reducing BUN in the subject by at least 40% compared to a baseline measurement. In some embodiments, the method relates to reducing BUN in the subject by at least 50% compared to a baseline measurement. In some embodiments, the method relates to reducing BUN in the subject by at least 60% compared to a baseline measurement. In some embodiments, the method relates to reducing BUN in the subject by at least 70% compared to a baseline measurement. In some embodiments, the method relates to reducing BUN in the subject by at least 80% compared to a baseline measurement. In some embodiments, the method relates to reducing BUN in the subject by at least 90% compared to a baseline measurement. In some embodiments, the method relates to reducing BUN in the subject by at least 95% compared to a baseline measurement. In some embodiments, the method relates to reducing BUN in the subject by at least 99% compared to a baseline measurement.

Urine Neutrophil Gelatinase-Associated Lipocalin (NGAL) concentration is an early biomarker of acute kidney injury that is highly sensitive to early injury and is known as a marker of tubular-specific damage. Urine NGAL (or uNGAL) tends to be elevated before serum creatinine levels, allowing for prediction of renal tubular injury. uNGAL increases quantitatively and proportionally according to the severity of renal structural acute kidney injury. In some embodiments, a uNGAL measurement of <50 ng/mL is an indication of low risk of acute kidney injury. In some embodiments, a uNGAL measurement of between about 50 and about 149 ng/mL indicates equivocal risk of acute kidney injury. In some embodiments, a uNGAL measurement of between about 150 and about 300 ng/mL indicates moderate risk of acute kidney injury. In some embodiments, a uNGAL measurement of >300 ng/mL indicates high risk of acute kidney injury.

In some embodiments, the disclosure relates to methods of reducing uNGAL in a subject with a renal disease or condition, comprising administering to a subject in need thereof an effective amount of a single-arm ActRIIA heteromultimer or single-arm ActRIIB heteromultimer. In some embodiments, the method relates to reducing the subject's uNGAL by between about 0.1 and about 50 ng/mL. In some embodiments, the method relates to reducing the subject's uNGAL by between about 0.1 and about 100.0 ng/mL. In some embodiments, the method relates to reducing the subject's uNGAL by between about 0.1 and about 150.0 ng/mL. In some embodiments, the method relates to reducing the subject's uNGAL by between about 0.1 and about 200.0 ng/mL. In some embodiments, the method relates to reducing the subject's uNGAL by between about 0.1 and about 250.0 ng/mL. In some embodiments, the method relates to reducing the subject's uNGAL by between about 0.1 and about 300.0 ng/mL. In some embodiments, the method relates to reducing the subject's uNGAL by between about 0.1 and about 25 ng/mL. In some embodiments, the method relates to reducing the subject's uNGAL by between about 25 and about 50 ng/mL. In some embodiments, the method relates to reducing the subject's uNGAL by between about 50 and about 100 ng/mL. In some embodiments, the method relates to reducing the subject's uNGAL by between about 100 and about 150 ng/mL. In some embodiments, the method relates to reducing the subject's uNGAL by between about 150 and about 200 ng/mL. In some embodiments, the method relates to reducing the subject's uNGAL by between about 200 and about 250 ng/mL. In some embodiments, the method relates to reducing the subject's uNGAL by between about 250 and about 300 ng/mL. In some embodiments, the method relates to reducing the subject's uNGAL by more than 300 ng/mL. In some embodiments, the method relates to reducing the subject's uNGAL by between about 0.1 and about 300 ng/mL.

In some embodiments. the disclosure relates to methods of reducing uNGAL in a subject with a renal disease or condition, comprising administering to a subject in need thereof an effective amount of a single-arm ActRIIA heteromultimer or single-arm ActRIIB heteromultimer. In some embodiments, the method relates to reducing the subject's uNGAL by at least 2.5% compared to a baseline measurement. In some embodiments, the method relates to reducing the subject's uNGAL by at least 5% compared to a baseline measurement. In some embodiments, the method relates to reducing the subject's uNGAL by at least 10% compared to a baseline measurement. In some embodiments, the method relates to reducing the subject's uNGAL by at least 15% compared to a baseline measurement. In some embodiments, the method relates to reducing the subject's uNGAL by at least 20% compared to a baseline measurement. In some embodiments, the method relates to reducing the subject's uNGAL by at least 25% compared to a baseline measurement. In some embodiments, the method relates to reducing the subject's uNGAL by at least 30% compared to a baseline measurement. In some embodiments, the method relates to reducing the subject's uNGAL by at least 40% compared to a baseline measurement. In some embodiments, the method relates to reducing the subject's uNGAL by at least 50% compared to a baseline measurement. In some embodiments, the method relates to reducing the subject's uNGAL by at least 60% compared to a baseline measurement. In some embodiments, the method relates to reducing the subject's uNGAL by at least 70% compared to a baseline measurement. In some embodiments, the method relates to reducing the subject's uNGAL by at least 80% compared to a baseline measurement. In some embodiments, the method relates to reducing the subject's uNGAL by at least 90% compared to a baseline measurement. In some embodiments, the method relates to reducing the subject's uNGAL by at least 95% compared to a baseline measurement. In some embodiments, the method relates to reducing the subject's uNGAL by at least 99% compared to a baseline measurement.

Optionally, methods disclosed herein for treating, preventing, or reducing the progression rate and/or severity of a renal disease or condition (e.g., Alport syndrome, focal segmental glomerulosclerosis (FSGS), polycystic kidney disease, chronic kidney disease), particularly treating, preventing, or reducing the progression rate and/or severity of one or more complications of a renal disease or condition, may further comprise administering to the subject one or more additional active agents and/or supportive therapies for treating a renal disease or condition. In some embodiments, a subject is administered an additional active agent and/or supportive therapy for treating a renal disease or condition (e.g., Alport syndrome, focal segmental glomerulosclerosis (FSGS), polycystic kidney disease, chronic kidney disease). In some embodiments, ARBs and ACE inhibitors are mainstays of therapy for renal diseases and conditions (e.g., Alport syndrome, focal segmental glomerulosclerosis (FSGS), polycystic kidney disease, chronic kidney disease), with beta-blockade and calcium-channel blockers as second-line therapy. In some embodiments, as third-line therapy, thiazides are preferred in subjects with normal renal function, while loop diuretics are preferred in subjects with impaired renal function.

In some embodiments, a subject with a renal disease or condition (e.g., Alport syndrome, focal segmental glomerulosclerosis (FSGS). polycystic kidney disease, chronic kidney disease) is administered an antagonist of the Renin-angiotensin-aldosterone system (RAAS). In some embodiments, RAAS inhibitors include, but are not limited to, angiotensin antagonists (e.g., angiotensin blockade therapy, angiotensin system inhibitor, renin-angiotensin system inhibitor, angiotensin 11 blockade, angiotensin II type 1 receptor blocker, ARB, angiotensin 11 receptor antagonist, AT₁ receptor antagonist, or a sartan) and an angiotensin-converting enzyme (ACE) inhibitor. In some embodiments, RAAS antagonism and particularly, the combination of an ACE inhibitor and ARB, will lower GFR by reducing efferent arteriolar vascular tone and thus, reducing intraglomerular capillary pressure, the driving force for glomerular filtration. Thus, a modest decrease in GFR may be tolerated, providing evidence that RAAS antagonism has been achieved.

In some embodiments, a subject is administered an angiotensin antagonist (e.g., angiotensin receptor blocker. ARB), when the subject shows signs of proteinuria. In some embodiments, an ARB reduces proteinuria in subjects with a renal disease or condition. In some embodiments, an angiotensin antagonist diminishes the rate of glomerulosclerosis in subjects with a renal disease or condition. In some embodiments, administration of an ARB decreases renal disease progression. In some embodiments, a subject is administered one or more ARBs selected from the group consisting of losartan, irbesartan, olmesartan, candesartan, valsartan, fimasartan, azilsartan, salprisartan, and telmisartan. In some embodiments a subject is administered losartan. In some embodiments, a subject is administered irbesartan. In some embodiments, a subject is administered olmesartan. In some embodiments, a subject is administered candesartan. In some embodiments, a subject is administered valsartan. In some embodiments, a subject is administered fimasartan. In some embodiments, a subject is administered azilsartan. In some embodiments, a subject is administered salprisartan. In some embodiments, a subject is administered telmisartan.

In some embodiments, a subject with a renal disease or condition is administered an ACE inhibitor. In some embodiments, an ACE inhibitor is selected from the group consisting of benazepril, captopril, enalapril, lisinopril, perindopril, ramipril (e.g., ramipen), trandolapril, and zofenopril. In some embodiments, a subject is administered benazepril. In some embodiments, a subject is administered captopril. In some embodiments, a subject is administered enalapril. In some embodiments, a subject is administered lisinopril. In some embodiments, a subject is administered perindopril. In some embodiments, a subject is administered ramipril. In some embodiments, a subject is administered trandolapril. In some embodiments, a subject is administered zofenopril. In some embodiments, administration of an ACE inhibitor delays dialysis in a subject with proteinuria and normal kidney function. In some embodiments, administration of an ACE inhibitor slows decline in renal function in a subject. In some embodiments, administration of an ACE inhibitor reduces proteinuria in a subject. In some embodiments, administration of an ACE inhibitor decreases kidney damage in a subject.

In some embodiments, a subject with a renal disease or condition is administered an ARB and an ACE inhibitor. In some embodiments, a subject with a renal disease or condition comprising proteinuria and/or microalbuminuria is administered an ARB and an ACE inhibitor.

In some embodiments, an alternative approach to angiotensin antagonism is to combine an ACE inhibitor and/or ARB with an aldosterone antagonist.

In some embodiments, a subject with a renal disease or condition (e.g., primary FSGS) is administered an immunosuppressive treatment. In some embodiments, subjects with a renal disease or condition are treated with immunosuppressive medications. In some embodiments, immunosuppression is not administered to subjects with secondary FSGS. In some embodiments, immunosuppressants are not administered to subjects that do not have primary FSGS. In some embodiments. an immunosuppressant is selected from the group consisting of corticosteroids, calcineurin inhibitors, janus kinase inhibitors, mammalian target of rapamycin (mTOR) inhibitors, IMDH inhibitors, and biologics (including, but not limited to monoclonal antibodies).

In some embodiments, a subject with a renal disease or condition is administered a corticosteroid. In some embodiments, a glucocorticoid is a corticosteroid. In some embodiments, a subject with a renal disease or condition is administered one or more glucocorticoids. In some embodiments, administration of a glucocorticoid is an initial therapy. In some embodiments, a glucocorticoid is selected from the group consisting of beclomethasone, betamethasone, budesonide. cortisone. dexamethasone, hydrocortisone, methylprednisolone, prednisolone, methylprednisone, prednisone, and triamcinolone. In some embodiments, a subject with a renal disease or condition is administered prednisone. In some embodiments, a subject with a renal disease or condition is administered prednisolone.

In some embodiments, a calcineurin inhibitor is selected from the group consisting of cyclosporine (e.g., cyclosporin, ciclosporin, ciclosporine, Neoral, Sandimmune, SangCya) and tacrolimus (e.g., Astagraf XL, Envarsus XR, Prograf). In some embodiments, calcineurin inhibitors are administered to steroid-sensitive subjects who cannot tolerate continued steroid therapy, and/or to subjects with steroid-resistant renal disease (e.g., steroid-resistant FSGS). In some embodiments, a subject with a renal disease or condition is administered cyclosporine. In some embodiments, a subject with a renal disease or condition is administered tacrolimus.

In some embodiments, a subject with a renal disease or condition maybe administered a combination of one or more corticosteroids and/or calcineurin inhibitors. In some embodiments, a subject with a kidney disease or condition may be administered cyclosporine and prednisone. In some embodiments. a subject with a renal disease or condition is administered tacrolimus and prednisone. In some embodiments, cyclosporine and prednisone are administered to preserve renal function assessed as creatinine clearance.

In some embodiments, treatment with mycophenolate mofetil (MMF) combined with glucocorticoids may be beneficial in subjects who cannot take calcineurin inhibitors. In some embodiments, a subject with a renal disease or condition is administered mycophenolate mofetil (MMF) in combination with one or more glucocorticoids. In some embodiments, a subject with a renal disease or condition is administered MMF and prednisone. In some embodiments, a subject with a renal disease or condition is administered prednisolone and MMF.

In some embodiments, a subject with a renal disease or condition is administered cyclophosphamide and/or prednisone. In some embodiments, a subject with a renal disease or condition is administered prednisolone and/or chlorambucil. In some embodiments, a subject with a renal disease or condition is administered cyclophosphamide. In some embodiments, a subject with a renal disease or condition is administered chlorambucil.

In some embodiments, a janus kinase inhibitor is tofacitinib (e.g., Xeljanz).

In some embodiments, an mTOR inhibitor is selected from the group consisting of sirolimus (e.g., Rapamune) and everolimus (e.g., Afinitor, Zortress).

In some embodiments, an IMDH inhibitor is selected from the group consisting of azathioprine (e.g., Azasan, Imuran), leflunomide (e.g., Arava), and mycophenolate (e.g., CellCept, Myfortic).

In some embodiments, a biologic is selected from the group consisting of abatacept (e.g., Orencia), adalimumab (e.g., Humira), anakinra (e.g., Kineret), basiliximab (e.g., Simulect), certolizumab (e.g., Cimzia), daclizumab (e.g., Zinbryta), etanercept (e.g., Enbrel), fresolimumab, golimumab (e.g., Simponi), infliximab (e.g., Remicade), ixekizumab (e.g., Taltz), natalizumab (e.g., Tysabri), rituximab (e.g., Rituxan), secukinumab (e.g., Cosentyx), tocilizumab (e.g., Actemra), ustekinumab (e.g., Stelara), and vedolizumab (e.g., Entyvio).

In some embodiments, a subject with a renal disease or condition (e.g., Alport syndrome, focal segmental glomerulosclerosis (FSGS), polycystic kidney disease, chronic kidney disease) is administered a statin (e.g., benazepril, valsartan, Fluvastatin, pravastatin).

In some embodiments, a subject with a renal disease or condition (e.g., Alport syndrome, focal segmental glomerulosclerosis (FSGS), polycystic kidney disease, chronic kidney disease) is administered lademirsen. Lademirsen is an anti-miRNA-21.

In some embodiments, a subject with a renal disease or condition (e.g., Alport syndrome, focal segmental glomerulosclerosis (FSGS), polycystic kidney disease, chronic kidney disease) is administered bardoxolone methyl. Bardoxolone methyl is an activator of the KEAPI-Nrf2 pathway and bardoxolone methyl also inhibits the pro-inflammatory transcription factor NF-κB.

In some embodiments, a subject with a renal disease or condition (e.g., Alport syndrome, focal segmental glomerulosclerosis (FSGS), polycystic kidney disease, chronic kidney disease) is administered Achtar gel. Achtar gel was approved in the 1950s by the US Food and Drug Administration for nephrotic syndrome under criteria that were less stringent than required today. In some embodiments, some case studies suggest limited efficacy of Acthar in some subjects with FSGS. In some embodiments, a subject with FSGS is administered Achtar gel.

In some embodiments. a subject with ADPKD is administered Tolvaptan (e.g., OPC-41061). In some embodiments, Tolvaptan has demonstrated a slower decline than placebo in the eGFR over a one year period in patients with late-stage chronic kidney disease but is associated with elevations of bilirubin and alanine aminotransferase levels.

In some embodiments, a subject a renal disease or condition (e.g., Alport syndrome, focal segmental glomerulosclerosis (FSGS). polycystic kidney disease, chronic kidney disease) is administered one or more of abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-0 11, galactose, GC1008, GFB-887, isotretinoin, lanreotide, levamisole. lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, and VX-147.

In some embodiments. a subject with a renal disease or condition (e.g., Alport syndrome, focal segmental glomerulosclerosis (FSGS), polycystic kidney disease, chronic kidney disease) undergoes kidney dialysis. In some embodiments, a subject with a renal disease or condition (e.g., Alport syndrome, focal segmental glomerulosclerosis (FSGS), polycystic kidney disease, chronic kidney disease) undergoes a kidney transplant. In some embodiments, a subject with ESRD undergoes a kidney transplantation. In some embodiments, a subject with a kidney transplant does not experience recurrent renal disease. In some embodiments, a subject with a kidney transplant contracts anti-glomerular basement membrane antibody disease. In some embodiments, anti-glomerular basement membrane antibody disease occurs within one year after kidney transplantation. In some embodiments, a subject with anti-glomerular basement membrane antibody disease is administered methylprednisone and/or cyclophosphamide. In some embodiments, a subject with anti-glomerular basement membrane antibody disease undergoes plasmapheresis.

In some embodiments, a subject with a renal disease or condition (e.g., Alport syndrome, focal segmental glomerulosclerosis (FSGS), polycystic kidney disease, chronic kidney disease) is administered mesenchymal stem cell therapy. In some embodiments, a subject with a renal disease or condition (e.g., Alport syndrome, focal segmental glomerulosclerosis (FSGS), polycystic kidney disease, chronic kidney disease) is administered bone marrow stem cells. In some embodiments, a subject with a renal disease or condition (e.g., Alport syndrome, focal segmental glomerulosclerosis (FSGS), polycystic kidney disease, chronic kidney disease) undergoes lipoprotein removal. In some embodiments, a subject with a renal disease or condition (e.g., Alport syndrome, focal segmental glomerulosclerosis (FSGS), polycystic kidney disease, chronic kidney disease) is administered a Liposorber LA-15 device. In some embodiments, a subject with a renal disease or condition (e.g., Alport syndrome, focal segmental glomerulosclerosis (FSGS), polycystic kidney disease, chronic kidney disease) undergoes plasmapheresis. In some embodiments, a subject with a renal disease or condition (e.g., Alport syndrome, focal segmental glomerulosclerosis (FSGS), polycystic kidney disease, chronic kidney disease) undergoes plasma exchange. In some embodiments, a subject with a renal disease or condition (e.g., Alport syndrome, focal segmental glomerulosclerosis (FSGS), polycystic kidney disease, chronic kidney disease) undergoes a change in diet (e.g., dietary sodium intake).

In some embodiments, methods of the present disclosure delay clinical worsening of a renal disease or condition (e.g., Alport syndrome, focal segmental glomerulosclerosis (FSGS), polycystic kidney disease, chronic kidney disease) in a subject. In some embodiments, methods of the present disclosure reduce the risk of hospitalization for one or more complications associated with a renal disease or condition (e.g., Alport syndrome. focal segmental glomerulosclerosis (FSGS), polycystic kidney disease, chronic kidney disease).

### 7. Pharmaceutical Compositions

In certain aspects, a single-arm ActRIIA heteromultimers or single-arm ActRIIB heteromultimers of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than single-arm ActRIIA heteromultimers or single-arm ActRIIB heteromultimers which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. In some embodiments, a parenteral route of administration is selected from the group consisting of intramuscular, intraperitoneal, intradermal, intravitreal, epidural, intracerebral, intra-arterial, intraarticular, intra-cavernous, intra-lesional, intraosseous, intraocular, intrathecal, intravenous, transdermal, trans-mucosal, extra-amniotic administration, subcutaneous, and combinations thereof. In some embodiments, a parenteral route of administration is subcutaneous. In some embodiments. a parenteral route of administration is a subcutaneous injection. In some embodiments, compositions of the present disclosure are administered by subcutaneous injection. Pharmaceutical compositions suitable for parenteral administration may comprise one or more a single-arm ActRIIA heteromultimers or single-arm ActRIIB heteromultimers in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., a single-arm ActRIIA heteromultimers or single-arm ActRIIB heteromultimers) to a target tissue site, providing a structure for the developing tissue and optimally capable of being resorbed into the body. For example, the matrix may provide slow release of a single-arm ActRIIA heteromultimer or single-arm ActRIIB heteromultimer. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate. and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol: (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds: (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., a single-arm ActRIIA heteromultimer or single-arm ActRIIB heteromultimer). The various factors include, but are not limited to, the subject's age, sex, and diet, the severity disease, time of administration, and other clinical factors. Optionally. the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of single-arm ActRIIA heteromultimers or single-arm ActRIIB heteromultimers. Such therapy would achieve its therapeutic effect by introduction of single-arm ActRIIA heteromultimer or single-arm ActRIIB heteromultimer polynucleotide sequences into cells or tissues having the disorders as listed above. Delivery of a single-arm ActRIIA heteromultimer or single-arm ActRIIB heteromultimer polynucleotide sequences can be achieved using a recombinant expression vector such as a chimeric virus or a colloidal dispersion system. Preferred for therapeutic delivery of a single-arm ActRIIA heteromultimer or single-arm ActRIIB heteromultimer polynucleotide sequences is the use of targeted liposomes.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing an a single-arm ActRIIA heteromultimer or single-arm ActRIIB heteromultimer. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for single-arm ActRIIA heteromultimer or single-arm ActRIIB heteromultimer polynucleotides, is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

It is understood that the dosage regimen will be determined by an attending physician considering various factors which modify the action of the subject compounds of the disclosure (e.g., single-arm ActRIIA heteromultimers or single-arm ActRIIB heteromultimers). The various factors include, but are not limited to, the patient's age, sex. and/or diet, the severity disease, time of administration, and/or other clinical factors. Optionally, the dosage may vary with the type of matrix used in the reconstitution and/or the types of compounds in the composition. The addition of other known growth factors to the final composition. may also affect the dosage.

In some embodiments, one or more single-arm ActRIIA heteromultimers or single-arm ActRIIB heteromultimers of the present disclosure are administered in one or more doses. In some embodiments, a dose of one or more single-arm ActRIIA heteromultimers or single-arm ActRIIB heteromultimers comprises 0.25 mg/kg of the heteromultimer(s). In some embodiments, a dose of one or more single-arm ActRIIA heteromultimers or single-arm ActRIIB heteromultimers comprises 0.50 mg/kg of the heteromultimer(s). In some embodiments, a dose of one or more single-arm ActRIIA heteromultimers or single-arm ActRIIB heteromultimers comprises 0.75 mg/kg of the heteromultimer(s). In some embodiments, a dose of one or more single-arm ActRIIA heteromultimers or single-arm ActRIIB heteromultimers comprises 1.00 mg/kg of the heteromultimer(s). In some embodiments, a dose of one or more single-arm ActRIIA heteromultimers or single-arm ActRIIB heteromultimers comprises 1.25 mg/kg of the heteromultimer(s). In some embodiments, a dose of one or more single-arm ActRIIA heteromultimers or single-arm ActRIIB heteromultimers comprises 1.50 mg/kg of the heteromultimer(s). In some embodiments, a dose of one or more single-arm ActRIIA heteromultimers or single-arm ActRIIB heteromultimers comprises 1.75 mg/kg of the heteromultimer(s). In some embodiments, a dose of one or more single-arm ActRIIA heteromultimers or single-arm ActRIIB heteromultimers comprises 2.00 mg/kg of the heteromultimer(s). In some embodiments, a dose of one or more single-arm ActRIIA heteromultimers or single-arm ActRIIB heteromultimers comprises 2.25 mg/kg of the heteromultimer(s). In some embodiments, a dose of one or more single-arm ActRIIA heteromultimers or single-arm ActRIIB heteromultimers comprises 2.50 mg/kg of the heteromultimer(s). In some embodiments, a dose of one or more single-arm ActRIIA heteromultimers or single-arm ActRIIB heteromultimers comprises 2.75 mg/kg of the heteromultimer(s). In some embodiments, a dose of one or more single-arm ActRIIA heteromultimers or single-arm ActRIIB heteromultimers comprises 3.00 mg/kg of the heteromultimer(s). In some embodiments, a dose of one or more single-arm ActRIIA heteromultimers or single-arm ActRIIB heteromultimers comprises 3.25 mg/kg of the heteromultimer(s). In some embodiments, a dose of one or more single-arm ActRIIA heteromultimers or single-arm ActRIIB heteromultimers comprises 3.50 mg/kg of the heteromultimer(s). In some embodiments, a dose of one or more single-arm ActRIIA heteromultimers or single-arm ActRIIB heteromultimers comprises 3.75 mg/kg of the heteromultimer(s). In some embodiments, a dose of one or more single-arm ActRIIA heteromultimers or single-arm ActRIIB heteromultimers comprises 4.00 mg/kg of the heteromultimer(s). In some embodiments, a dose of one or more single-arm ActRIIA heteromultimers or single-arm ActRIIB heteromultimers comprises 4.25 mg/kg of the heteromultimer(s). In some embodiments, a dose of one or more single-arm ActRIIA heteromultimers or single-arm ActRIIB heteromultimers comprises 4.50 mg/kg of the heteromultimer(s). In some embodiments, a dose of one or more single-arm ActRIIA heteromultimers or single-arm ActRIIB heteromultimers comprises 4.75 mg/kg of the heteromultimer(s). In some embodiments, a dose of one or more single-arm ActRIIA heteromultimers or single-arm ActRIIB heteromultimers comprises 5.00 mg/kg of the heteromultimer(s). In some embodiments, a dose of one or more single-arm ActRIIA heteromultimers or single-arm ActRIIB heteromultimers comprises 5.00 mg/kg of the heteromultimer(s). In some embodiments, a dose of one or more single-arm ActRIIA heteromultimers or single-arm ActRIIB heteromultimers comprises 6.00 mg/kg of the heteromultimer(s). In some embodiments, a dose of one or more single-arm ActRIIA heteromultimers or single-arm ActRIIB heteromultimers comprises 7.00 mg/kg of the heteromultimer(s). In some embodiments, a dose of one or more single-arm ActRIIA heteromultimers or single-arm ActRIIB heteromultimers comprises 8.00 mg/kg of the heteromultimer(s). In some embodiments, a dose of one or more single-arm ActRIIA heteromultimers or single-arm ActRIIB heteromultimers comprises 9.00 mg/kg of the heteromultimer(s). In some embodiments, a dose of one or more single-arm ActRIIA heteromultimers or single-arm ActRIIB heteromultimers comprises 10.00 mg/kg of the heteromultimer(s). In some embodiments, a dose of one or more single-arm ActRIIA heteromultimers or single-arm ActRIIB heteromultimers comprises 20.00 mg/kg of the heteromultimer(s). In some embodiments, a dose of one or more single-arm ActRIIA heteromultimers or single-arm ActRIIB heteromultimers comprises 30.00 mg/kg of the heteromultimer(s). In some embodiments, a dose of one or more single-arm ActRIIA heteromultimers or single-arm ActRIIB heteromultimers comprises at least 0.25 mg/kg of the heteromultimer(s). In some embodiments, a dose of one or more TβRII fusion antagonists comprises between about 0.25 mg/to about 30.00 mg/kg of the heteromultimer(s).

In some embodiments, a dose of one or more single-arm ActRIIA heteromultimers or single-arm ActRIIB heteromultimers of the present disclosure is administered once every day. In some embodiments, a dose of one or more single-arm ActRIIA heteromultimers or single-arm ActRIIB heteromultimers of the present disclosure is administered once every two days. In some embodiments, a dose of one or more single-arm ActRIIA heteromultimers or single-arm ActRIIB heteromultimers of the present disclosure is administered once every three days. In some embodiments, a dose of one or more single-arm ActRIIA heteromultimers or single-arm ActRIIB heteromultimers of the present disclosure is administered once every four days. In some embodiments, a dose of one or more single-arm ActRIIA heteromultimers or single-arm ActRIIB heteromultimers of the present disclosure is administered once every five days. In some embodiments, a dose of one or more single-arm ActRIIA heteromultimers or single-arm ActRIIB heteromultimers of the present disclosure is administered once every six days. In some embodiments, a dose of one or more single-arm ActRIIA heteromultimers or single-arm ActRIIB heteromultimers of the present disclosure is administered once every week. In some embodiments, a dose of one or more single-arm ActRIIA heteromultimers or single-arm ActRIIB heteromultimers of the present disclosure is administered once every two weeks. In some embodiments, a dose of one or more single-arm ActRIIA heteromultimers or single-arm ActRIIB heteromultimers of the present disclosure is administered once every three weeks. In some embodiments, a dose of one or more single-arm ActRIIA heteromultimers or single-arm ActRIIB heteromultimers of the present disclosure is administered once every four weeks. In some embodiments, a dose of one or more single-arm ActRIIA heteromultimers or single-arm ActRIIB heteromultimers of the present disclosure is administered once every other week. In some embodiments. a dose of one or more single-arm ActRIIA heteromultimers or single-arm ActRIIB heteromultimers of the present disclosure is administered once every month. In some embodiments, a dose of one or more single-arm ActRIIA heteromultimers or single-arm ActRIIB heteromultimers of the present disclosure is administered once every two months. In some embodiments, a dose of one or more single-arm ActRIIA heteromultimers or single-arm ActRIIB heteromultimers of the present disclosure is administered once every three months. In some embodiments, a dose of one or more single-arm ActRIIA heteromultimers or single-arm ActRIIB heteromultimers of the present disclosure is administered once every four months. In some embodiments, a dose of one or more single-arm ActRIIA heteromultimers or single-arm ActRIIB heteromultimers of the present disclosure is administered once every five months. In some embodiments, a dose of one or more single-arm ActRIIA heteromultimers or single-arm ActRIIB heteromultimers of the present disclosure is administered once every six months. In some embodiments, a dose of one or more single-arm ActRIIA heteromultimers or single-arm ActRIIB heteromultimers of the present disclosure is administered once every year.

In some embodiments, a dose of one or more single-arm ActRIIA heteromultimers or single-arm ActRIIB heteromultimers of the present disclosure is administered twice every day. In some embodiments. a dose of one or more single-arm ActRIIA heteromultimers or single-arm ActRIIB heteromultimers of the present disclosure is administered twice every two days. In some embodiments, a dose of one or more single-arm ActRIIA heteromultimers or single-arm ActRIIB heteromultimers of the present disclosure is administered twice every three days. In some embodiments, a dose of one or more single-arm ActRIIA heteromultimers or single-arm ActRIIB heteromultimers of the present disclosure is administered twice every four days. In some embodiments, a dose of one or more single-arm ActRIIA heteromultimers or single-arm ActRIIB heteromultimers of the present disclosure is administered twice every five days. In some embodiments, a dose of one or more single-arm ActRIIA heteromultimers or single-arm ActRIIB heteromultimers of the present disclosure is administered twice every six days. In some embodiments, a dose of one or more single-arm ActRIIA heteromultimers or single-arm ActRIIB heteromultimers of the present disclosure is administered twice every week. In some embodiments, a dose of one or more single-arm ActRIIA heteromultimers or single-arm ActRIIB heteromultimers of the present disclosure is administered twice every two weeks. In some embodiments, a dose of one or more single-arm ActRIIA heteromultimers or single-arm ActRIIB heteromultimers of the present disclosure is administered twice every three weeks. In some embodiments, a dose of one or more single-arm ActRIIA heteromultimers or single-arm ActRIIB heteromultimers of the present disclosure is administered twice every four weeks. In some embodiments, a dose of one or more single-arm ActRIIA heteromultimers or single-arm ActRIIB heteromultimers of the present disclosure is administered twice every other week. In some embodiments, a dose of one or more single-arm ActRIIA heteromultimers or single-arm ActRIIB heteromultimers of the present disclosure is administered twice every month. In some embodiments, a dose of one or more single-arm ActRIIA heteromultimers or single-arm ActRIIB heteromultimers of the present disclosure is administered twice every two months. In some embodiments, a dose of one or more single-arm ActRIIA heteromultimers or single-arm ActRIIB heteromultimers of the present disclosure is administered twice every three months. In some embodiments, a dose of one or more single-arm ActRIIA heteromultimers or single-arm ActRIIB heteromultimers of the present disclosure is administered twice every four months. In some embodiments, a dose of one or more single-arm ActRIIA heteromultimers or single-arm ActRIIB heteromultimers of the present disclosure is administered twice every five months. In some embodiments, a dose of one or more single-arm ActRIIA heteromultimers or single-arm ActRIIB heteromultimers of the present disclosure is administered twice every six months. In some embodiments, a dose of one or more single-arm ActRIIA heteromultimers or single-arm ActRIIB heteromultimers of the present disclosure is administered twice every year.

In some embodiments, a dose of one or more single-arm ActRIIA heteromultimers or single-arm ActRIIB heteromultimers of the present disclosure is administered three times every day. In some embodiments, a dose of one or more single-arm ActRIIA heteromultimers or single-arm ActRIIB heteromultimers of the present disclosure is administered three times every two days. In some embodiments, a dose of one or more single-arm ActRIIA heteromultimers or single-arm ActRIIB heteromultimers of the present disclosure is administered three times every three days. In some embodiments, a dose of one or more single-arm ActRIIA heteromultimers or single-arm ActRIIB heteromultimers of the present disclosure is administered three times every four days. In some embodiments, a dose of one or more single-arm ActRIIA heteromultimers or single-arm ActRIIB heteromultimers of the present disclosure is administered three times every five days. In some embodiments, a dose of one or more single-arm ActRIIA heteromultimers or single-arm ActRIIB heteromultimers of the present disclosure is administered three times every six days. In some embodiments, a dose of one or more single-arm ActRIIA heteromultimers or single-arm ActRIIB heteromultimers of the present disclosure is administered three times every week. In some embodiments, a dose of one or more single-arm ActRIIA heteromultimers or single-arm ActRIIB heteromultimers of the present disclosure is administered three times every two weeks. In some embodiments, a dose of one or more single-arm ActRIIA heteromultimers or single-arm ActRIIB heteromultimers of the present disclosure is administered three times every three weeks. In some embodiments, a dose of one or more single-arm ActRIIA heteromultimers or single-arm ActRIIB heteromultimers of the present disclosure is administered three times every four weeks. In some embodiments, a dose of one or more single-arm ActRIIA heteromultimers or single-arm ActRIIB heteromultimers of the present disclosure is administered three times every other week. In some embodiments, a dose of one or more single-arm ActRIIA heteromultimers or single-arm ActRIIB heteromultimers of the present disclosure is administered three times every month. In some embodiments, a dose of one or more single-arm ActRIIA heteromultimers or single-arm ActRIIB heteromultimers of the present disclosure is administered three times every two months. In some embodiments, a dose of one or more single-arm ActRIIA heteromultimers or single-arm ActRIIB heteromultimers of the present disclosure is administered three times every three months. In some embodiments, a dose of one or more single-arm ActRIIA heteromultimers or single-arm ActRIIB heteromultimers of the present disclosure is administered three times every four months. In some embodiments, a dose of one or more single-arm ActRIIA heteromultimers or single-arm ActRIIB heteromultimers of the present disclosure is administered three times every five months. In some embodiments, a dose of one or more single-arm ActRIIA heteromultimers or single-arm ActRIIB heteromultimers of the present disclosure is administered three times every six months. In some embodiments, a dose of one or more single-arm ActRIIA heteromultimers or single-arm ActRIIB heteromultimers of the present disclosure is administered three times every year.

In some embodiments, the present disclosure provides methods of treating renal diseases or conditions, comprising administering a single-arm ActRIIB heteromultimer to a subject in need thereof, wherein the single-arm ActRIIB heteromultimer is administered in a dose of between about 0.25 mg/kg and about 30.00 mg/kg to a subject in need thereof. In some embodiments, the single-arm ActRIIB heteromultimer is administered at least once every week. In some embodiments, the single-arm ActRIIB heteromultimer is administered at least once every three weeks. In some embodiments, the single-arm ActRIIB heteromultimer is administered at least once every four weeks. In some embodiments, the single-arm ActRIIB heteromultimer is administered subcutaneously.

### EXEMPLIFICATION

The invention now being generally described, it will be more readily understood by reference to the following examples, which are included merely for purposes of illustration of certain embodiments of the present invention, and are not intended to limit the invention.

### Example 1. Generation and Characterization of a Single-Arm ActRIIB Heterodimer Fc Fusion

Applicants constructed a soluble single-arm ActRIIB heterodimer Fc fusion comprising a constant region from an IgG heavy chain (e.g., Fc domain) with a short N-terminal extension and a second polypeptide in which the extracellular domain of human ActRIIB was fused to a separate constant region from an IgG heavy chain (e.g., Fc domain) with a linker positioned between the extracellular domain and this second constant region from an IgG heavy chain (e.g., Fc domain). The individual constructs are referred to as monomeric Fc polypeptide and single-arm ActRIIB Fc fusion monomer, respectively, and the sequences for each are provided below.

A methodology for promoting formation of single-arm ActRIIB heterodimer Fc fusions rather than ActRIIB homodimer Fc fusions or Fc homodimeric fusions is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the single-arm ActRIIB Fc fusion monomer and monomeric Fc polypeptide sequences of SEQ ID NOs: 46-48, 84 and 49-51, 85, respectively, one Fe domain is altered to introduce cationic amino acids at the interaction face, while the other Fe domain is altered to introduce anionic amino acids at the interaction face. The single-arm ActRIIB Fc fusion monomer and monomeric Fc polypeptide each employ the tissue plasminogen activator (TPA) leader: MDAMKRGLCCVLLLCGAVFVSP (SEQ ID NO: 45).

The single-arm ActRIIB Fc fusion monomer sequence (SEQ ID NO: 46) is shown below:

(SEQ ID NO: 46)

| | |
|---|---|
| 1 | MDAMKRGLCC VLLLCGAVEV SPGASGRGEA ETRECIYYNA |
| | NWELERTNQS |
| 51 | GLERCEGEQD KRLHCYASWR NSSGTIELVK KGCWLDDFNC |
| | YDRQECVATE |
| 101 | ENPQVYFCCC EGNFCNERFT HLPEAGGPEV TYEPPPTAPT |
| | GGGTHTCPPC |
| 151 | PAPELLGGPS VFLFPPKPKD TLMISRTPEV TCVVVDVSHE |
| | DPEVKFNWYV |
| 201 | DGVEVHNAKT KPREEQYNST YRVVSVLTVL HQDWLNGKEY |
| | KCKVSNKALP |
| 251 | APIEKTISKA KGQPREPQVY TLPPSRKEMT KNQVSLTCLV |
| | KGFYPSDIAV |
| 301 | EWESNGQPEN NYKTTPPVLK SDGSFFLYSK LTVDKSRWQQ |
| | GNVFSCSVMH |
| 351 | EALHNHYTQK SLSLSPGK |

The leader (signal) sequence and linker are underlined. To promote formation of the single-arm ActRIIB heterodimer Fc fusion rather than either of the possible homodimeric complexes (ActRIIB homodimer Fe fusion or homodimer Fc fusion), two amino acid substitutions (replacing acidic amino acids with lysine) can be introduced into the Fc domain of the ActRIIB fusion protein as indicated by double underline above. The amino acid sequence of SEQ ID NO: 46 may optionally be provided with the C-terminal lysine (K) removed.

This single-arm ActRIIB Fc fusion monomer is encoded by the following nucleic acid sequence (SEQ ID NO: 47):

```
(SEQ ID NO: 47)
      1 ATGGATGCAA TGAAGAGAGG GCTCTGCTGT GTGCTGCTGC TGTGTGGAGC
    51 AGTCTTCGTT TCGCCCGGCG CCTCTGGGCG TGGGGAGGCT GAGACACGGG
  101 AGTGCATCTA CTACAACGCC AACTGGGAGC TGGAGCGCAC CAACCAGAGC
  151 GGCCTGGAGC GCTGCGAAGG CGAGCAGGAC AAGCGGCTGC ACTGCTACGC
  201 CTCCTGGCGC AACAGCTCTG GCACCATCGA GCTCGTGAAG AAGGGCTGCT
  251 GGCTAGATGA CTTCAACTGC TACGATAGGC AGGAGTGTGT GGCCACTGAG
  301 GAGAACCCCC AGGTGTACTT CTGCTGCTGT GAAGGCAACT TCTGCAACGA
  351 GCGCTTCACT CATTTGCCAG AGGCTGGGGG CCCGGAAGTC ACGTACGAGC
  401 CACCCCCGAC agcccccacc GGTGGTGGAA CTCACACATG CCCACCGTGG
  451 CCAGCACCTG AACTCCTGGG GGGACCGTCA GTCTTCCTCT TCCCCCCAAA
  501 ACCCAAGGAC ACCCTCATGA TCTCCCGGAC CCCTGAGGTC ACATGCGTGG
  601 TGGTGGACGT GAGCCACGAA GACCCTGAGG TCAAGTTCAA CTGGTACGTG
  601 GACGGCGTGG AGGTGCATAA TGCCAAGACA AAGCCGCGGG AGGAGCAGTA
  651 CAACAGCACG TACCGTGTGG TOAGCGTCOT CACCGTCCTG CACCAGGACT
  701 GGCTGAATGG CAAGGAGTAC AAGTGCAAGG TCTCCAACAA AGCCCTCCCA
  751 GCCCCCATCG AGAAAACCAT CTCCAAAGCC AAAGGGCAGC CCCGAGAACC
  801 ACAGGTGTAC ACCCTGCCCC CATCCCGGAA GGAGATGACC AAGAACCAGG
  851 TCAGCCTGAC CTGCCTGGTC AAAGGCTTCT ATCCCAGCGA CATCGCCGTG
  901 GAGTGGGAGA GCAATGGGCA GCCGGAGAAC AACTACAAGA CCACGCCTCC
  951 CGTGCTGAAG TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA
```

A mature single-arm ActRIIB Fe fusion monomer (SEQ ID NO: 48) is as follows.

```
(SEQ ID NO: 48)
    1 GRGEAETREC IYYNANWELE RTNQSGLERC
        EGEQDKRLHC YASWRNSSGT
  51 IELVKKGCWL DDFNCYDRQE CVATEENPQV
        YFCCCEGNFC NERFTHLPEA
101 GGPEVTYEPP PTAPTGGGTH TCPPCPAPEL
        LGGPSVFLFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVYTLPPS
251 RKEMTKNQVS LTCLVKGFYP SDIAVEWESN
        GQPENNYKTT PPVLKSDGSE
301 FLYSKLTVDK SRWQQGNVFS CSVMHEALHN
        HYTQKSLSLS PGK
```

A mature single-arm ActRIIB Fc fusion monomer (SEQ ID NO: 84) may optionally be provided with the C-terminal lysine removed, as depicted below.

```
(SEQ ID NO: 84)
    1 GRGEAETREC IYYNANWELE RTNQSGLERC
        EGEQDKRLHC YASWRNSSGT
  51 IELVKKGCWL DDFNCYDRQE CVATEENPQV
        YFCCCEGNFC NERFTHLPEA
101 GGPEVTYEPP PTAPTGGGTH TCPPCPAPEL
        LGGPSVFLFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVYTLPPS
251 RKEMTKNQVS LTCLVKGFYP SDIAVEWESN
        GQPENNYKTT PFVLKSDGSF
301 FLYSKLTVDK SRWQQGNVFS CSVMHEALHN
        HYTQKSLSLS PG
```

The complementary human G1Fc polypeptide (SEQ ID NO: 49) employs the TPA leader and is as follows:

(SEQ ID NO: 49)

| | |
|---|---|
| 1 | MDAMKRGLCC VLLLCGAVFV SPGASNTKVD KRVTGGGTHT |
| | CPPCPAPELL |
| 51 | GGPSVFLFPP KPKDTLMISR TPEVTCVVVD VSHEDPEVKF |
| | NWYVDGVEVH |
| 101 | NAKTKPREEQ YNSTYRVVSV LTVLHQDWLN GKEYKCKVSN |
| | KALPAPIEKT |
| 151 | ISKAKGQPRE PQVYTLPPSR EEMTKNQVSL TCLVKGFYPS |
| | DIAVEWESNG |
| 201 | QPENNYDTTP PVLDSDGSFF LYSDLTVDKS RWQQGNVFSC |
| | SVMHEALHNH |
| 251 | YTQKSLSLSP GK |

The leader sequence is underlined, and an optional N-terminal extension of the Fc polypeptide is indicated by double underlined. To promote formation of the single-arm ActRIIB heterodimer Fc fusion rather than either of the possible homodimeric fusions, two amino acid substitutions (replacing lysines with anionic residues) can be introduced into the monomeric Fc polypeptide as indicated by double underline above. The amino acid sequence of SEQ ID NO: 49 may optionally be provided with the C-terminal lysine removed.

This complementary Fc polypeptide is encoded by the following nucleic acid (SEQ ID NO: 50).

```
(SEQ ID NO: 50)
    1 ATGGATGCAA TGAAGAGAGG GCTCTGCTGT
        GTGCTGCTGC TGTGTGGAGC
  51 AGTCTTCGTT TCGCCCGGCG CCAGCAACAC
        CAAGGTGGAC AAGAGAGTTA
101 CCGGTGGTGG AACTCACACA TGCCCACCGT
        GCCCAGCACC TGAACTCCTG
151 GGGGGACCGT CAGTCTTCCT CTTCCCCCCA
        AAACCCAAGG ACACCCTCAT
201 GATCTCCCGG ACCCCTGAGG TCACATGCGT
        GGTGGTGGAC GTGAGCCACG
251 AAGACCCTGA GGTCAAGTTC AACTGGTACG
        TGGACGGCGT GGAGGTGCAT
301 AATGCCAAGA CAAAGCCGCG GGAGGAGCAG
        TACAACAGCA CGTACCGTGT
351 GGTCAGCGTC CTCACCGTCC TGCACCAGGA
        CTGGCTGAAT GGCAAGGAGT
401 ACAAGTGCAA GGTCTCCAAC AAAGCCCTCC
        CAGCCCCCAT CGAGAAAACC
451 ATCTCCAAAG CCAAAGGGCA GCCCCGAGAA
        CCACAGGTGT ACACCCTGCC
501 CCCATCCCGG GAGGAGATGA CCAAGAACCA
        GGTCAGCCTG ACCTGCCTGG
551 TCAAAGGCTT CTATCCCAGC GACATCGCCG
        1GGAGTGGGA GAGCAATGGG
601 CAGCCGGAGA ACAACTACGA CACCACGCCT
        CCCGTGCTGG ACTCCGACGG
651 CTCCTTCTTC CTCTATAGCG ACCTCACCGT
        GGACAAGAGC AGGTGGCAGC
701 AGGGGAACGT CTTCTCATGC TCCGTGATGC
        ATGAGGCTCT GCACAACCAC
751 TACACGCAGA AGAGCCTCTC CCTGTCTCCG
        GGTAAA
```

The sequence of a mature monomeric Fe polypeptide is as follows (SEQ ID NO: 51).

```
(SEQ ID NO: 51)
    1 SNTKVDKRVT GGGTHTCPPC PAPELLGGPS
        VELFPPKPKD TLMISRTPEV
  51 TCVVVDVSHE DPEVKFNWYV DGVEVHNAKT
        KPREEOYNST YRVVSVLTVI
101 HQDWLNGKEY KCKVSNKALP APIEKTISKA
        KGQPREPQVY TLPPSREEMT
151 KNQVSLTCLV KGFYPSDIAV EWESNGQPEN
        NYDTTPPVLD SDGSFFLYSD
201 LTVDKSRWQQ GNVFSCSVMH EALHNHYTQK
        SLSLSPGK
```

The sequence of a mature monomeric Fe polypeptide (SEQ ID NO: 85) may optionally be provided with the C-terminal lysine removed, as depicted below.

```
(SEQ ID NO: 85)
    1 SNTKVDKRVT GGGTHTCPPC PAPELLGGPS
        VFLFPFKPKD TLMISRTPEV
  51 TCVVVDVSHE DPEVKFNWYV DGVEVHNAKT
        KPREEQYNST YRVVSVLTVL
101 HQDWLNGKEY KCKVSNKALP APIEKTISKA
        KGQPREPQVY TLPPSREEM
151 KNQVSLTCLV KGFYPSDIAV EWESNGQPEN
        NYDTTPPVLD SDGSFFLYSD
201 LTVDKSRWQQ GNVFSCSVMH EALHMHYTQK
        SLSLSPG
```

The single-arm ActRIIB Fc fusion monomer and monomeric Fc polypeptide of SEQ ID NO: 48 (or SEQ ID NO: 84) and SEQ ID NO: 51 (or SEQ ID NO: 85), respectively, may be co-expressed and purified from a CHO cell line to give rise to a single-arm ActRIIB heterodimer Fc fusion.

In another approach to promote the formation of heteromultimers using asymmetric Fc fusion polypeptides, the Fc domains are altered to introduce complementary hydrophobic interactions and an additional intermolecular disulfide bond, as illustrated in the single-arm ActRIIB Fc fusion monomer and monomeric Fc polypeptide sequences of SEQ ID NOs: 60-61, 86, 90-91, and 62-63, 87, respectively.

The single-arm ActRIIB Fc fusion monomer sequence (SEQ ID NO: 60) employs the TPA leader and is shown below:

(SEQ ID NO: 60)

| | |
|---|---|
| 1 | MDAMKRGLCC VLLLCGAVFV SPGASGRGEA ETRECIYYNA |
| | NWELERITNQS |
| 51 | GLERCEGEQD KRLHCYASWR NSSGTIELVK KGCWLDDFNC |
| | YDRQECVATE |
| 101 | ENPQVYFCCC EGNFCNERFT HLPEAGGPEV TYEPPPTAPT |
| | GGGTHTCPPC |
| 151 | PAPELLGGPS VFLFPPKPKD TLMISRTPEV TCVVVDVSHE |
| | DPEVKFNWYV |
| 201 | DGVEVHNAKT KPREEQYNST YRVVSVLTVL HQDWLNGKEY |
| | KCKVSNKALP |
| 251 | APIEKTISKA KGQPREPQVY TLPPCREEMT KNQVSLWCLV |
| | KGFYPSDIAV |
| 301 | EWESNGQPEN NYKTTPPVLD SDGSFFLYSK LTVDKSRWQQ |
| | GNVFSCSVMH |
| 351 | EALHNHYTQK SLSLSPGK |

The leader sequence and linker are underlined. To promote formation of the single-arm ActRIIB heterodimer Fc fusion rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 60 may optionally be provided with the C-terminal lysine removed.

A mature single-arm ActRIIB Fc fusion monomer is as follows:

```
(SEQ ID NO: 61)
    1 GRGEAETREC IYYNANWELE RTNQSGLERC
        EGEQDKRLHC YASWRNSSGT
  51 IELVKKGCWL DDFNCYDRQE CVATEENPQV
        YFCCCEGNFC NERFTHLPEA
101 GGPEVTYEPP PTAPTGGGTH TCPPCPAPEL
        LGGPSVFLFP PKPKDTLMIS
151 RTPEVTCVVV DVSHEDPEVK FNWYVDGVEV
        HNAKTKPREE QYNSTYRVVS
201 VLTVLHQDWL NGKEYKCKVS NKALPAPIEK
        TISKAKGQPR EPQVYTLPPC
251 REEMTKNQVS LWCLVKGFYP SDIAVEWESN
        GQPENNYKTT PPVLDSDGSF
301 FLYSKLTVDK SRWQQGNVFS CSVMHEALHN
        HYTQKSLSLS PGK
```

An alternative mature single-arm ActRIIB Fc fusion monomer is as follows:

```
(SEQ ID NO: 90)
    1 SGRGEAETRE CIYYNANWEL ERTNQSGLER
        CEGEQDKRDH CYASWRNSSG
  51 TIELVKKGCW LDDFNCYDRQ ECVATEENPQ
        VYFCCCEGNF CNERFTHLPE
101 AGGPEVTYEP PPTAPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRVV
201 SVLTVLHQDW LNGKEYKCKV SNKALPAPIE
        KTISKAKGQP REPQVYTLPP
251 CREEMTKNQV SLWCLVKGFY PSDIAVEWES
        NGQPENNYKT TPPVLDSDGS
301 FFLYSKLTVD KSRWQQGNVF SCSVMHEALH
        NHYTQKSLSL SPGK
```

A mature single-arm ActRIIB Fc fusion monomer may optionally be provided with the C-terminal lysine removed, as depicted below.

```
(SEQ ID NO: 86)
    1 GRGEAETREC IYYNANWELE RTNQSGLERC
        EGEQDKRLHC YASWRNSSGT
  51 IELVKKGCWL DDFNCYDRQE CVATEENPQV
        YFCCCEGNFC NERFTHLPEA
101 GGPEVTYEPP PTAPTGGGTE TCPPCPAPEL
        LGGPSVFLFP PKPKDTLMIS
151 RTPEVTCVVV DVSHEDPEVK FNWYVDGVEV
        HNAKTKPREE QYNSTYRVVS
201 VLTVDHQDWL NGKEYKCKVS NKALPAPIEK
        TISKAKGQPR EPQVYTLPPC
951 REEMTKNQVS LWCLVKGFYP SDIAVEWESN
        GQPENNYKTT PPVLDSDGSE
301 FLYSKLTVDK SRWQQGNVFS CSVMHEALHN
        HYTQKSLSLS PG
```

An alternative mature single-arm ActRIIB Fc fusion monomer is as follows:

```
(SEQ ID NO: 91)
    1 SGRGEAETRE CIYYNANWEL ERTNQSGEER
        CEGEQDKRLH CYASWRNSSG
  51 TIELVKKGCW LDDFNCYDRQ ECVATEENPQ
        VYFCCCEGNF CNERFTHLPE
101 AGGPEVTYEP PPTAPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRVV
201 SVLTVLHQDW LNGKEYKCKV SNKALPAPIE
        KTISKAKGQP REPQVYTLPP
251 CREEMTKNQV SLWCLVKGFY PSDIEIVEWES
        NGQPENNYKT TPPVLDSDGS
301 FFLYSKLTVD KSRWQQGNVF SCSVNIHEALH
        NHYTQKSLSL SPG
```

The complementary form of monomeric Fc polypeptide (SEQ ID NO: 62) uses the TPA leader and is as follows.

(SEQ ID NO: 62)

| | |
|---|---|
| 1 | MDAMKRGLCC VLLLCGAVFV SPGASNTKVD KRVTGGGTHT |
| | CPPCPAPELL |
| 51 | GGPSVFLFPP KPKDTLMISR TPEVTCVVVD VSHEDPEVKF |
| | NWYVDGVEVH |
| 101 | NAKTKPREEQ YNSTYRVVSV LTVLHQDWLN GKEYKCKVSN |
| | KALPAPIEKT |
| 151 | ISKAKGQPRE PQVCTLPPSR EEMTKNQVSL SCAVKGFYPS |
| | DIAVEWESNG |
| 201 | QPENNYKTTP PVLDSDGSFF LVSKLTVDKS RWQQGNVFSC |
| | SVMHEALHNH |
| 251 | YTQKSLSLSP GK |

The leader sequence is underlined, and an optional N-terminal extension of the Fe polypeptide is indicated by double underline. To promote formation of the single-arm ActRIIB heterodimer Fc fusion rather than either of the possible homodimeric complexes, four amino acid substitutions can be introduced into the monomeric Fe polypeptide as indicated by double underline above. The amino acid sequence of SEQ ID NO: 62 may optionally be provided with the C-terminal lysine removed.

A mature monomeric Fe polypeptide sequence (SEQ ID NO: 63) is as follows.

```
(SEO ID NO: 63)
    1 SNTKVDKRVT GGGTHTCPPC PAPELLGGPS
        VELFPPKPKD TLMISRTPEV
  51 TCVVVDVSHE DPEVKFNWYV DGVEVHNAKT
        KPREEQYNST YRVVSVLTVL
101 HQDWLNGKEY KCKVSNKALP APIEKTISKA
        KGQPREPQVC TLPPSREEMI
151 KNQVSLSCAV KGFYPSDIAV EWESNGQPEN
        NYKTTPPVLD SDGSFFLVSK
201 LTVDKSRWQQ GNVFSCSVMH EALHNHYTQK
        SLSLSPGK
```

A mature monomeric Fe polypeptide sequence (SEQ ID NO: 87) may optionally be provided with the C-terminal lysine removed, as depicted below.

```
(SEQ ID NO: 87)
    1 SNTKVDKRVT GGGTHTCPPC PAPELLGGPS
        VFLFPPKPKD TLMISRTPEV
  51 TCVVVDVSHE DPEVKFNWYV DGVEVHNAKT
        KPREEQYNST YKVVSVLTVL
101 HQDWLNGKEY KCKVSNKALP APIEKTISKA
        KGQPREPQVC TLPPSREEMT
151 KNQVSLSCAV KGFYPSDIAV EWESNGQPEN
        NYKTTPPVLD SDGSFFLVSK
201 LTVDKSRWQQ GNVFSCSVMH EALHNHYTQK
        SLSLSPG
```

The single-arm ActRIIB Fc fusion monomer and monomeric Fc polypeptide of SEQ ID NO: 61 (or SEQ ID NO: 86) and SEQ ID NO: 63 (or SEQ ID NO: 87), respectively. may be co-expressed and purified from a CHO cell line to give rise to a single-arm ActRIIB heterodimer Fc fusion.

The single-arm ActRIIB Fc fusion monomer and monomeric Fc polypeptide of SEQ ID NO: 90 (or SEQ ID NO: 91) and SEQ ID NO: 63 (or SEQ ID NO: 87), respectively. may be co-expressed and purified from a CHO cell line to give rise to a single-arm ActRIIB heterodimer Fc fusion.

Purification of various single-arm ActRIIB heterodimer Fc fusions could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to compare ligand binding selectivity of the single-arm ActRIIB heterodimer Fc fusion described above with that of ActRIIB homodimer Fc fusion. Single-arm ActRIIB homodimer Fc fusion and ActRIIB homodimer Fc fusion were independently captured onto the system using an anti-Fc antibody. Ligands were injected and allowed to flow over the captured receptor protein. Results are summarized in the table below, in which ligand off-rates (k_{d}) typically associated with the most effective ligand traps are denoted by gray shading.

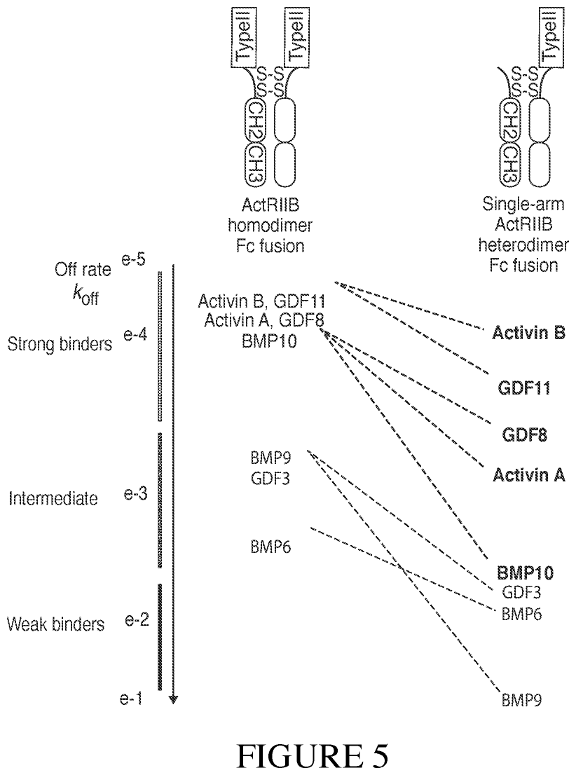

These comparative binding data demonstrate that single-arm ActRIIB heterodimer Fc fusion has greater ligand selectivity than ActRIIB homodimer Fc fusion. Whereas ActRIIB homodimer Fc fusion binds strongly to five important ligands (see cluster of activin A, activin B, BMP10, GDF8, and GDF11 in FIG. 5), single-arm ActRIIB heterodimer Fc fusion discriminates more readily among these ligands. Thus, single-arm ActRIIB heterodimer Fc fusion binds strongly to activin B and GDF11 and with intermediate strength to GDF8 and activin A. In further contrast to ActRIIB homodimer Fc fusion, single-arm ActRIIB heterodimer Fc fusion displays only weak binding to BMP10 and no binding to BMP9. See FIG. 5.

These results indicate that single-arm ActRIIB heterodimer Fc fusion is a more selective antagonist than ActRIIB homodimer Fc fusion. Accordingly. single-arm ActRIIB heterodimer Fc fusion will be more useful than ActRIIB homodimer Fc fusion in certain applications where such selective antagonism is advantageous. Examples include therapeutic applications where it is desirable to retain antagonism of one or more of activin A, activin B, GDF8, and GDF11 but minimize antagonism of one or more of BMP9, BMP10, BMP6, and GDF3. Selective inhibition of ligands in the former group would be particularly advantageous therapeutically because they constitute a subfamily which tends to differ functionally from the latter group and its associated set of clinical conditions.

### Example 2. Generation and Characterization of a Single-Arm ActRIIA Heterodimer Fc Fusion

Applicants constructed a soluble single-arm ActRIIA heterodimer Fc fusion comprising a monomeric Fc polypeptide with a short N-terminal extension and a second polypeptide in which the extracellular domain of human ActRIIA was fused to a separate Fc domain with a linker positioned between the extracellular domain and this second Fc domain. The individual constructs are referred to as monomeric Fc polypeptide and single-arm ActRIIA Fc fusion monomer, respectively, and the sequences for each are provided below.

Formation of a single-arm ActRIIA heterodimer Fc fusion may be guided by approaches similar to those described for single-arm ActRIIB heterodimer Fc fusion in Example 1. In a first approach, illustrated in the single-arm ActRIIA Fc fusion monomer and monomeric Fc polypeptide sequences of SEQ ID NOs: 55-57, 88 and 49-51, 85. respectively, one Fe domain is altered to introduce cationic amino acids at the interaction face, while the other Fc domain is altered to introduce anionic amino acids at the interaction face.

The single-arm ActRIIA Fc fusion monomer employs the TPA leader and is as follows:

(SEQ ID NO: 55)

| | |
|---|---|
| 1 | MDAMKRGLCC VILLLCGAVFV SPGAAILGRS ETQECLFFNA |
| | NWEKDRTNQT |
| 51 | GVEPCYGDKD KRRHCFATWK NISGSIEIVK QGCWLDDINC |
| | YDRTDCVEKK |
| 101 | DSPEVYFCCC EGNMCNEKFS YFPEMEVTQP TSNPVTPKPP |
| | TGGGTHTCPP |
| 151 | CPAPELLGGP SVFLFPPKPK DTLMISRTPE VTCVVVDVSH |
| | EDPEVKFNWY |
| 201 | VDGVEVHNAK TKPREEQYNS TYRVVSVLTV LHQDWLNGKE |
| | YKCKVSNKAL |
| 251 | PAPIEKTISK AKGQPREPQV YTLPPSRKEM TKNQVSLTCL |
| | VKGFYPSDIA |
| 301 | VEWESNGQPE NNYKTTPPVL KSDGSFFLYS KLTVDKSRWQ |
| | QGNVFSCSVM |
| 351 | HEALHNHYTQ KSLSLSPGK |

The leader and linker sequences are underlined. To promote formation of the single-arm ActRIIA heterodimer Fc fusion rather than either of the possible homodimeric complexes (ActRIIA homodimer Fe fusion or Fe homodimeric fusions), two amino acid substitutions (replacing anionic residues with lysines) can be introduced into the Fc domain of the fusion polypeptide as indicated by double underline above. The amino acid sequence of SEQ ID NO: 55 may optionally be provided with the C-terminal lysine removed.

This single-arm ActRIIA Fe fusion monomer is encoded by the following nucleic acid (SEQ ID NO: 56).

```
(SEQ ID NO: 56)
      1 ATGGATGCAA TGAAGAGAGG GCTCTGCTGT
          GTGCTGCTGC TGTGTGGAGC
    51 AGTCTTCGTT TCGCCCGGCG CCGCTATACT
          TGGTAGATCA GAAACTCAGG
  101 AGTGTCTTTT CTTTAATGCT AATTGGGAAA
          AAGACAGAAC CAATCAAACT
  151 GGTGTTGAAC CGTGTTATGG TGACAAAGAT
          AAACGGCGGC ATTGTTTTGC
  201 TACCTGGAAG AATATTTCTG GTTCCATTGA
          AATAGTGAAA CAAGGTTGTT
  251 GGCTGGATGA TATCAACTGC TATGACAGGA
          CTGATTGTGT AGAAAAAAAA
  301 GACAGCCCTG AAGTATATTT CTGTTGCTGT
          GAGGGCAATA TGTGTAATGA
  351 AAAGTTTTCT TATTTTCCGG AGATGGAAGT
          CACACAGCCC ACTTCAAATC
  401 CAGTTACACC TAAGCCACCC ACCGGTGGTG
          GAACTCACAC ATGCCCACCG
  451 TGCCCAGCAC CTGAACTCCT GGGGGGACCG
          TCAGTCTTCC TCTTCCCCCC
  501 AAAACCCAAG GACACCCTCA TGATCTCCCG
          GACCCCTGAG GTCACATGCG
  551 TGGTGGTGGA CGTGAGCCAC GAAGACCCTG
          AGGTCAAGTT CAACTGGTAC
  601 GTGGACGGCG TGGAGGTGCA TAATGCCAAG
          ACAAAGCCGC GGGAGGAGCA
  651 GTAGAACAGC ACGTACCGTG TGGTCAGCGT
          CCTCACCGTC CTGCACCAGG
  701 ACTGGCTGAA TGGCAAGGAG TACAAGTGCA
          AGGTCTCCAA CAAAGCCCTC
  751 CCAGCCCCCA 1CGAGAAAAC CATCTCCAAA
          GCCAAAGGGC AGCCCCGAGA
  801 AGOACAGGTG TACACCCTGC CCCCATCCCG
          GAAGGAGATG ACCAAGAACC
  851 AGGTCAGCCT GACCTGCCTG GTCAAAGGCT
          TCTATCCCAG CGACATCGCC
  901 GTGGAGTGGG AGAGCAATGG GCAGCCGGAG
          AACAACTACA AGACCACGCC
  951 TCCCGTGCTG AAGTCCGACG GCTCCTTCTT
          CCTCTATAGC AAGCTCACCG
1001 TGGACAAGAG CAGGTGGCAG CAGGGGAACG
          TCTTCTCATG CTCCGTGATG
1051 CATGAGGCTC TGCACAACCA CTACACGCAG
          AAGAGCCTCT CCCTGTCTCC
1101 GGGTAAA
```

A mature single-arm ActRIIA Fc fusion monomer sequence is as follows (SEQ ID NO: 57).

```
(SEQ ID NO: 57)
    1 ILGRSETQEC DEFNANWEKD RTNQTGVEPC
        YGDKDKRRHC FATWKNTSGS
  51 IEIVKQGCWL DDINCYDRTD CVEKKDSPEV
        YFCCCEGNMC NEKFSYFPEM
101 EVTQPTSNPV TPKPPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRVV
201 SVLTVLHQDW LNGKEYKCKV SNKALPAPIE
        KTISKAKGQP REPQVYTLPP
251 SRKEMTKNQV SLTCLVKGFY PSDIAVEWES
        NGQPENNYKT TPPVLKSDGS
301 FFLYSKLTVD KSRWQQGNVE SCSVMHEALH
        NHYTQKSLSL SPGK
```

A mature single-arm ActRIIA Fc fusion monomer sequence may optionally be provided with the C-terminal lysine removed as follows (SEQ ID NO: 88).

```
(SEQ ID NO: 88)
    1 ILGRSETQEC LFFNANWEKD RTNQTGVEPC
        YGDKDKRRHC FATWKNISGS
  51 IEIVKQGCWL DDINCYDRTD CVEKKDSPEV
        YFCCCEGNMC NEKFSYFPEM
101 EVTQPTSNPV TPKPPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRVV
201 SVLTVLHQDW LNGKEYKCKV SNKALPAPIE
        KTISKAKGQP REPQVYTLPP
251 SRKEMTKNQV SLTCLVKGFY PSDIAVEWES
        NGQPENNYKT TPPVLKSDGS
301 FFLYSKLTVD KSRWQQGNVF
        SCSVMHEAL
```

As described in Example 1, the complementary form of monomeric human G1Fc polypeptide (SEQ ID NO: 49) employs the TPA leader and incorporates an optional N-terminal extension. To promote formation of the single-arm ActRIIA heterodimer Fc fusion rather than either of the possible homodimeric complexes, two amino acid substitutions (replacing lysines with anionic residues) can be introduced into the monomeric Fc polypeptide. The amino acid sequence of SEQ ID NO: 49 may optionally be provided without the C-terminal lysine. This complementary Fe polypeptide is encoded by the nucleic acid of SEQ ID NO: 50, and a mature monomeric Fc polypeptide (SEQ ID NO: 51 or SEQ ID NO: 85) may optionally be provided with the C-terminal lysine removed.

The single-arm ActRIIA Fc fusion monomer and monomeric Fc polypeptide of SEQ ID NO: 57 (or SEQ ID NO: 88) and SEQ ID NO: 51 (or SEQ ID NO: 85), respectively, may be co-expressed and purified from a CHO cell line to give rise to a single-arm ActRIIA heterodimer Fc fusion.

In another approach to promoting the formation of heteromultimers using asymmetric Fc fusion polypeptides, the Fc domains are altered to introduce complementary hydrophobic interactions and an additional intermolecular disulfide bond as illustrated in the single-arm ActRIIA Fc fusion monomer and Fc polypeptide sequences of SEQ ID NOs: 58-59, 89 and 62-63, 87, respectively.

The single-arm ActRIIA Fc fusion monomer (SEQ ID NO: 58) uses the TPA leader and is as follows:

(SEQ ID NO: 58)

| | |
|---|---|
| 1 | MDAMKRGLCC VLLLCGAVFV SPGAAILGRS ETQECLFFNA |
| | NWEKDRTNQT |
| 51 | GVEPCYGDKD KRRHCFATWK NISGSIEIVK QGCWLDDINC |
| | YDRTDCVEKK |
| 101 | DSPEVYFCCC EGNMCNEKFS YFPEMEVTQP TSNPVTPKPP |
| | TGGGTHTCPP |
| 151 | CPAPELLGGP SVFLFPPKPK DTLMISRTPE VTCVVVDVSH |
| | EDPEVKFNWY |
| 201 | VDGVEVHNAK TKPREEQYNS TYRVVSVLTV LHQDWLNGKE |
| | YKCKVSNKAL |
| 251 | PAPIEKTISK AKGQPREPQV YTLPPCREEM TKNQVSLWCL |
| | VKGFYPSDIA |
| 301 | VEWESNGQPE NNYKTTPPVL DSDGSFFLYS KLTVDKSRWQ |
| | QGNVFSCSVM |
| 351 | HEALHNHYTQ KSLSLSPGK |

The leader sequence and linker are underlined. To promote formation of the single-arm ActRIIA heterodimer Fc fusion rather than either of the possible homodimeric complexes, two amino acid substitutions (replacing a serine with a cysteine and a threonine with a tryptophan) can be introduced into the Fc domain of the single-arm ActRIIA Fc fusion monomer as indicated by double underline above. The amino acid sequence of SEQ ID NO: 58 may optionally be provided with the C-terminal lysine removed (SEQ ID NO: 89).

A mature single-arm ActRIIA Fc fusion monomer (SEQ ID NO: 59) is as follows.

```
(SEQ ID NO: 59)
    1 ILGRSETQEC LFFNANWEKD RTNQTGVEPC
        YGDKDKRRHC FATWKNISGS
  51 IEIVKQGCWI DDINCYDRTD CVEKKDSPEV
        YECCCEGNMC NEKFSYFPEM
101 EVTQPTSNPV TPKPPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRW
201 SVLTVLHQDW LNGKEYKCKV SNKALPAPIE
        KTISKAKGQP REPQVYTLPP
251 CREEMTKNQV SLWCLVKGFY PSDIAVEWES
        NGQPENNYKT TPPVLDSDGS
301 FELYSKLTVD KSRWQQGNVF SCSVMHEALH
        NHYTQKSLSL SPGK
```

A mature single-arm ActRIIA Fc fusion monomer may optionally be provided with the C-terminal lysine removed (SEQ ID NO: 89) as follows.

```
(SEQ ID NO: 89)
    1 ILGRSETQEC LEFNANWEKD RTNQTGVEPC
        YGDKDKRRHC FATWKNISGS
  51 IEIVKQGCWL DDINCYDRTD CVEKKDSPEV
        YFCCCEGNMC NEKFSYFPEM
101 EVTQPTSNPV TPKPPTGGGT HTCPPCPAPE
        LLGGPSVFLF PPKPKDTLMI
151 SRTPEVTCVV VDVSHEDPEV KFNWYVDGVE
        VHNAKTKPRE EQYNSTYRVV
201 SVLTVLHQDW LNGKEYKCKV SNKALPAPIE
        KTISKAKGQP REPQVYTLPP
251 CREEMTKNQV SLWCLVKGFY PSDIAVEWES
        NGQPENNYKT TPPVLDSDGS
301 FFLYSKLTVD KSRWQQGNVF SCSVMHEALH
        NHYTQKSLSL SPG
```

As described in Example 1, the complementary form of monomeric human G1Fc polypeptide (SEQ ID NO: 62) employs the TPA leader and incorporates an optional N-terminal extension. To promote formation of the single-arm ActRIIA heterodimer Fc fusion rather than either of the possible homodimeric complexes, four amino acid substitutions can be introduced into the monomeric Fc polypeptide as indicated. The amino acid sequence of SEQ ID NO: 62 and a mature G1Fc polypeptide (SEQ ID NO: 63) may optionally be provided with the C-terminal lysine removed (SEQ ID NO: 87).

The single-arm ActRIIA Fc fusion monomer and monomeric Fc polypeptide of SEQ ID NO: 59 (or SEQ ID NO: 89) and SEQ ID NO: 63 (or SEQ ID NO: 87), respectively. may be co-expressed and purified from a CHO cell line to give rise to a single-arm ActRIIA homodimer Fc fusion.

Purification of various single-arm ActRIIA heterodimer Fc fusions could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to compare ligand binding selectivity of the single-arm ActRIIA heterodimer Fc fusions described above with that of an ActRIIA homodimer Fc fusion. The single-arm ActRIIA heterodimer Fc fusions and ActRIIA homodimer Fc fusions were independently captured onto the system using an anti-Fc antibody. Ligands were injected and allowed to flow over the captured receptor protein. Results are summarized in the table below, in which ligand off-rates (k_{d}) typically associated with the most effective ligand traps are denoted by gray shading.

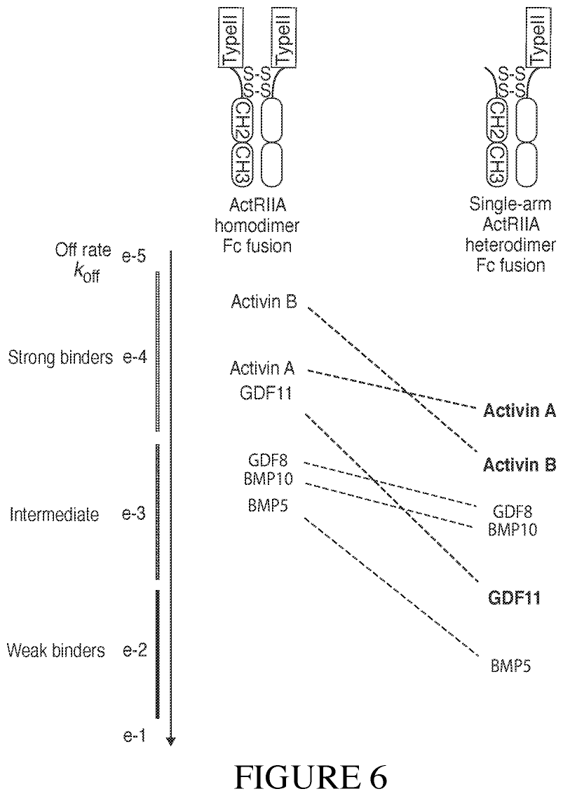

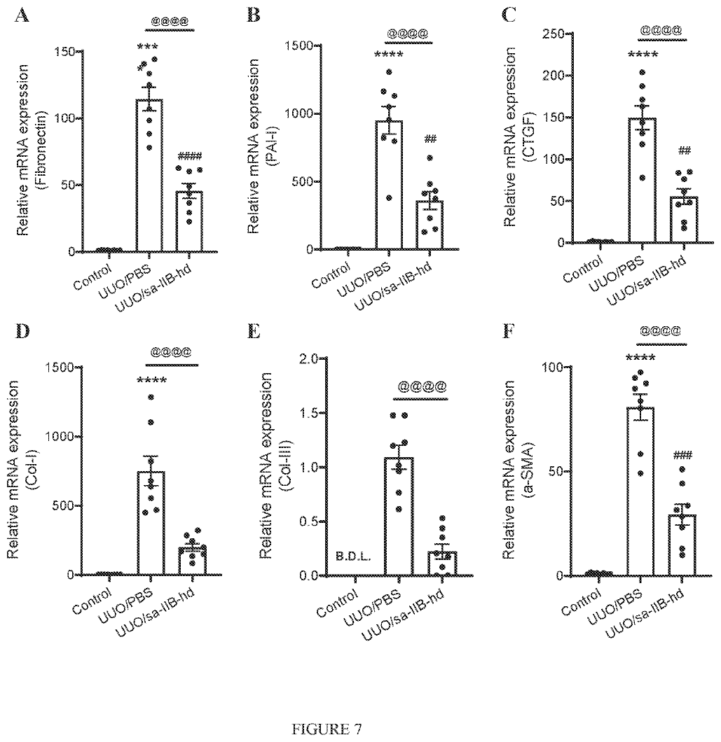

These comparative binding data indicate that a single-arm ActRIIA-Fc heterodimer Fc fusion has different ligand selectivity than an ActRIIA homodimer Fc fusion (and also different than single-armor homomeric ActRIIB-Fc—see Example 1). Whereas an ActRIIA homodimer Fc fusion exhibits preferential binding to activin B combined with strong binding to activin A and GDF11, single-arm ActRIIA heterodimer Fc fusion has a reversed preference for activin A over activin B combined with greatly enhanced selectivity for activin A over GDF11 (weak binder). See FIG. 6. In addition, single-arm ActRIIA heterodimer Fc fusion largely retains the intermediate binding to GDF8 and BMP10 observed with ActRIIA homodimer Fc fusion.

These results indicate that single-arm ActRIIA heterodimer Fc fusion is an antagonist with substantially altered ligand selectivity compared to ActRIIA homodimer Fc fusion. Accordingly, a single-arm ActRIIA heterodimer Fc fusion will be more useful than an ActRIIA homodimer Fc fusion in certain applications where such antagonism is advantageous. Examples include therapeutic applications where it is desirable to antagonize activin A preferentially over activin B while minimizing antagonism of GDF111.

Together the foregoing examples demonstrate that ActRIIA or ActRIIB polypeptides, when placed in the context of a single-arm heteromeric protein complex, form novel binding pockets that exhibit altered selectivity relative to either type of homomeric protein complex, allowing the formation of novel protein agents for possible use as therapeutic agents.

### Example 3. Single-Arm ActRIIB Heterodimer Fc Fusion Protein Treatment Suppresses Kidney Fibrosis and Inflammation and Reduces Kidney Injury

The effects of single-arm ActRIIB heterodimer Fc fusion protein on kidney disease was assessed in a mouse unilateral ureteral obstruction (UUO) model. See, e.g., Klahr and Morrissey (2002) Am J Physiol Renal Physiol 283: F861-F875.

Sixteen C57BL/6 male mice 12 weeks of age underwent left unilateral ureteral ligation twice at the level of the lower pole of kidney. After 3 days, mice were randomized into two groups: “UUO/PBS” (eight mice were injected subcutaneously with vehicle control, phosphate buffered saline (PBS), at days 3, 7, 10, and 14 after surgery) and ii) “UUO/sa-IIB-hd” (eight mice were injected subcutaneously with single-arm ActRIIB heterodimer Fc fusion protein at a dose of 10 mg/kg at days 3, 7, 10, and 14 after surgery. Both groups were sacrificed at day 17 in accordance with the relevant Animal Cam Guidelines. Half kidneys from individual animals were collected for histology analysis (H&E, and Masson's Trichrome stain), from both the UUO kidney and contralateral kidney (“Control”), and ¼ kidneys were used for RNA extraction (RNeasy Midi Kit, Qiagen, IL).

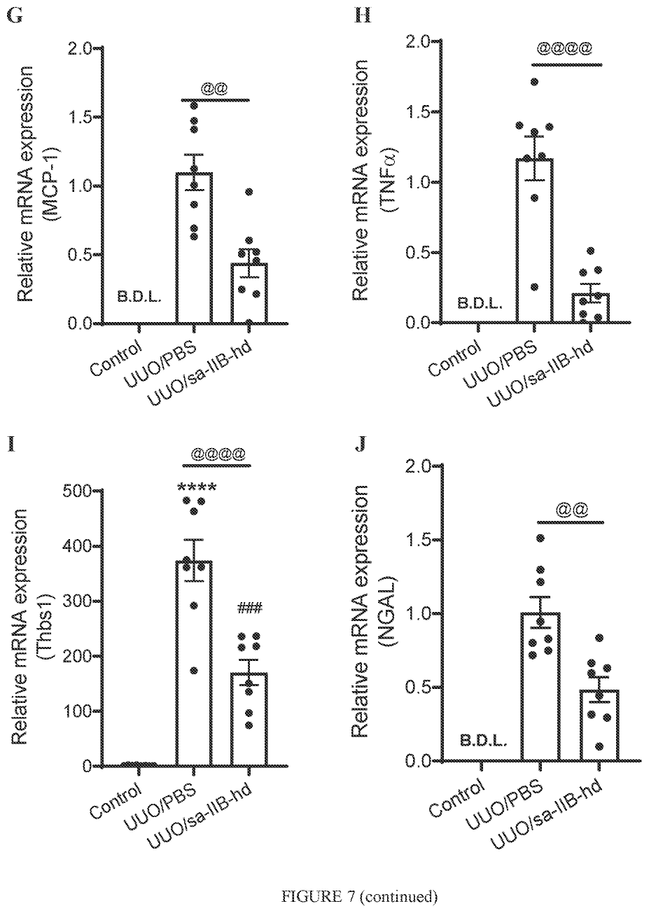

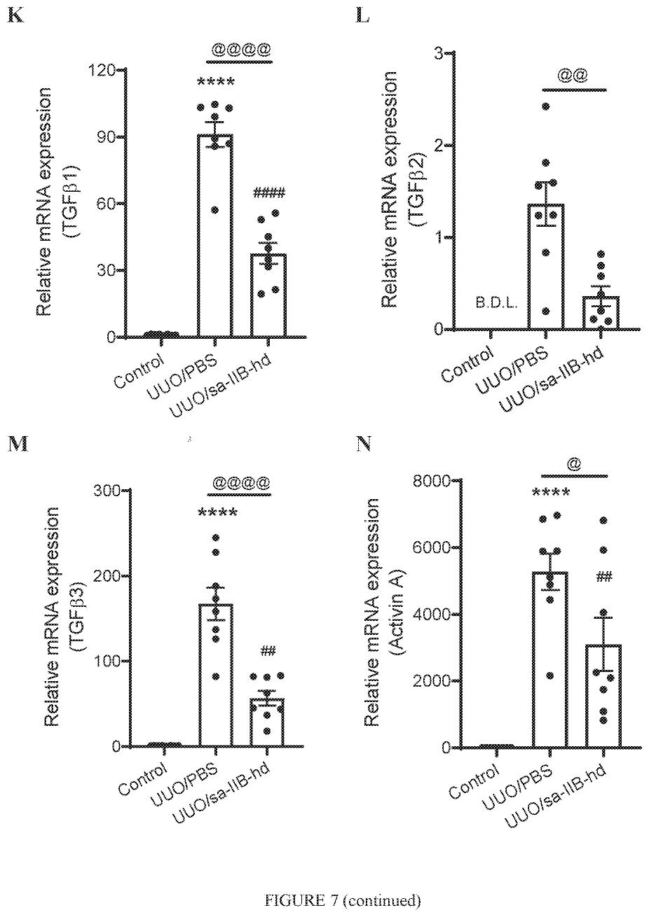

Gene expression analysis on UUO kidney samples was performed to assess levels of various genes. QRT-PCR was performed on a CFX Connect™ Real-time PCR detection system (Bio-Rad. CA) to evaluate the expression of various fibrotic genes (Fibronectin, PAI-1, CTGF, Col-I, Col-III, and a-SMA) (FIGS. 7A-7F, respectively), inflammatory genes (MCP-1 and TNFa) (FIGS. 7G-H, respectively), Thrombospondin 1 (Thbs1) (FIG. 7I), kidney injury gene (NGAL) (FIG. 7J), and TGFβ superfamily ligands (TGFβ1, TGFβ2, TGFβ3, and activin A) (FIGS. 7K-7N, respectively). Upregulation of these TGFβ superfamily ligands is highly associated with kidney fibrosis/kidney dysfunction, and they serve as a good indicator of kidney damage. In general, several fibrotic genes, including those tested here, are upregulated by TGFβ during fibrosis. Thbs1 is a direct downstream target of TGFβ, and also plays a role in regulating TGFβ activation, including during fibrosis. Measuring expression levels of Thbs1 gives an indication of the level of fibrosis, as an increase in Thbs1 expression likely means an increase in TGFβ expression. Relative to “UUO/PBS” treated mice, “UUO/sa-IIB-hd” treated mice demonstrated significantly lower expression of fibrotic and inflammatory genes, reduced upregulation of TGFβ 1/2/3, activin A, and Thbs1, and reduced kidney injury gene expression.

Together, these data demonstrate that single-arm ActRIIB heterodimer Fc fusion protein treatment suppresses kidney fibrosis and inflammation and reduces kidney injury. Moreover, these data indicate that other single-arm ActRII heterodimer Fc fusion proteins may be useful in the treatment or preventing of renal diseases or conditions including, for example, single-arm ActRIIA heterodimer Fc fusion protein.

### Example 4. Single-Arm ActRIIB Heterodimer Fc Fusion Protein Treatment Reduces Albuminuria and Improves Renal Function in Alport Mouse Model

The effects of single-arm ActRIIB heterodimer Fc fusion protein on kidney disease was assessed in a mouse Alport model (Col4a3−/−). See, e.g., Cosgrove D, et al (1996) Genes Dev 10(23): 2981-92.

Thirteen Col4a3−/− mice 4 weeks of age were randomized into two groups i) “Col4a3 Vehicle” (seven mice injected subcutaneously with vehicle control, phosphate buffered saline (PBS), twice a week) and ii) “Col4a3 sa-IIB-hd (30 mpk)” (six mice injected subcutaneously with single-arm ActRIIB heterodimer Fc fusion protein at a dose of 30 mg/kg twice a week. Urine samples were collected from six Col4a3+/+ mice (“WT”), seven Col4a3−/− mice (“Col4a3 Vehicle”), and six Col4a3−/− mice treated with single-arm ActRIIB heterodimer Fc fusion protein (“Col4a3 sa-IIB-hd (30 mpk)”) on the day before treatment starts (4 weeks) and on day 53 (7.5 weeks) to measure albumin (mouse albumin ELISA kit, Molecular Innovations, MI) and creatinine (creatinine assay kit, BioAssay Systems, CA) levels. ACR is a measurement of the ratio of albumin to creatinine in the urine. Albumin is a major protein normally present in blood, and typically little to no albumin is present in the urine when the kidneys are functioning properly. An albumin-to-creatinine ratio (ACR) is calculated to provide a more accurate indication of the how much albumin is being released into the urine. Creatinine, a byproduct of muscle metabolism, is normally released into the urine at a constant rate, allowing the creatinine measurement to serve as a way to correct for urine concentration when measuring albumin in a random urine sample. Higher ACR measurements are an indication that the kidneys are not functioning properly. Blood samples were collected from six Col4a3+/+ mice (“WT”), seven Col4a3−/− mice (“Col4a3 Vehicle”), and six Col4a3−/− mice treated with single-arm ActRIIB heterodimer Fc fusion protein (“Col4a3 sa-IIB-hd (30 mpk)”) on the day before treatment starts (4 weeks) and on day 53 (7.5 weeks) to determine blood urea nitrogen (BUN) measurements (DRI-CHEM 7000 chemistry analyzer, HESKA, CO). BUN is a measurement of the amount of nitrogen in the blood resulting from the waste product urea. Urea is typically passed out of the body through the urine. Higher levels of urea in the blood indicate that the kidneys are not functioning properly. Both groups were sacrificed at day 53 (7.5 weeks) in accordance with the relevant Animal Care Guidelines.

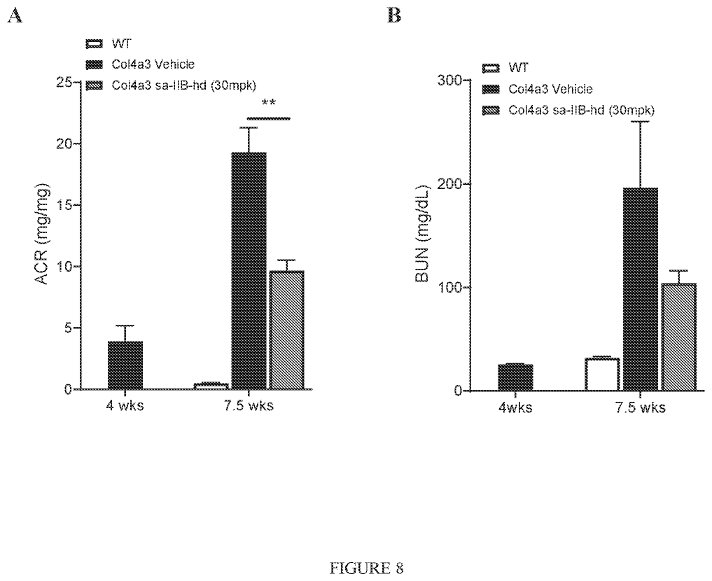

Urinary albumin to creatinine ratio (ACR) was calculated to measure albuminuria. See FIG. 8A. Albuminuria was significantly increased from 4 weeks to 7.5 weeks in Col4a3−/− mice (“Col4a3 Vehicle”) mice. Relative to Col4a3 Vehicle mice, treatment of mice with single-arm ActRIIB heterodimer Fc fusion protein (Col4a3 sa-IIB-hd mice) significantly reduced albuminuria by 49.9% (p<0.01). which was associated with decreased BUN in Col4a3 sa-IIB-hd mice (FIG. 8B).

Together, these data demonstrate that single-arm ActRIIB heterodimer Fc fusion protein treatment reduces albuminuria and improves renal function in an Alport mouse model. Moreover, these data indicate that other single-arm ActRII heterodimer Fc fusion proteins may be useful in the treatment or preventing of renal diseases or conditions (e.g., Alport's disease) including, for example, single-arm ActRIIA heterodimer Fc fusion protein.

### Example 5. Single-Arm ActRIIB Heterodimer Fc Fusion Protein Treatment Reduces Albuminuria and Prolongs Survival in the Presence of ACEi (Ramipril) in a Col4a3−/− Alport Mouse Model

The effects of single-arm ActRIIB heterodimer Fc fusion protein on kidney disease was assessed in a mouse Alport model (Col4a3−/−). See, e.g., Cosgrove D, et al (1996) Genes Dev 10(23): 2981-92.

Fifty-eight Col4a3−/− mice 6 weeks of age were treated with ramipril (ACEi, 10 mg/kg/day) in drinking water and randomized into three groups i) “Col4a3 Vehicle” (twenty-seven mice injected subcutaneously with vehicle control, phosphate buffered saline (PBS), twice a week); ii) “Col4a3 sa-IIB-hd (10 mpk)” (eleven mice injected subcutaneously with single-arm ActRIIB heterodimer Fc fusion protein at a dose of 10 mg/kg twice a week; and iii) “Col4a3 sa-IIB-hd (30 mpk)” (twenty mice injected subcutaneously with single-arm ActRIIB heterodimer Fc fusion protein at a dose of 30 mg/kg twice a week. “WT” mice are Col4a3+/+ mice with no treatment. Urine samples were collected on the day before treatment began (at 6 weeks), on day 53 (at 7.5 weeks), on day 63 (at 9 weeks), and on day 70 (at 10 weeks) to measure albumin (mouse albumin ELISA kit, Molecular Innovations, MI), neutrophil gelatinase-associated lipocalin (NGAL) (Abeam, MA), and creatinine (creatinine assay kit, BioAssay Systems, CA) levels. ACR is a measurement of the ratio of albumin to creatinine in the urine. Albumin is a major protein normally present in blood, and typically little to no albumin is present in the urine when the kidneys are functioning properly. An albumin-to-creatinine ratio (ACR) is calculated to provide a more accurate indication of the how much albumin is being released into the urine. NGAL is a marker of kidney damage and the increased levels of urinary NGAL is associated with the severity of kidney injury. Higher ACR measurements are an indication that the kidneys are not functioning properly. Treatments were continued to assess the survival for each group in accordance with the relevant Animal Care Guidelines.

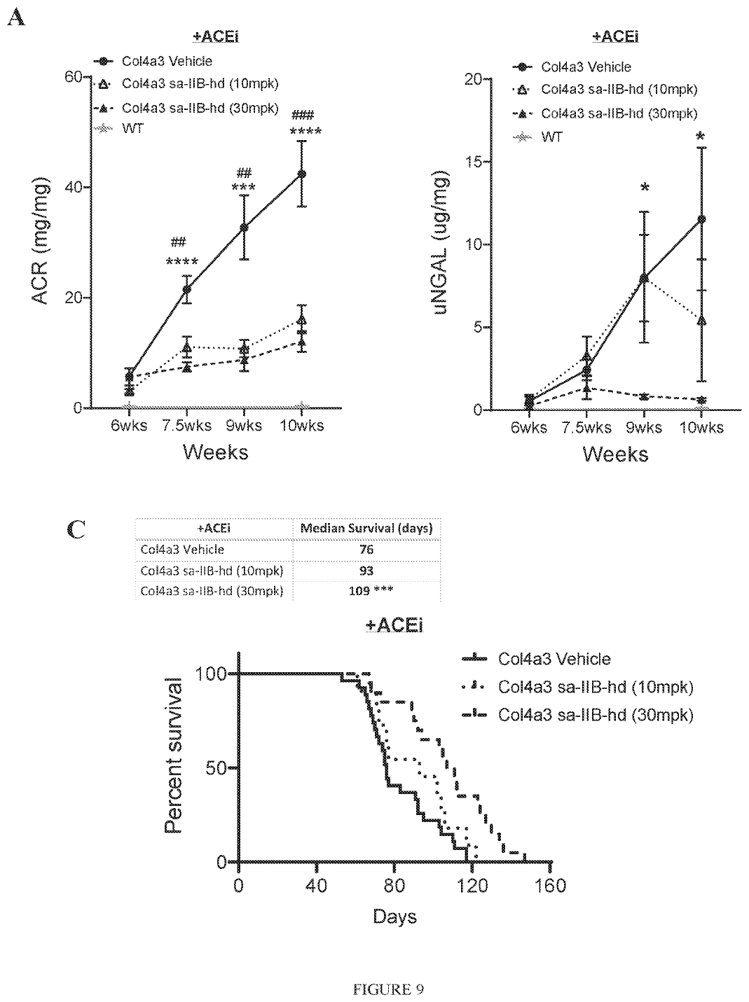

Urinary albumin to creatinine ratio (ACR) was calculated to measure albuminuria. See FIG. 9A. Regardless of ACEi treatment, albuminuria was significantly increased from 6 weeks to 10 weeks in Col4a3−/− mice (“Col4a3 Vehicle”). Relative to Col4a3 Vehicle mice, treatment of mice with single-arm ActRIIB heterodimer Fc fusion protein (Col4a3 sa-IIB-hd mice) both at 10 mg/pk and 30 mg/kg. in the presence of ACEi, significantly reduced albuminuria. In addition, 30 mg/kg treatment significantly decreased urinary NGAL (e.g., uNGAL) levels in the presence of ACEi (FIG. 9B).

As shown in FIG. 9C, in the presence of ACEi, “Col4a3 Vehicle” mice had a median survival of 76 days. Treatment of mice with single-arm ActRIIB heterodimer Fc fusion protein (Col4a3 sa-IIB-hd mice) at 10 mg/kg increased life span with a median survival of 93 days. Treatment of mice with single-arm ActRIIB heterodimer Fc fusion protein (Col4a3 sa-IIB-hd mice) at 30 mg/kg significantly increased life span with a median survival of 109 days (p<0.001).

Together, these data demonstrate that single-arm ActRIIB heterodimer Fc fusion protein treatment reduces albuminuria and prolongs survival in an Alport mouse model in the presence of an ACEi. Moreover, these data indicate that other single-arm ActRII heterodimer Fc fusion proteins may be useful in the treatment or prevention of renal diseases or conditions (e.g., Alport's disease) including, for example, single-arm ActRIIA heterodimer Fc fusion protein.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A method of treating a renal disease or condition comprising administering a single-arm ActRIIB heteromultimer to a subject in need thereof, the heteromultimer comprising a first polypeptide covalently or non-covalently associated with a second polypeptide, wherein:
a. the first polypeptide comprises an amino acid sequence of a first member of an interaction pair and an amino acid sequence of ActRIIB; and
b. the second polypeptide comprises an amino acid sequence of a second member of the interaction pair, and wherein the second polypeptide does not comprise an amino acid sequence of ActRIIB.

2. A method of treating a renal disease or condition comprising administering a single-arm ActRIIA heteromultimer to a subject in need thereof, the heteromultimer comprising a first polypeptide covalently or non-covalently associated with a second polypeptide, wherein:
a. the first polypeptide comprises an amino acid sequence of a first member of an interaction pair and an amino acid sequence of ActRIIA; and
b. the second polypeptide comprises an amino acid sequence of a second member of the interaction pair, and wherein the second polypeptide does not comprise and amino acid sequence of ActRIIA.

3. The method of claim 1, wherein the ActRIIB polypeptide comprises an amino acid sequence that is:
a. at least 70%, 80%, 85%, 90%, 91%, 92%, 93%, 94%, 94%, 95%, 96%, 97%, 98%, 99% or 100% identical to the sequence of any of SEQ ID Nos: 1, 2, 3, 4, 5, and 6; or
b. at least 70%, 80%, 85%, 90%, 91%, 92%, 93%, 94%, 94%, 95%, 96%, 97%, 98%, 99% or 100% identical to a polypeptide that begins at any one of amino acids 20, 21, 22, 23, 24, 25, 26, 27, 28, or 29 of SEQ ID NO: 1, and ends at any one of amino acids 109, 110, 111, 112, 113, 114, 115, 116, 117, 118, 119, 120, 121, 122, 123, 124, 125, 126, 127, 128, 129, 130, 131, 132, 133, or 134 of SEQ ID NO: 1.

4. The method of claim 3, wherein the ActRIIB polypeptide does not comprise an acidic amino acid at the position corresponding to L79 of SEQ ID NO: 1.

5. The method of claim 3, wherein the ActRIIB polypeptide does not comprise an aspartic acid (D) at the position corresponding to L79 of SEQ ID NO: 1.

6. The method of claim 2, wherein the ActRIIA polypeptide comprises an amino acid sequence that is:
a. at least 70%, 80%, 85%, 90%, 91%, 92%, 93%, 94%, 94%, 95%, 96%, 97%, 98%, 99% or 100% identical to the sequence of any of SEQ ID Nos: 9, 10, and 11; or
b. at least 70%, 80%, 85%, 90%, 91%, 92%, 93%, 94%, 94%, 95%, 96%, 97%, 98%, 99% or 100% identical to a polypeptide that begins at any one of amino acids 21, 22, 23, 24, 25, 26, 27, 28, 29, or 30 of SEQ ID NO: 9, and ends at any one of amino acids 110, 111, 112, 113, 114, 115, 116, 117, 118, 119, 120, 121, 122, 123, 124, 125, 126, 127, 128, 129, 130, 131, 132, 133, 134 or 135 of SEQ ID NO: 9.

7. The method of any one of claims 1-6, wherein the heteromultimer is a heterodimer.

8. The method of any of claims 1-7, wherein the first member of an interaction pair comprises a first constant region from an IgG heavy chain.

9. The method of any of claims 1-7, wherein the second member of an interaction pair comprises a second constant region from an IgG heavy chain.

10. The method of claim 8, wherein the first constant region from an IgG heavy chain is a first immunoglobulin Fc domain.

11. The method of claim 9, wherein the second constant region from an IgG heavy chain is a first immunoglobulin Fc domain.

12. The method of claim 8, wherein the first constant region from an IgG heavy chain comprises an amino acid sequence that is at least 70%, 80%, 85%, 90%, 95%, 96%, 97%, 98%, 99% or 100% identical to a sequence selected from any one of SEQ ID NOs: 14-28.

13. The method of claim 9, wherein the second constant region from an IgG heavy chain comprises an amino acid sequence that is at least 70%, 80%, 85%, 90%, 95%, 96%, 97%, 98%, 99% or 100% identical to a sequence selected from any one of SEQ ID NOs: 14-28.

14. The method of any of claims 1-13, wherein the first polypeptide comprises an amino acid sequence that is at least 70%, 80%, 85%, 90%, 95%, 96%, 97%, 98%, 99% or 100% identical to a sequence selected from any one of SEQ ID NOs: 46, 48, 55, 57, 58, 59, 60, 61, 84, 86, 88, 89, 90 and 91.

15. The method of any of claims 1-14, wherein the second polypeptide comprises an amino acid sequence that is at least 70%, 80%, 85%, 90%, 95%, 96%, 97%, 980, 99% or 100% identical to a sequence selected from any one of SEQ ID NOs: 49, 51, 62, 63, 85, and 87.

16. The method of any one of claims 1, 3-5, or 7-15, wherein the single-arm ActRIIB heteromultimer comprises a linker domain positioned between the ActRIIB polypeptide and the first member of an interaction pair.

17. The method of any one of claims 2, 6, or 7-15, wherein the single-arm ActRIIA heteromultimer comprises a linker domain positioned between the ActRIIA polypeptide and the first member of an interaction pair.

18. The method of any one of claims 16 or 17, wherein the linker domain comprises an amino acid sequence selected from any one of SEQ ID NOs: 29-44.

19. The method of any one of claims 1-18, wherein the first polypeptide and/or second polypeptide comprises one or more modified amino acid residues selected from: a glycosylated amino acid, a PEGylated amino acid, a farnesylated amino acid, an acetylated amino acid, a biotinylated amino acid, and an amino acid conjugated to a lipid moiety.

20. The method of any one of claims 1-19, wherein the first polypeptide and/or second polypeptide is glycosylated and has a glycosylation pattern obtainable from expression of the first polypeptide and/or second polypeptide in a CHO cell.

21. The method of any one of claims 1-20, wherein the heteromultimer binds to one or more ligands selected from the group consisting of activin A, activin B, GDF11, GDF8, GDF3, BMP5, BMP6, and BMP10.

22. The method of any one of claims 1, 3-5, or 7-16, 18-21, wherein the single-arm ActRIIB heteromultimer binds to activin B and GDF11.

23. The method of any one of claims 1, 3-5, or 7-16, 18-21, wherein the single-arm ActRIIB heteromultimer binds to GDF8 and activin A.

24. The method of any one of claims 2, 6, 7-15 or 17-21, wherein the single-arm ActRIIA heteromultimer binds to activin A.

25. The method of any one of claims 2, 6, 7-15, or 17-21, wherein the single-arm ActRIIA heteromultimer binds to GDF8.

26. The method of any one of claims 1-26, wherein the heteromultimer inhibits the activity of one or more ligands in a cell-based assay.

27. The method of any one of claims 1-26, wherein the renal disease or condition is Alport syndrome.

28. The method of any one of claims 1-26, wherein the renal disease or condition is focal segmental glomerulosclerosis (FSGS).

29. The method of claim 28, wherein the FSGS is primary FSGS.

30. The method of claim 28, wherein the FSGS is secondary FSGS.

31. The method of claim 28, wherein the FSGS is genetic FSGS.

32. The method of any one of claims 1-26, wherein the renal disease or condition is polycystic kidney disease.

33. The method of any one of claims 1-26, wherein the renal disease or condition is autosomal dominant polycystic kidney disease (ADPKD).

34. The method of any one of claims 1-26, wherein the renal disease or condition is autosomal recessive polycystic kidney disease (ARPKD).

35. The method of any one of claims 1-26, wherein the renal disease or condition is chronic kidney disease (CKD).

36. The method of any one of claims 1-35, wherein the subject has a decline in kidney function.

37. The method of any one of claims 1-35, wherein the method slows kidney function decline.

38. The method of any one of claims 1-37, comprising further administering to the subject an additional active agent and/or supportive therapy for treating a renal disease or condition.

39. The method of claim 38, wherein the additional active agent and/or supportive therapy for treating a renal disease or condition is selected from the group consisting of: an angiotensin receptor blocker (ARB) (e.g., losartan, irbesartan, olmesartan, candesartan, valsartan, fimasartan, azilsartan, salprisartan, and telmisartan), an angiotensin-converting enzyme (ACE) inhibitor (e.g., benazepril, captopril, enalapril, lisinopril, perindopril, ramipril, trandolapril, and zofenopril), a glucocorticoid (e.g., beclomethasone, betamethasone, budesonide, cortisone, dexamethasone, hydrocortisone, methylprednisolone, prednisolone, methylprednisone, prednisone, and triamcinolone), a calcineurin inhibitor (e.g., cyclosporine, tacrolimus), cyclophosphamide, chlorambucil, a janus kinase inhibitor (e.g., tofacitinib), an mTOR inhibitor (e.g., sirolimus, everolimus), an IMDH inhibitor (e.g., azathioprine, leflunomide, mycophenolate), a biologic (e.g., abatacept, adalimumab, anakinra, basiliximab, certolizumab, daclizumab, etanercept, fresolimumab, golimumab, infliximab, ixekizumab, natalizumab, rituximab, secukinumab, tocilizumab, ustekinumab, vedolizumab), a statin (e.g., benazepril, valsartan, fluvastatin, pravastatin), lademirsen (anti-miRNA-21), bardoxolone methyl, Achtar gel, tolvaptan, abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-11, galactose, GC1008, GFB-887, isotretinoin, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (K_{D}019), tetracosactin, tripterygium wilfordii (TW), valproic acid, VAR-200, venglustat (GZ402671), verinurad, voclosporin, VX-147, kidney dialysis, kidney transplant, mescnchymal stem cell therapy, bone marrow stem cells, lipoprotein removal, a Liposorber LA-15 device, plasmapheresis, plasma exchange, and a change in diet (e.g., dietary sodium intake).

40. The method of claim 38, wherein the additional active agent and/or supportive therapy for treating a renal disease or condition is an angiotensin receptor blocker (ARB) selected from the group consisting of losartan, irbesartan, olmesartan, candesartan, valsartan, fimasartan, azilsartan, salprisartan, and telmisartan.

41. The method of claim 38, wherein the additional active agent and/or supportive therapy for treating a renal disease or condition is an angiotensin-converting enzyme (ACE) inhibitor selected from the group consisting of benazepril, captopril, enalapril, lisinopril, perindopril, ramipril, trandolapril, and zofenopril.

42. The method of claim 38, wherein the additional active agent and/or supportive therapy for treating a renal disease or condition is a combination of an ARB and an ACE inhibitor.

43. The method of any one of claims 1-42, wherein the subject has proteinuria.

44. The method of any one of claims 1-42, wherein the subject has albuminuria.

45. The method of any one of claims 1-42, wherein the subject has moderate albuminuria.

46. The method of any one of claims 1-42, wherein the subject has severe albuminuria.

47. The method of any one of claims 1-46, wherein the method reduces severity, occurrence and/or duration of one or more of albuminuria, proteinuria, microalbuminuria, and macroalbuminuria in a subject in need thereof.

48. The method of claim 45, wherein the subject has an albumin-creatinine ratio (ACR) of between about 30 and about 300 mg albumin per 24 hours of urine collection.

49. The method of claim 45, wherein the subject has an ACR of between about 30 and about 300 mg albumin/g of creatinine.

50. The method of claim 46, wherein the subject has an albumin-creatinine ratio (ACR) of above about 300 mg albumin/24 hours.

51. The method of claim 46, wherein the subject has an ACR of above about 300 mg albumin/g of creatinine.

52. The method of any one of claims 1-44, wherein the subject has Stage Δ1 albuminuria.

53. The method of any one of claims 1-44, wherein the subject has Stage Δ2 albuminuria.

54. The method of any one of claims 1-44, wherein the subject has Stage Δ3 albuminuria.

55. The method of any one of claims 1-44, wherein the method reduces severity, occurrence and/or duration of Stage A 1 albuminuria.

56. The method of any one of claims 1-44, wherein the method reduces severity, occurrence and/or duration of Stage Δ2 albuminuria.

57. The method of any one of claims 1-44, wherein the method reduces severity, occurrence and/or duration of Stage A 3 albuminuria.

58. The method of any one of claims 1-44, wherein the method delays or prevents a subject with Stage Δ1 albuminuria from progressing to Stage Δ2 albuminuria.

59. The method of any one of claims 1-44, wherein the method delays or prevents a subject with Stage Δ2 from progressing to Stage Δ3 albuminuria.

60. The method of any one of claims 1-44, wherein the method delays and/or prevents worsening of albuminuria stage progression in a subject in need thereof.

61. The method of any one of claims 1-44, wherein the method improves albuminuria classification in a subject by one or more stages.

62. The method of any one of claims 1-61, wherein the method reduces an ACR of the subject.

63. The method of claim 62, wherein the method reduces the subject's ACR by between about 0.1 and about 100.0 mg albumin/g creatinine (e.g., by between about 0.1 and about 2.5 mg albumin/g, between about 2.5 and about 3.5 mg albumin/g creatinine, between about 3.5 and about 5.0 mg albumin/g creatinine, between about 5.0 and about 7.5 mg albumin/g creatinine, between about 7.5 and about 10.0 mg albumin/g creatinine, between about 10.0 and about 15.0 mg albumin/g creatinine, between about 15.0 and about 20.0 mg albumin/g creatinine, between about 20.0 and about 25.0 mg albumin/g creatinine, between about 30.0 and about 35.0 mg albumin/g creatinine, between about 40.0 and about 45.0 mg albumin/g creatinine, between about 45.0 and about 50.0 mg albumin/g creatinine, between about 50.0 and about 60.0 mg albumin/g creatinine, between about 60.0 and about 70.0 mg albumin/g creatinine, between about 70.0 and about 80.0 mg albumin/g creatinine, between about 80.0 and about 90.0 mg albumin/g creatinine, between about 90.0 and about 100.0 mg albumin/g creatinine).

64. The method of claim 62, wherein the method reduces the subject's ACR by at least 2.5% (e.g., 5%, 10%, 15%, 20%, 25%, 30%, 40%, 50%, 60%, 70%, 80%, 90%, 95%, or 99%) compared to a baseline measurement.

65. The method of any one of claims 1-64, wherein the method reduces a urinary protein-creatinine ratio (UPCR) of the subject.

66. The method claim 65, wherein the method reduces the subject's UPCR by between about 0.1 and about 100.0 mg urinary protein/mg creatinine (e.g., by between about 0.1 and about 2.5 mg urinary protein/mg creatinine, between about 2.5 and about 3.5 mg urinary protein/mg creatinine, between about 3.5 and about 5.0 mg urinary protein/mg creatinine, between about 5.0 and about 7.5 mg urinary protein/mg creatinine, between about 7.5 and about 10.0 mg urinary protein/mg creatinine, between about 10.0 and about 15.0 mg urinary protein/mg creatinine, between about 15.0 and about 20.0 mg urinary protein/mg creatinine, between about 20.0 and about 25.0 mg urinary protein/mg creatinine, between about 30.0 and about 35.0 mg urinary protein/mg creatinine, between about 40.0 and about 45.0 mg urinary protein/mg creatinine, between about 45.0 and about 50.0 mg urinary protein/mg creatinine, between about 50.0 and about 60.0 mg urinary protein/mg creatinine, between about 60.0 and about 70.0 mg urinary protein/mg creatinine, between about 70.0 and about 80.0 mg urinary protein/mg creatinine, between about 80.0 and about 90.0 mg urinary protein/mg creatinine, between about 90.0 and about 100.0 mg urinary protein/mg creatinine).

67. The method claim 65, wherein the method reduces the subject's UPCR by between about 0.1 and about 100.0 g urinary protein/g creatinine (e.g., by between about 0.1 and about 2.5 g urinary protein/g creatinine, between about 2.5 and about 3.5 g urinary protein/g creatinine, between about 3.5 and about 5.0 g urinary protein/g creatinine, between about 5.0 and about 7.5 g urinary protein/g creatinine, between about 7.5 and about 10.0 g urinary protein/g creatinine, between about 10.0 and about 15.0 g urinary protein/g creatinine, between about 15.0 and about 20.0 g urinary protein/g creatinine, between about 20.0 and about 25.0 g urinary protein/g creatinine, between about 30.0 and about 35.0 g urinary protein/g creatinine, between about 40.0 and about 45.0 g urinary protein/g creatinine, between about 45.0 and about 50.0 g urinary protein/g creatinine, between about 50.0 and about 60.0 g urinary protein/g creatinine, between about 60.0 and about 70.0 g urinary protein/g creatinine, between about 70.0 and about 80.0 g urinary protein/g creatinine, between about 80.0 and about 90.0 g urinary protein/g creatinine, between about 90.0 and about 100.0 g urinary protein/g creatinine).

68. The method of claim 65, wherein the method reduces the subject's absolute UPCR by greater than or equal to 0.5 g urinary protein/g creatinine compared to a baseline measurement.

69. The method of claim 65, wherein the method reduces the subject's UPCR to less than 0.5 g urinary protein/g creatinine compared to a baseline measurement.

70. The method of claim 65, wherein the method reduces the subject's UPCR to less than 0.3 g urinary protein/g creatinine compared to a baseline measurement.

71. The method of claim 65, wherein the method reduces the subject's UPCR by at least 2.5% (e.g., 5%, 10%, 15%, 20%, 25%, 30%, 40%, 50%, 60%, 70%, 80%, 90%, 95%, or 99%) compared to a baseline measurement.

72. The method of claim 65, wherein the method reduces the subject's UPCR by greater than or equal to 30% compared to a baseline measurement.

73. The method of claim 65, wherein the method reduces the subject's UPCR by greater than or equal to 40% compared to a baseline measurement.

74. The method of claim 65, wherein the method reduces the subject's UPCR by greater than or equal to 50% compared to a baseline measurement.

75. The method of any one of claims 1-74, wherein the method increases the subject's estimated glomerular filtration rate (eGFR) and/or glomerular filtration rate (GFR).

76. The method of claim 75, wherein the eGFR is measured using serum creatinine, age, ethnicity, and gender variables.

77. The method of claim 75, wherein the eGFR is measured using one or more of Cockcroft-Gault formula, Modification of Diet in Renal Disease (MDRD) formula, CKD-EPI formula, Mayo quadratic formula, and Schwartz formula.

78. The method of claim 75, wherein the eGFR and/or GFR is increased by at least 2.5% (e.g., 5%, 10%, 15%, 20%, 25%, 30%, 40%, 50%, 60%, 70%, 80%, 90%, 95%, or 99%) compared to a baseline measurement.

79. The method of claim 75, wherein the eGFR and/or GFR is increased by greater than or equal to 30% compared to a baseline measurement.

80. The method of claim 75, wherein the eGFR and/or GFR is increased by greater than or equal to 40% compared to a baseline measurement.

81. The method of claim 75, wherein the eGFR and/or GFR is increased by about 1 mL/min/1.73 m² (e.g., 3, 5, 7, 9, 10, 15, 20, 25, 30, 35, 40, 45, 50, 55, 60, 65, 70, 75, 80, 85, 90, 95, or 100 mL/min/1.73 m²) compared to a baseline measurement.

82. The method of claim 75, wherein the eGFR and/or GFR is increased by about 1 mL/min/year (e.g., 2, 3, 5, 7, 9, 10, 15, 20, 25, 30, 35, 40, 45, 50, 55, 60, 65, 70, 75, 80, 85, 90, 95, or 100 mL/min/year) compared to a baseline measurement.

83. The method of claim 75, wherein the eGFR and/or GFR is increased by greater than or equal to 1 mL/min/year compared to a baseline measurement.

84. The method of claim 75, wherein the eGFR and/or GFR is increased by greater than or equal to 3 mL/min/year compared to a baseline measurement.

85. The method of any one of claims 1-84, wherein the renal disease or condition is evaluated in stages of chronic kidney disease (CKD).

86. The method of any one of claims 1-84, wherein the subject has stage one chronic kidney disease (CKD).

87. The method of any one of claims 1-84, wherein the subject has stage two chronic kidney disease (CKD).

88. The method of any one of claims 1-84, wherein the subject has stage three chronic kidney disease (CKD).

89. The method of any one of claims 1-84, wherein the subject has stage four chronic kidney disease (CKD).

90. The method of any one of claims 1-84, wherein the subject has stage five chronic kidney disease (CKD).

91. The method of any one of claims 1-84, wherein the method reduces severity, occurrence and/or duration of Stage 1 CKD.

92. The method of any one of claims 1-84, wherein the method reduces severity, occurrence and/or duration of Stage 2 CKD.

93. The method of any one of claims 1-84, wherein the method reduces severity, occurrence and/or duration of Stage 3 CKD.

94. The method of any one of claims 1-84, wherein the method reduces severity, occurrence and/or duration of Stage 3a CKD.

95. The method of any one of claims 1-84, wherein the method reduces severity, occurrence and/or duration of Stage 3b CKD.

96. The method of any one of claims 1-84, wherein the method reduces severity, occurrence and/or duration of Stage 4 CKD.

97. The method of any one of claims 1-84, wherein the method reduces severity, occurrence and/or duration of Stage 5 CKD.

98. The method of any one of claims 1-84, wherein the method prevents or delays a subject with Stage 1 CKD from progressing to Stage 2 CKD.

99. The method of any one of claims 1-84, wherein the method prevents or delays a subject with Stage 2 CKD from progressing to Stage 3 CKD.

100. The method of any one of claims 1-84, wherein the method prevents or delays a subject with Stage 2 CKD from progressing to Stage 3a CKD.

101. The method of any one of claims 1-84, wherein the method prevents or delays a subject with Stage 3a CKD from progressing to Stage 3b CKD.

102. The method of any one of claims 1-84, wherein the method prevents or delays a subject with Stage 3 CKD from progressing to Stage 4 CKD.

103. The method of any one of claims 1-84, wherein the method prevents or delays a subject with Stage 3b CKD rom progressing to Stage 4 CKD.

104. The method of any one of claims 1-84, wherein the method prevents or delays a subject with Stage 4 CKD rom progressing to Stage 5 CKD.

105. The method of any one of claims 1-104, wherein the method delays and/or prevents worsening of CKD stage progression in a subject in need thereof.

106. The method of any one of claims 1-105, wherein the method improves renal damage CKD classification in a subject by one or more stages.

107. The method of any one of claims 1-106, wherein the method reduces total kidney volume in a subject.

108. The method of claim 107, wherein the total kidney volume is reduced by at least 2.5% (e.g., 5%, 10%, 15%, 20%, 25%, 30%, 40%, 50%, 60%, 70%, 80%, 90%, 95%, or 99%) compared to a baseline measurement.

109. The method of any one of claims 1-108, wherein the method reduces the subject's blood urea nitrogen (BUN).

110. The method of claim 109, wherein the BUN is reduced by at least 2.5% (e.g., 5%, 10%, 15%, 20%, 25%, 30%, 40%, 50%, 60%, 70%, 80%, 90%, 95%, or 99%) compared to a baseline measurement.

111. The method of any one of claims 1-110, wherein the method reduces the subject's urine Neutrophil Gelatinase-Associated Lipocalin (uNGAL) concentration.

112. The method of claim 111, wherein the uNGAL is reduced by at least 2.5% (e.g., 5%, 10%, 15%, 20%, 25%, 30%, 40%, 50%, 60%, 70%, 80%, 90%, 95%, or 99%) compared to a baseline measurement.

113. The method of any one of claims 1-112, wherein the subject has a uNGAL measurement of <50 ng/mL, is an indication of low risk of acute kidney injury.

114. The method of any one of claims 1-112, wherein the subject has a uNGAL measurement of between about 50 and about 149 ng/mL, an indication of equivocal risk of acute kidney injury.

115. The method of any one of claims 1-112, wherein the subject has a uNGAL measurement of between about 150 and about 300 ng/mL, an indication of moderate risk of acute kidney injury.

116. The method of any one of claims 1-112, wherein the subject has a uNGAL measurement of >300 ng/mL, an indication of high risk of acute kidney injury.

117. The method of any one of claims 1-116, wherein the method reduces the subject's uNGAL by between about 0.1 and about 300.0 ng/mL (e.g., by between about 0.1 and about 50 ng/mL, by between about 0.1 and about 100.0 ng/mL, by between about 0.1 and about 150.0 ng/mL, by between about 0.1 and about 200.0 ng/mL, by between about 0.1 and about 250.0 ng/mL, by between about 0.1 and about 300.0 ng/mL, by between about 0.1 and about ng/mL, by between about 25 and about 50 ng/mL, by between about 50 and about 100 ng/mL, by between about 100 and about 150 ng/mL, by between about 150 and about 200 ng/mL, by between about 200 and about 250 ng/mL, by between about 250 and about 300 ng/mL, by more than 300 ng/mL).

118. The method of any one of claims 1-117, wherein the method prevents or delays clinical worsening of a renal disease or condition.

119. The method of any one of claims 1-118, wherein the method reduces risk of hospitalization for one or more complications associated with a renal disease or condition.

120. The method of any one of claims 1-119, wherein the single-arm ActRIIB heteromultimer or single-arm ActRIIA heteromultimer is administered subcutaneously.

121. The method of any one of claims 1-120, wherein the single-arm ActRIIB heteromultimer or single-arm ActRIIA heteromultimer is administered once every two weeks.

122. The method of any one of claims 1-120, wherein the single-arm ActRIIB heteromultimer or single-arm ActRIIA heteromultimer is administered once every three weeks.

123. The method of any one of claims 1-120, wherein the single-arm ActRIIB heteromultimer or single-arm ActRIIA heteromultimer is administered once every four weeks.
